(19) United States
(12) Patent Application Publication
(10) Pub. No.: US 2025/0302928 A1
(43) Pub. Date: Oct. 2, 2025

(54) TREATMENT OF CERVICAL DYSTONIA

(71) Applicant: IPSEN BIOPHARM LIMITED, Wrexham (GB)

(72) Inventors: Nicolae Grigore, Wrexham (GB); Laurent Pons, Wrexham (GB)

(21) Appl. No.: 18/851,566

(22) PCT Filed: Mar. 23, 2023
(86) PCT No.: PCT/GB2023/050742

§ 371 Date: Sep. 26, 2024

(30) Foreign Application Priority Data
Apr. 29, 2022 (GB) 2206353.1

(51) Int. Cl. A61K 38/48; A61P 21/00; A61P 25/14

(52) U.S. Cl. CPC A61K 38/4893; A61P 21/00; A61P 25/14; C12Y 304/24069

(57) ABSTRACT

The present invention is directed inter alia to a modified botulinum neurotoxin A (BoNT/A) for use in treating cervical dystonia, wherein the modified BoNT/A is administered by intramuscular injection to an affected neck muscle of a subject, wherein the modified BoNT/A is administered by way of a unit dose of greater than 17,000 pg of modified BoNT/A, wherein at least a single unit dose is administered to the affected neck muscle, wherein the total dose administered during the treatment is up to 400,000 pg of modified BoNT/A, and wherein the modified BoNT/A comprises a BoNT/A light-chain and translocation domain, and a BoNT/B receptor binding domain (HC domain). Also provided are corresponding methods, uses, unit dosage forms, and kits.

### FIELD OF THE INVENTION

The present invention relates to treatment of cervical dystonia.

### BACKGROUND

Cervical dystonia (also known as spasmodic torticollis) is a chronic neurological movement disorder typically associated with extreme pain. The disorder causes the neck of an affected subject to involuntarily turn to the left, right, upwards, and/or downwards. Both agonist and antagonist muscles may contract simultaneously during dystonic movement.

The disorder typically presents with relatively mild symptoms, such as an invisible tremor of the head for a few months at onset. Other early/progressive symptoms may include the head turning, pulling, and/or tilting in sudden movements. Yet further early/progressive symptoms typically include sustained/prolonged involuntary head positioning. Involuntary neck muscle spasms tend to increase in frequency and strength over time prior to reaching a plateau. Subjects with cervical dystonia may also experience muscle hypertrophy, neck pain, dysarthria, and/or tremor.

The symptoms of cervical dystonia may involve any neck muscles of a subject and the head posture can vary. Typically the most common abnormal posture associated with cervical dystonia is the twisting of the chin toward a shoulder so that the head rotates sideways (torticollis). Other abnormal postures associated with cervical dystonia may include anterocollis, where the head is tipped forward, retrocollis, where the head is tilted backwards, or laterocollis, where the head is tilted toward one side. There can also be shifting of the head on the shoulders in an anterior sagittal shift (a forward shift) or posterior sagittal shift (a backwards shift). However, most commonly, cervical dystonia presents with complex symptoms in which a subject exhibits several angles of head movement.

Current treatment options include oral medications (e.g. dopamine blocking agents), deep brain stimulation, botulinum neurotoxins, and selective surgical denervation of nerves triggering muscle contractions. Conventional oral medications are associated with a number of severe side-effects, while deep brain stimulation and surgical denervation are invasive, have associated risks of complications, and/or can be ineffective.

An example of a conventional botulinum neurotoxin serotype A (BoNT/A) treatment for cervical dystonia is Dysport®, which is a medicinal product containing drug substance BoNT/A haemagglutinin complex (BTX-A-HAC) isolated and purified from Clostridium botulinum type A strain. Several other medicinal BoNT/A products naturally produced by Clostridium botulinum are also on the market (e.g. BOTOX® and XEOMIN®).

By paralysing a dystonic antagonist muscle, BoNT/A may allow the agonist muscle to move freely. In more detail, BoNT/A selectively inhibits the release of acetylcholine from the presynaptic nerve terminals and thus blocks cholinergic transmission at the neuromuscular junction inducing a reduction in the muscle contraction and muscle tone, causing the injected muscles to relax. However, the duration of action of the currently available BoNT/A products is about 12 to 14 weeks, which is when the new nerve endings sprout allowing the nerve function to return to normal, and the original symptoms reappear. Consequently, for the effect to be maintained, injections need to be repeated periodically. Thus, the frequency of BoNT/A injections is an important consideration for the treatment of cervical dystonia, considering the chronicity of the condition and long-term nature of the treatment required. Indeed, it has an impact on the direct and indirect health costs involved for the patients and caregivers, the logistics for injections within the hospitals/clinics, and, most importantly, the quality of life of patients.

Dysport® is approved for the treatment of cervical dystonia with a maximum total dose per treatment session of 1,000 Units (see FIG. 1). A clinician is required to administer Dysport® to neck muscles of the subject up to the upper threshold of 1,000 Units total per treatment session. The clinician is forced to make difficult choices during treatment of a patient. In other words, in conventional treatment regimens, a clinician must find a balance between the relatively low total amount of BoNT/A that can be administered (1,000 Units—necessitated by the highly toxic nature of BoNT/A) and the effective amount at a plurality of different muscles. Hence, certain muscles are neglected while others receive a suboptimal amount of BoNT/A, resulting in suboptimal therapy.

Moreover, the conventional cervical dystonia treatment regimens are complicated and result in clinicians under-dosing in an effort to avoid toxicity to the patient. There is thus a need for a convenient, safe, and effective single dose unit and a corresponding guide to the number of units that can be administered to an affected neck muscle (e.g. including the number of injection sites per muscle) in a treatment session without resultant patient toxicity.

In conclusion, there is a need for an improved treatment for cervical dystonia that would allow an individualised patient-centric approach to tailor the treatment according to the targeted clinical pattern permitting different combinations of affected neck muscles to be injected depending on the distribution, extent and severity of the cervical dystonia, while avoiding toxicity and providing a longer-lasting treatment (resulting in less frequent administration).

The present invention overcomes one or more of the above-mentioned problems.

### SUMMARY OF THE INVENTION

The present inventors have surprisingly found that a modified BoNT/A finds particular utility in treating cervical dystonia. The modified BoNT/A of the invention comprises a BoNT/A light-chain and translocation domain (H_{N} domain) and a BoNT/B receptor binding domain (H_{C} domain), which may result in a modified BoNT/A that exhibits increased retention at (reduced diffusion away from) a site of administration and/or increased duration of action (e.g. 6-9 months).

Advantageously, modified BoNT/A has a safety profile that is improved when compared to unmodified BoNT/A (e.g. Dysport®). This improved safety profile may be expressed by the high Safety Ratio described herein for the modified BoNT/A.

Based on the pre-clinical and clinical data herein (see Examples) it has been shown that a higher total amount of modified BoNT/A can be administered to a subject while achieving a similar safety profile to unmodified BoNT/A (e.g. Dysport®) while at such high doses. Advantageously, clinical data herein (see Example 9) has indicated that higher unit doses and total amounts of modified BoNT/A can be administered to a subject while achieving a similar safety profile to unmodified BoNT/A (e.g. Dysport®) while at such high doses. Thus, more modified BoNT/A can be injected and/or can be injected at a greater number of neck muscles/sites in the treatment of cervical dystonia before reaching the maximum total dose. This is a significant and advantageous finding, and yields an improved treatment of cervical dystonia while providing clinicians with a greater range of treatment options. The treatment may be improved in that it provides for longer-lasting treatment (resulting in less frequent administration) and/or is capable of being tailored for the subject and/or results in an improved quality of life of a subject when compared to treatment with unmodified BoNT/A (e.g. Dysport®). Hence, the treatment of the invention is improved compared to conventional treatment regimens.

Moreover, the present invention provides a convenient, safe, and effective single unit dose as well as a total (maximum) dosage that can be safely administered in a single treatment. The present invention also provides a corresponding guide to the number of times at which said unit dose can be administered to a neck muscle (e.g. including the number of injection sites per muscle) without resultant patient toxicity. Treatment of cervical dystonia in accordance with the present invention is thus much less complicated for the clinician and helps avoid under-dosing and/or over-dosing. Furthermore, treatment according to the invention is much more satisfactory to the patient, as it is better tailored to the patient's needs, when compared to conventional cervical dystonia treatments.

### DETAILED DESCRIPTION

In one aspect, the invention provides a modified botulinum neurotoxin A (BoNT/A) for use in treating cervical dystonia, wherein the modified BoNT/A is administered by intramuscular injection to an affected neck muscle of a subject,
wherein the modified BoNT/A is administered by way of a unit dose of greater than 17,000 pg of modified BoNT/A,
wherein at least a single unit dose is administered to the affected neck muscle,
wherein the total dose administered during the treatment is up to 400,000 pg of modified BoNT/A, and
wherein the modified BoNT/A comprises a BoNT/A light-chain and translocation domain, and a BoNT/B receptor binding domain (H_{C} domain).

In a related aspect, the invention provides a modified BoNT/A for use in treating cervical dystonia of a subject for a longer duration than that treated by an unmodified BoNT/A (e.g. SEQ ID NO: 2 [such as a di-chain form of SEQ ID NO: 2]), wherein the modified BoNT/A is administered by intramuscular injection to an affected neck muscle of the subject,
wherein the modified BoNT/A is administered by way of a unit dose of greater than 17,000 pg of modified BoNT/A,
wherein at least a single unit dose is administered to the affected neck muscle,
wherein the total dose administered during the treatment is up to 400,000 pg of modified BoNT/A,
wherein the modified BoNT/A comprises a BoNT/A light-chain and translocation domain, and a BoNT/B receptor binding domain (H_{C} domain).

The term “treating cervical dystonia of a subject for a longer duration than that treated by an unmodified BoNT/A” may mean that one or more symptoms of cervical dystonia of the subject are reduced for a longer time period (e.g. 6-9 months) following administration of a modified BONT/A of the invention, when compared to administration of an unmodified BoNT/A. Said duration of action may be at least 1.25×, 1.5×, 1.75×, 2.0×, or 2.25× greater. The duration of action of modified BoNT/A may be between 6 and 9 months. For example, a duration of action may be at least: 4.5 months (from onset), 5.0 months, 5.5 months, 6 months, 6.5 months, 7.0 months, 7.5 months, 8.0 months, 8.5 months or 9.0 months. In particular embodiments, a duration of action may be greater than 9.0 months. Said reduction may be determined by comparison to an equivalent control subject exhibiting equivalent symptoms that has been treated with an unmodified BoNT/A. At a time period where the severity of one or more symptoms of the control subject are substantially the same (e.g. the same) as before unmodified BoNT/A treatment, a subject treated with a modified BoNT/A according to the invention may exhibit an improvement in the equivalent one or more symptoms of at least 5%, 10%, 25%, or 50% when compared to the severity of the one or more symptoms before treatment with the modified BoNT/A. The unmodified BoNT/A is preferably SEQ ID NO: 2 present in a di-chain form.

In one aspect, the invention provides a method for treating cervical dystonia, the method comprising administering a modified BoNT/A by intramuscular injection to an affected neck muscle of a subject,
wherein the modified BoNT/A is administered by way of a unit dose of greater than 17,000 pg of modified BoNT/A,
wherein at least a single unit dose is administered to the affected neck muscle,
wherein the total dose administered during the treatment is up to 400,000 pg of modified BoNT/A, and
wherein the modified BoNT/A comprises a BoNT/A light-chain and translocation domain, and a BoNT/B receptor binding domain (H_{C} domain).

In a related aspect, the invention provides a method for treating cervical dystonia of a subject for a longer duration than that treated by an unmodified BoNT/A (e.g. SEQ ID NO: 2 [such as a di-chain form of SEQ ID NO: 2]), the method comprising administering a modified BoNT/A by intramuscular injection to an affected neck muscle of the subject,
wherein the modified BoNT/A is administered by way of a unit dose of greater than 17,000 pg of modified BoNT/A,
wherein at least a single unit dose is administered to the affected neck muscle,
wherein the total dose administered during the treatment is up to 400,000 pg of modified BoNT/A,
wherein the modified BoNT/A comprises a BoNT/A light-chain and translocation domain, and a BoNT/B receptor binding domain (H_{C} domain).

In one aspect, the invention provides the use of a modified botulinum neurotoxin A (BoNT/A) in the manufacture of a medicament for treating cervical dystonia, wherein the modified BONT/A is administered by intramuscular injection to an affected neck muscle of a subject,
wherein the modified BoNT/A is administered by way of a unit dose of greater than 17,000 pg of modified BoNT/A,
wherein at least a single unit dose is administered to the affected neck muscle,
wherein the total dose administered during the treatment is up to 400,000 pg of modified BoNT/A, and
wherein the modified BoNT/A comprises a BoNT/A light-chain and translocation domain, and a BoNT/B receptor binding domain (H_{C} domain).

In a related aspect, the invention provides use of a modified BoNT/A in the manufacture of a medicament for treating cervical dystonia of a subject for a longer duration than that treated by an unmodified BoNT/A (e.g. SEQ ID NO: 2 [such as a di-chain form of SEQ ID NO: 2]), wherein the modified BoNT/A is administered by intramuscular injection to an affected neck muscle of the subject,
wherein the modified BoNT/A is administered by way of a unit dose of greater than 17,000 pg of modified BoNT/A,
wherein at least a single unit dose is administered to the affected neck muscle,
wherein the total dose administered during the treatment is up to 400,000 pg of modified BoNT/A,
wherein the modified BoNT/A comprises a BoNT/A light-chain and translocation domain, and a BoNT/B receptor binding domain (H_{C} domain).

The unit dose may be greater than 17,000 pg of modified BoNT/A. An upper limit of the unit dose range may be 40,000, 39,000, 38,000, 37,000, 36,000, 35,000, 30,000, 25,000, 24,000, 22,000, 20,000, or 18,000, pg of modified BoNT/A, preferably the upper limit is 38,000 pg. A lower limit of the unit dose range may be 17,500, 18,000, 20,000, 22,000, 24,000, 25,000, 26,000, 27,000, 28,000, 29,000, 30,000, 35,000, 36,000, 37,000, 38,000 or 39,000 pg of modified BoNT/A, preferably the lower limit is 17,500 pg or 25,000 pg. Preferably, the unit dose of modified BoNT/A is greater than 17,000 pg up to 40,000 pg of modified BoNT/A, e.g. greater than 17,000 pg up to 36,000 pg, or 20,000 pg to 39,000 pg. Most preferably a unit dose of modified BoNT/A is 22,000 to 38,000 pg, such as 24,000 to 36,000 pg or 25,000 to 36,000 pg. The unit dose may be 25,000 pg up to 40,000 pg of modified BoNT/A. In preferred embodiments, a unit dose of modified BoNT/A is 24,000, 25,000, 30,000 or 36,000 pg, e.g. 25,000 or 36,000 pg. In more preferred embodiments, a unit dose of modified BoNT/A is 30,000 or 36,000 pg (e.g. 36,000 pg).

The unit dose may be 20,000 pg to 30,000 pg, such as 24,000 pg to 26,000 pg of modified BONT/A. Most preferably, the unit dose is 25,000 pg of modified BoNT/A.

The unit dose may be 30,000 pg to 40,000 pg, such as 35,000 pg to 37,000 pg of modified BONT/A. Most preferably, the unit dose is 36,000 pg of modified BoNT/A.

A total dose administered when carrying out the treatment regimen of the present invention may be up to 400,000 pg. In other words, the total amount of modified BoNT/A administered at a given treatment session may be up to 400,000 pg. The total dose may be up to 380,000, 360,000, 340,000, 320,000, 300,000, 280,000, 260,000, 250,000, 240,000, 220,000, 200,000, 180,000, 160,000, 140,000, 120,000, 100,000, 80,000, 60,000, 40,000, or 20,000 pg. Preferably, the total dose may be up to 360,000 pg of modified BoNT/A. The total dose may be at least 17,500, 20,000, 22,500, 25,000, 27,500, 30,000, 35,000, 36,000, 37,000, 38,000, 39,000, 40,000, 50,000, 60,000, 70,000, 80,000, 90,000, 100,000, 110,000, 120,000, 140,000, 160,000, 180,000, 200,000, 220,000, 240,000, 260,000, 280,000, 300,000, 320,000, 340,000, 360,000 or 380,000 pg. Preferably, the total dose may be greater than 170,000 pg, more preferably at least 240,000 pg or at least 250,000 pg of modified BoNT/A, e.g. at least 300,000 pg. The total dose may be 160,000-400,000 pg, or 170,000-400,000 pg (e.g. greater than 170,000-400,000 pg), preferably 170,000 pg up to 360,000 pg or 200,000-370,000 pg. The total dose administered may be 250,000 pg to 400,000 pg. More preferably, the total dose administered is 250,000-360,000 pg. In preferred embodiments, the total dose is 240,000, 250,000, 300,000 or 360,000 pg (e.g. 250,000 or 360,000 pg) of modified BoNT/A. In more preferred embodiments, the total dose is 300,000 or 360,000 pg (e.g. 360,000 pg).

The total dose may be 200,000 pg to 300,000 pg, such as 240,000 pg to 260,000 pg of modified BONT/A. Preferably, the total dose is up to 250,000 pg of modified BoNT/A (e.g. the total dose may be 250,000 pg of modified BoNT/A).

The total dose may be 300,000 pg to 400,000 pg, such as 350,000 pg to 370,000 pg of modified BONT/A. Preferably, the total dose is up to 360,000 pg of modified BoNT/A (e.g. the total dose may be 360,000 pg of modified BoNT/A).

Accordingly, the unit dose may be greater than 17,000 pg of modified BoNT/A and the total dose administered when carrying out the treatment regimen of the present invention may be up to 400,000 pg. In a preferable embodiment, the unit dose may be 24,000 pg and the total dose may be 240,000 pg. In another preferable embodiment, the unit dose may be 25,000 pg and the total dose may be 250,000 pg. In another preferable embodiment, the unit dose may be 30,000 pg and the total dose may be 300,000 pg. In another preferable embodiment, the unit dose may be 36,000 pg and the total dose may be 360,000 pg.

In one aspect, the invention provides a modified botulinum neurotoxin A (BoNT/A) for use in treating cervical dystonia, wherein the modified BoNT/A is administered by intramuscular injection to an affected neck muscle of a subject,
wherein the modified BoNT/A is administered by way of a unit dose of greater than 707 Units of modified BoNT/A, wherein 1 Unit is an amount of the modified BoNT/A that corresponds to the calculated median lethal dose (LD₅₀) in mice,
wherein at least a single unit dose is administered to the affected neck muscle,
wherein the total dose administered during the treatment is up to 16,639 U of modified BONT/A, and
wherein the modified BoNT/A comprises a BoNT/A light-chain and translocation domain, and a BoNT/B receptor binding domain (H_{C} domain).

In a related aspect, the invention provides a modified BoNT/A for use in treating cervical dystonia of a subject for a longer duration than that treated by an unmodified BoNT/A (e.g. SEQ ID NO: 2 [such as a di-chain form of SEQ ID NO: 2]), wherein the modified BoNT/A is administered by intramuscular injection to an affected neck muscle of the subject,
wherein the modified BoNT/A is administered by way of a unit dose of greater than 707 Units modified BoNT/A, wherein 1 Unit is an amount of the modified BoNT/A that corresponds to the calculated median lethal dose (LD₅₀) in mice,
wherein at least a single unit dose is administered to the affected neck muscle,
wherein the total dose administered during the treatment is up to 16,639 U of modified BONT/A, and
wherein the modified BoNT/A comprises a BoNT/A light-chain and translocation domain, and a BoNT/B receptor binding domain (H_{C} domain).

In one aspect, the invention provides a method for treating cervical dystonia, the method comprising administering a modified BoNT/A by intramuscular injection to an affected neck muscle of a subject,
wherein the modified BoNT/A is administered by way of a unit dose of greater than 707 U of modified BoNT/A, wherein 1 Unit is an amount of the modified BoNT/A that corresponds to the calculated median lethal dose (LD₅₀) in mice,
wherein at least a single unit dose is administered to the affected neck muscle,
wherein the total dose administered during the treatment is up to 16,639 U of modified BONT/A, and
wherein the modified BoNT/A comprises a BoNT/A light-chain and translocation domain, and a BoNT/B receptor binding domain (H_{C} domain).

In a related aspect, the invention provides a method for treating cervical dystonia of a subject for a longer duration than that treated by an unmodified BoNT/A (e.g. SEQ ID NO: 2 [such as a di-chain form of SEQ ID NO: 2]), the method comprising administering a modified BoNT/A by intramuscular injection to an affected neck muscle of the subject,
wherein the modified BoNT/A is administered by way of a unit dose of greater than 707 Units of modified BoNT/A, wherein 1 Unit is an amount of the modified BoNT/A that corresponds to the calculated median lethal dose (LD₅₀) in mice,
wherein at least a single unit dose is administered to the affected neck muscle,
wherein the total dose administered during the treatment is up to 16,639 U of modified BONT/A, and
wherein the modified BoNT/A comprises a BoNT/A light-chain and translocation domain, and a BoNT/B receptor binding domain (H_{C} domain).

In one aspect, the invention provides the use of a modified botulinum neurotoxin A (BoNT/A) in the manufacture of a medicament for treating cervical dystonia, wherein the modified BONT/A is administered by intramuscular injection to an affected neck muscle of a subject,
wherein the modified BoNT/A is administered by way of a unit dose of greater than 707 Units of modified BoNT/A, wherein 1 Unit is an amount of the modified BoNT/A that corresponds to the calculated median lethal dose (LD₅₀) in mice,
wherein at least a single unit dose is administered to the affected neck muscle,
wherein the total dose administered during the treatment is up to 16,639 U of modified BONT/A, and
wherein the modified BoNT/A comprises a BoNT/A light-chain and translocation domain, and a BoNT/B receptor binding domain (H_{C} domain).

In a related aspect, the invention provides use of a modified BoNT/A in the manufacture of a medicament for treating cervical dystonia of a subject for a longer duration than that treated by an unmodified BoNT/A (e.g. SEQ ID NO: 2 [such as a di-chain form of SEQ ID NO: 2]), wherein the modified BoNT/A is administered by intramuscular injection to an affected neck muscle of the subject,
wherein the modified BoNT/A is administered by way of a unit dose of greater than 707 Units of modified BoNT/A, wherein 1 Unit is an amount of the modified BoNT/A that corresponds to the calculated median lethal dose (LD₅₀) in mice,
wherein at least a single unit dose is administered to the affected neck muscle,
wherein the total dose administered during the treatment is up to 16,639 U of modified BoNT/A, and
wherein the modified BoNT/A comprises a BoNT/A light-chain and translocation domain, and a BoNT/B receptor binding domain (H_{C} domain).

The unit dose may be greater than 707 Units of modified BoNT/A. An upper limit of the unit dose range may be 1664, 1650, 1600, 1550, 1500, 1450, 1400, 1350, 1300, 1250, 1150, 1100, 1050, 1000, 950, 900, 850, 800 or 750 Units of modified BoNT/A, preferably the upper limit is 1500 Units. A lower limit of the unit dose range may be 728, 750, 775, 800, 825, 850, 875, 900, 925, 950, 975, 1000, 1025, 1050, 1075, 1100, 1250, 1300, 1350, 1400, 1450, 1500, 1550, 1600 or 1650 Units of modified BoNT/A, preferably the lower limit is 728 Units, or 1,040 Units. Preferably, the unit dose of modified BoNT/A is greater than 707 Units up to 1664 Units of modified BoNT/A, for example greater than 707 Units up to 1498 Units or 832 Units to 1622 Units. Most preferably a unit dose of modified BoNT/A is 915 to 1581 Units, such as 998 to 1498 Units. A unit dose of modified BoNT/A may be 1,040 Units up to 1,664 Units. In preferred embodiments, a unit dose of modified BoNT/A comprises 998, 1,248, 1,040 or 1,498 Units, e.g. 1,040 or 1,498 Units of modified BoNT/A. In more preferred embodiments, a unit dose comprises 1,248 or 1,498 Units (e.g. 1,248 Units) of modified BoNT/A.

The unit dose may be 832 Units to 1,248 Units, such as 998 Units to 1,082 Units of modified BONT/A. Most preferably, the unit dose is 1,040 Units of modified BoNT/A.

The unit dose may be 1,248 Units to 1,664 Units, such as 1,456 Units to 1,539 Units of modified BONT/A. Most preferably, the unit dose is 1,498 Units of modified BoNT/A.

A total dose administered when carrying out the treatment regimen of the present invention may be up to 16,639 Units. In other words, the total amount of modified BoNT/A administered at a given treatment session may be up to 16,639 Units. The total dose may be up to 16,000, 15,000, 14,000, 13,000, 12,000, 11,000, 10,000, 9.000, 8,000, 7,000, 6,000, 5,000, 4,000, 3,000, 2,000, 1,000 or 832 Units. Preferably, the total dose may be up to 14,975 Units of modified BoNT/A. The total dose may be at least 725, 750, 775, 800, 825, 850, 875, 900, 925, 950, 975, 1000, 1,100, 1,200, 1,300, 1,400, 1,500, 1,600, 2,000, 2,500, 3,000, 3,500, 4,000, 4,500, 5,000, 5,500, 6,000, 7,000, 8,000, 9,000, 10,000, 11,000, 12,000, 13,000, 14,000, 15,000, or 16,000 Units. The total dose may be at least 1,000, 2,000, 3,000, 4,000, 5,000, 6,000, 7,000, 8,000, 9,000, 10,000, 11,000, 12,000, 13,000, 14,000, 15,000, 16,000 or 16,300 Units. Preferably, the total dose may be greater than 7,072 Units, more preferably at least 9,983 Units, or 10,399 Units, of modified BoNT/A, e.g. at least 12,479 Units. The total dose may be 6,656-16,639 Units or 7,072-16,639 Units (e.g. greater than 7,072 Units up to 16,639 Units), preferably 7,072 up to 14,975 Units or 8,319-15,391 Units. More preferably, the total dose administered is 9,983-14,975 Units. The total dose administered may be 10,399 Units to 16,639 Units of modified BoNT/A. In preferred embodiments, the total dose is 9,983, 10,399, 12,479 or 14,975 Units (e.g. 10,399 or 14,975 Units). In more preferred embodiments, the total dose is 12,479 or 14,975 Units pg (e.g. 14,975 Units).

The total dose may be 8,319 Units to 12,479 Units, such as 9,983 Units to 10,815 Units of modified BoNT/A. Preferably, the total dose is up to 103,999 Units (e.g. the total dose may be 103,999 Units).

The total dose may be 12,479 Units to 16,639 Units, such as 14,559 Units to 15,391 Units of modified BoNT/A. Preferably, the total dose is up to 14,975 Units (e.g. the total dose may be 14,975 Units).

Accordingly, the unit dose may be greater than 707 Units of modified BoNT/A and the total dose administered when carrying out the treatment regimen of the present invention may be up to 16,639 Units. In a preferable embodiment, the unit dose may be 998 Units of modified BONT/A and the total dose may be 9,983 Units. In another preferable embodiment, the unit dose may be 1,248 Units of modified BoNT/A and the total dose may be 12,479 Units. In another preferable embodiment, the unit dose may be 1,498 Units of modified BoNT/A and the total dose may be 14,975 Units.

An “affected neck muscle” may be a neck muscle contributing to (e.g. causing) cervical dystonia and/or a symptom thereof in a subject or that contributes to (e.g. causes) cervical dystonia and/or a symptom thereof in a subject. It is not intended that the “affected neck muscle” necessarily has to be contributing to (e.g. causing) cervical dystonia and/or a symptom thereof at the time of treatment, although this is preferred. For example, the neck muscle may be one that in the past has contributed to (e.g. caused) cervical dystonia and/or a symptom thereof in the subject or that is expected to contribute to (e.g. cause) cervical dystonia and/or a symptom thereof in the subject in the future. In one embodiment two or more neck muscles (e.g. an agonist and antagonist pair of neck muscles) may contribute to (e.g. cause) cervical dystonia and/or a symptom thereof in a subject. In such cases, modified BoNT/A may be administered to the two or more neck muscles (e.g. administered to the agonist neck muscle and the antagonist neck muscle of the pair of neck muscles).

An affected neck muscle preferably contributes to (e.g. causes) cervical dystonia and/or a symptom thereof in a subject by contracting. Thus, an affected neck muscle is preferably a neck muscle of the subject that is contracted or that contracts resulting in cervical dystonia and/or a symptom thereof in the subject. Said neck muscle is preferably a neck muscle that involuntarily contracts or that has involuntarily contracted, e.g. at the time of treatment. A neck muscle may be any muscle (e.g. skeletal muscle) that is operably connected to the neck and/or head of a subject, for example any muscle that is capable of altering the head positioning of a subject (e.g. when contracted). An affected neck muscle may be one that is capable of: causing twisting of the chin of a subject towards a shoulder of the subject resulting in sideways head rotation (torticollis); causing tipping forward of the head of a subject (anterocollis); causing tipping backwards of the head of a subject (retrocollis); causing sideways tilting of the head of a subject (laterocollis); causing an anterior sagittal shift (a forward shift) of the head of a subject; and/or causing a posterior sagittal shift (a backwards shift) of the head of a subject.

An affected neck muscle may comprise the sternocleidomastoideornocleidomastoideus, the splenius capitis, the splenius cervicis, the scalene complex (e.g. the scalenus anterior and/or the scalenus medius), the trapezius (e.g. the upper trapezius and/or the trapezius pars descendens), the levator scapulae, the semispinalis capitis, or the longissimus (e.g. longissimus capitis and/or longissimus cervicis). An affected neck muscle may comprise the sternocleidomastoideolenius capitis, the splenius cervicis, the scalene complex (e.g. the scalenus anterior and/or the scalenus medius), the trapezius (e.g. the upper trapezius and/or the trapezius pars descendens), the levator scapulae, the semispinalis capitis, the longissimus (e.g. longissimus capitis and/or longissimus cervicis), the posterior paravertebrals (e.g. the scalenus posterior, scalenus medius and/or scalenus anterior, preferably the scalenus posterior), the submental complex (e.g. the digastric muscle, the geniohyoid muscle, the mylohyoid muscle, the mylohyoid boutonniere and/or the stylohyoid muscle), the linea nuchalis superior—Clavicula (lateral part), the processus spinosus C3-Th3—processus mastoideus, the processus spinosus Th3-Th5—processus transversus C1-C2, the processus transversus C3-Th6, the processus spinosus C3-Th1-linea nuchalis superior, the processus transversus Th1-Th6—processus spinosus C2-C7, the processus transversus C3-Th3—processus mastoideus, the processus transversus Th1-Th6—processus transversus C2-C6, the obliquus capitis inferior, the processus spinosus C2—processus transversus C1, the suprasternal notch and clavicula (medial part)—processus mastoideus and linea nuchalis superior, the processus transversus C1-C4—scapula (angulus superior), the processus transversus C2-C7—first rib, the processus transversus C3-C6—first rib, the longus capitis, the processus transversus C3-C6—occipital bone (basilar part), the longus colli, or the processus transversus C2-C5—atlas (anterior tubercle). An affected neck muscle may comprise the right levator scapulae, the left levator scapulae, the right trapezius, the left trapezius, the right sternocleidomastoideoeft sternocleidomastoideoght splenius capitis, the left splenius capitis, the scalenus medius, the scalenus anterior, the right semispinalis capitis, the left semispinalis capitis, the right longissimus capitis, or the left longissimus capitis. An affected neck muscle may comprise the sternocleidomastoideus (e.g. the left or right sternocleidomastoideoeft or right splenius capitis, the scalenus anterior or the scalenus medius, the left or right trapezius (e.g. the left or right upper trapezius), the left or right levator scapulae, the left or right semispinalis capitis, the longissimus (e.g. the left or right longissimus capitis and/or longissimus cervicis), the splenius cervicis, the scalene complex (e.g. the scalenus anterior and/or the scalenus medius), the posterior paravertebrals (e.g. the scalenus posterior, scalenus medius and/or scalenus anterior, preferably the scalenus posterior), the submental complex (e.g. the digastric muscle, the geniohyoid muscle, the mylohyoid muscle, the mylohyoid boutonniere and/or the stylohyoid muscle), the trapezius pars descendens, the linea nuchalis superior—Clavicula (lateral part), the processus spinosus C3-Th3—processus mastoideus, the processus spinosus Th3-Th5—processus transversus C1-C2, the processus transversus C3-Th6, the processus spinosus C3-Th1—linea nuchalis superior, the processus transversus Th1-Th6—processus spinosus C2-C7, the processus transversus C3-Th3—processus mastoideus, the processus transversus Th1-Th6—processus transversus C2-C6, the obliquus capitis inferior, the obliquus capitis superior, the processus spinosus C2—processus transversus C1, the suprasternal notch and clavicula (medial part)—processus mastoideus and linea nuchalis superior, the processus transversus C1-C4—scapula (angulus superior), the processus transversus C2-C7—first rib, the processus transversus C3-C6—first rib, the longus capitis, the processus transversus C3-C6—occipital bone (basilar part), the longus colli, the semispinalis cervicis, the rectus capitis posterior major, the rectus capitis posterior minor, the rectus capitis anterior, the multifudus, or the processus transversus C2-C5—atlas (anterior tubercle).

An affected neck muscle may comprise the sternocleidomastoideus (e.g. the left or right sternocleidomastoideolenius capitis (e.g. left or right splenius capitis), the scalenus anterior, the scalenus medius, the trapezius, (e.g. the left or right trapezius such as the left or right upper trapezius), the levator scapulae (e.g. left or right levator scapulae), the semispinalis capitis (e.g. the left or right semispinalis capitis), the semispinalis capitis pars med, the longissimus (e.g. the left or right longissimus capitis and/or longissimus cervicis), the splenius cervicis, the scalene complex (e.g. the scalenus anterior and/or the scalenus medius), the posterior paravertebrals (e.g. the scalenus posterior, scalenus medius and/or scalenus anterior, preferably the scalenus posterior), the submental complex (e.g. the digastric muscle, the geniohyoid muscle, the mylohyoid muscle, the mylohyoid boutonniere and/or the stylohyoid muscle), the trapezius pars descendens, the linea nuchalis superior—Clavicula (lateral part), the processus spinosus C3-Th3—processus mastoideus, the processus spinosus Th3-Th5—processus transversus C1-C2, the processus transversus C3-Th6, the processus spinosus C3-Th1—linea nuchalis superior, the processus transversus Th1-Th6—processus spinosus C2-C7, the processus transversus C3-Th3—processus mastoideus, the processus transversus Th1-Th6—processus transversus C2-C6, the obliquus capitis inferior, the obliquus capitis superior, the processus spinosus C2—processus transversus C1, the suprasternal notch and clavicula (medial part)—processus mastoideus and linea nuchalis superior, the processus transversus C1-C4—scapula (angulus superior), the processus transversus C2-C7—first rib, the processus transversus C3-C6—first rib, the longus capitis, the processus transversus C3-C6—occipital bone (basilar part), the longus colli, the semispinalis cervicis, the spinalis capitis, the rectus capitis posterior major, the rectus capitis posterior minor, the rectus capitis anterior, the multifudus, or the processus transversus C2-C5—atlas (anterior tubercle).

Most preferably, an affected neck muscle may comprise: M. semispinalis cervicis, M. levator scapulae, M. splenius cervicis, M. longissimus cervicis, M. trapezius (e.g. M. trapezius pars descendens), M. sternocleidomastoideomispinalis capitis, M. obliquus capitis inferior, M. longissimus capitis, M. splenius capitis, an M. scalenus (e.g. M. scalenus anterior, medius, and/or posterior), M. longus colli, or M. longus capitis. For example, an affected neck muscle be one or more selected from the group comprising or consisting of: M. semispinalis cervicis, M. levator scapulae, M. splenius cervicis, M. longissimus cervicis, M. trapezius (e.g. M. trapezius pars descendens), M. sternocleidomastoideomispinalis capitis, M. obliquus capitis inferior, M. longissimus capitis, M. splenius capitis, an M. scalenus (e.g. M. scalenus anterior, medius, and/or posterior), M. longus colli, and M. longus capitis.

A plurality of affected neck muscles treated in accordance with the invention may comprise at least one (e.g. at least two) of any of the muscles described herein.

In one embodiment, a modified BoNT/A may be administered to one or more affected neck muscle(s) selected from: the sternocleidomastoid, the splenius capitis, the splenius cervicis, the scalene complex (e.g. the scalenus anterior and/or the scalenus medius), the trapezius (e.g. the upper trapezius and/or the trapezius pars descendens), the levator scapulae, the semispinalis capitis, and the longissimus (e.g. longissimus capitis and/or longissimus cervicis). Preferably, a modified BoNT/A is administered to a plurality of affected neck muscles. For example, a modified BoNT/A may be administered to at least two (e.g. at least three, four, five, six or seven, preferably eight) affected neck muscles selected from: the sternocleidomastoid the splenius capitis, the splenius cervicis, the scalene complex (e.g. the scalenus anterior and/or the scalenus medius), the trapezius (e.g. the upper trapezius and/or the trapezius pars descendens), the levator scapulae, the semispinalis capitis, and the longissimus (e.g. longissimus capitis and/or longissimus cervicis).

In one embodiment, a modified BoNT/A may be administered to one or more affected neck muscle(s) selected from: the right levator scapulae, the left levator scapulae, the right trapezius, the left trapezius, the right sternocleidomastoideoft sternocleidomastoide right splenius capitis, the left splenius capitis, the scalenus medius, the scalenus anterior, the right semispinalis capitis, the left semispinalis capitis, the right longissimus capitis, and the left longissimus capitis.

In one embodiment, a modified BoNT/A may be administered to one or more affected neck muscle(s) selected from: the sternocleidomastoideornocleidomastoideus, the splenius capitis, the splenius cervicis, the scalene complex (e.g. the scalenus anterior and/or the scalenus medius), the trapezius (e.g. the upper trapezius and/or the trapezius pars descendens), the levator scapulae, the semispinalis capitis, the longissimus (e.g. longissimus capitis and/or longissimus cervicis), the posterior paravertebrals (e.g. the scalenus posterior, scalenus medius and/or scalenus anterior, preferably the scalenus posterior), the submental complex (e.g. the digastric muscle, the geniohyoid muscle, the mylohyoid muscle, the mylohyoid boutonniere and/or the stylohyoid muscle), the linea nuchalis superior—Clavicula (lateral part), the processus spinosus C3-Th3—processus mastoideus, the processus spinosus Th3-Th5—processus transversus C1-C2, the processus transversus C3-Th6, the processus spinosus C3-Th1—linea nuchalis superior, the processus transversus Th1-Th6—processus spinosus C2-C7, the processus transversus C3-Th3—processus mastoideus, the processus transversus Th1-Th6—processus transversus C2-C6, the obliquus capitis inferior, the processus spinosus C2—processus transversus C1, the suprasternal notch and clavicula (medial part)—processus mastoideus and linea nuchalis superior, the processus transversus C1-C4—scapula (angulus superior), the processus transversus C2-C7—first rib, the processus transversus C3-C6—first rib, the longus capitis, the processus transversus C3-C6—occipital bone (basilar part), the longus colli, and the processus transversus C2-C5—atlas (anterior tubercle). Preferably, a modified BoNT/A is administered to a plurality of affected neck muscles. For example, a modified BoNT/A may be administered to at least two (e.g. at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34 or 35) affected neck muscles selected from: the sternocleidomastoid, the sternocleidomastoideus, the splenius capitis, the splenius cervicis, the scalene complex (e.g. the scalenus anterior and/or the scalenus medius), the trapezius (e.g. the upper trapezius, and/or the trapezius pars descendens), the levator scapulae, the semispinalis capitis, the longissimus (e.g. longissimus capitis and/or longissimus cervicis), the posterior paravertebrals (e.g. the scalenus posterior, scalenus medius and/or scalenus anterior, preferably the scalenus posterior), the submental complex (e.g. the digastric muscle, the geniohyoid muscle, the mylohyoid muscle, the mylohyoid boutonniere and/or the stylohyoid muscle), the linea nuchalis superior-Clavicula (lateral part), the processus spinosus C3-Th3—processus mastoideus, the processus spinosus Th3-Th5—processus transversus C1-C2, the processus transversus C3-Th6, the processus spinosus C3-Th1—linea nuchalis superior, the processus transversus Th1-Th6—processus spinosus C2-C7, the processus transversus C3-Th3—processus mastoideus, the processus transversus Th1-Th6—processus transversus C2-C6, the obliquus capitis inferior, the processus spinosus C2—processus transversus C1, the suprasternal notch and clavicula (medial part)—processus mastoideus and linea nuchalis superior, the processus transversus C1-C4—scapula (angulus superior), the processus transversus C2-C7—first rib, the processus transversus C3-C6—first rib, the longus capitis, the processus transversus C3-C6—occipital bone (basilar part), the longus colli, and the processus transversus C2-C5—atlas (anterior tubercle).

In one embodiment, a modified BoNT/A may be administered to one or more affected neck muscle(s) comprising: the sternocleidomastoideus (e.g. the left or right sternocleidomastoid the splenius capitis (e.g. left or right splenius capitis), the scalenus anterior, the scalenus medius, the trapezius, (e.g. the left or right trapezius such as the left or right upper trapezius), the levator scapulae (e.g. left or right levator scapulae), the semispinalis capitis (e.g. the left or right semispinalis capitis), the semispinalis capitis pars med, the longissimus (e.g. the left or right longissimus capitis and/or longissimus cervicis), the splenius cervicis, the scalene complex (e.g. the scalenus anterior and/or the scalenus medius), the posterior paravertebrals (e.g. the scalenus posterior, scalenus medius and/or scalenus anterior, preferably the scalenus posterior), the submental complex (e.g. the digastric muscle, the geniohyoid muscle, the mylohyoid muscle, the mylohyoid boutonniere and/or the stylohyoid muscle), the trapezius pars descendens, the linea nuchalis superior—Clavicula (lateral part), the processus spinosus C3-Th3—processus mastoideus, the processus spinosus Th3-Th5—processus transversus C1-C2, the processus transversus C3-Th6, the processus spinosus C3-Th1—linea nuchalis superior, the processus transversus Th1-Th6—processus spinosus C2-C7, the processus transversus C3-Th3—processus mastoideus, the processus transversus Th1-Th6—processus transversus C2-C6, the obliquus capitis inferior, the obliquus capitis superior, the processus spinosus C2—processus transversus C1, the suprasternal notch and clavicula (medial part)—processus mastoideus and linea nuchalis superior, the processus transversus C1-C4—scapula (angulus superior), the processus transversus C2-C7—first rib, the processus transversus C3-C6—first rib, the longus capitis, the processus transversus C3-C6—occipital bone (basilar part), the longus colli, the semispinalis cervicis, the spinalis capitis, the rectus capitis posterior major, the rectus capitis posterior minor, the rectus capitis anterior, the multifudus, and/or the processus transversus C2-C5—atlas (anterior tubercle). Preferably, a modified BoNT/A is administered to a plurality of affected neck muscles. For example, a modified BoNT/A may be administered to at least two (e.g. at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34 or 35) affected neck muscles comprising: the sternocleidomastoideus (e.g. the left or right sternocleidomastoid, the splenius capitis (e.g. left or right splenius capitis), the scalenus anterior, the scalenus medius, the trapezius, (e.g. the left or right trapezius such as the left or right upper trapezius), the levator scapulae (e.g. left or right levator scapulae), the semispinalis capitis (e.g. the left or right semispinalis capitis), the semispinalis capitis pars med, the longissimus (e.g. the left or right longissimus capitis and/or longissimus cervicis), the splenius cervicis, the scalene complex (e.g. the scalenus anterior and/or the scalenus medius), the posterior paravertebrals (e.g. the scalenus posterior, scalenus medius and/or scalenus anterior, preferably the scalenus posterior), the submental complex (e.g. the digastric muscle, the geniohyoid muscle, the mylohyoid muscle, the mylohyoid boutonniere and/or the stylohyoid muscle), the trapezius pars descendens, the linea nuchalis superior—Clavicula (lateral part), the processus spinosus C3-Th3—processus mastoideus, the processus spinosus Th3-Th5—processus transversus C1-C2, the processus transversus C3-Th6, the processus spinosus C3-Th1—linea nuchalis superior, the processus transversus Th1-Th6—processus spinosus C2-C7, the processus transversus C3-Th3—processus mastoideus, the processus transversus Th1-Th6—processus transversus C2-C6, the obliquus capitis inferior, the obliquus capitis superior, the processus spinosus C2—processus transversus C1, the suprasternal notch and clavicula (medial part)—processus mastoideus and linea nuchalis superior, the processus transversus C1-C4—scapula (angulus superior), the processus transversus C2-C7—first rib, the processus transversus C3-C6—first rib, the longus capitis, the processus transversus C3-C6—occipital bone (basilar part), the longus colli, the semispinalis cervicis, the spinalis capitis, the rectus capitis posterior major, the rectus capitis posterior minor, the rectus capitis anterior, the multifudus, and/or the processus transversus C2-C5—atlas (anterior tubercle).

In one embodiment, a modified BoNT/A may be administered to one or more affected neck muscle(s) selected from: the sternocleidomastoide (e.g. the left or right sternocleidomastoideolenius capitis (e.g. left or right splenius capitis), the scalenus anterior, the scalenus medius, the trapezius, (e.g. the left or right trapezius such as the left or right upper trapezius), the levator scapulae (e.g. left or right levator scapulae), the semispinalis capitis (e.g. the left or right semispinalis capitis), the semispinalis capitis pars med, the longissimus (e.g. the left or right longissimus capitis and/or longissimus cervicis), the splenius cervicis, the scalene complex (e.g. the scalenus anterior and/or the scalenus medius), the posterior paravertebrals (e.g. the scalenus posterior, scalenus medius and/or scalenus anterior, preferably the scalenus posterior), the submental complex (e.g. the digastric muscle, the geniohyoid muscle, the mylohyoid muscle, the mylohyoid boutonniere and/or the stylohyoid muscle), the trapezius pars descendens, the linea nuchalis superior—Clavicula (lateral part), the processus spinosus C3-Th3—processus mastoideus, the processus spinosus Th3-Th5—processus transversus C1-C2, the processus transversus C3-Th6, the processus spinosus C3-Th1—linea nuchalis superior, the processus transversus Th1-Th6—processus spinosus C2-C7, the processus transversus C3-Th3—processus mastoideus, the processus transversus Th1-Th6—processus transversus C2-C6, the obliquus capitis inferior, the obliquus capitis superior, the processus spinosus C2—processus transversus C1, the suprasternal notch and clavicula (medial part)—processus mastoideus and linea nuchalis superior, the processus transversus C1-C4—scapula (angulus superior), the processus transversus C2-C7—first rib, the processus transversus C3-C6—first rib, the longus capitis, the processus transversus C3-C6—occipital bone (basilar part), the longus colli, the semispinalis cervicis, the spinalis capitis, the rectus capitis posterior major, the rectus capitis posterior minor, the rectus capitis anterior, the multifudus, and the processus transversus C2-C5—atlas (anterior tubercle). Preferably, a modified BoNT/A is administered to a plurality of affected neck muscles. For example, a modified BoNT/A may be administered to at least two (e.g. at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34 or 35) affected neck muscles selected from: the sternocleidomastoideus (e.g. the left or right sternocleidomastoideolenius capitis (e.g. left or right splenius capitis), the scalenus anterior, the scalenus medius, the trapezius, (e.g. the left or right trapezius such as the left or right upper trapezius), the levator scapulae (e.g. left or right levator scapulae), the semispinalis capitis (e.g. the left or right semispinalis capitis), the semispinalis capitis pars med, the longissimus (e.g. the left or right longissimus capitis and/or longissimus cervicis), the splenius cervicis, the scalene complex (e.g. the scalenus anterior and/or the scalenus medius), the posterior paravertebrals (e.g. the scalenus posterior, scalenus medius and/or scalenus anterior, preferably the scalenus posterior), the submental complex (e.g. the digastric muscle, the geniohyoid muscle, the mylohyoid muscle, the mylohyoid boutonniere and/or the stylohyoid muscle), the trapezius pars descendens, the linea nuchalis superior—Clavicula (lateral part), the processus spinosus C3-Th3—processus mastoideus, the processus spinosus Th3-Th5—processus transversus C1-C2, the processus transversus C3-Th6, the processus spinosus C3-Th1—linea nuchalis superior, the processus transversus Th1-Th6—processus spinosus C2-C7, the processus transversus C3-Th3—processus mastoideus, the processus transversus Th1-Th6—processus transversus C2-C6, the obliquus capitis inferior, the obliquus capitis superior, the processus spinosus C2—processus transversus C1, the suprasternal notch and clavicula (medial part)—processus mastoideus and linea nuchalis superior, the processus transversus C1-C4—scapula (angulus superior), the processus transversus C2-C7—first rib, the processus transversus C3-C6—first rib, the longus capitis, the processus transversus C3-C6—occipital bone (basilar part), the longus colli, the semispinalis cervicis, the spinalis capitis, the rectus capitis posterior major, the rectus capitis posterior minor, the rectus capitis anterior, the multifudus, and the processus transversus C2-C5—atlas (anterior tubercle).

Most preferably, a modified BoNT/A may be administered to one or more affected neck muscle(s) comprising: M. semispinalis cervicis, M. levator scapulae, M. splenius cervicis, M. longissimus cervicis, M. trapezius (e.g. M. trapezius pars descendens), M. sternocleidomastoideomispinalis capitis, M. obliquus capitis inferior, M. longissimus capitis, M. splenius capitis, an M. scalenus (e.g. M. scalenus anterior, medius, and/or posterior), M. longus colli, and/or M. longus capitis. For example, a modified BoNT/A may be administered to one or more affected neck muscle(s) selected from: M. semispinalis cervicis, M. levator scapulae, M. splenius cervicis, M. longissimus cervicis, M. trapezius (e.g. M. trapezius pars descendens), M. sternocleidomastoideomispinalis capitis, M. obliquus capitis inferior, M. longissimus capitis, M. splenius capitis, an M. scalenus (e.g. M. scalenus anterior, medius, and/or posterior), M. longus colli, and M. longus capitis. For example, a modified BoNT/A may be administered to at least two (e.g. at least 3, 4, 5, 6, 7, 8, 9, 10, 11, 12, 13, 14, or 15) of said affected neck muscles.

In one embodiment, a modified BoNT/A may be administered to one or more affected neck muscle(s) comprising: the right levator scapulae, the left levator scapulae, the right trapezius, the left trapezius, the right sternocleidomastoideoft sternocleidomastoideoght splenius capitis, the left splenius capitis, the scalenus medius, the scalenus anterior, the right semispinalis capitis, the left semispinalis capitis, the right longissimus capitis, and/or the left longissimus capitis. In one embodiment, a modified BoNT/A may be administered to one or more affected neck muscle(s) selected from: the right levator scapulae, the left levator scapulae, the right trapezius, the left trapezius, the right sternocleidomastoideoft sternocleidomastoideoght splenius capitis, the left splenius capitis, the scalenus medius, the scalenus anterior, the right semispinalis capitis, the left semispinalis capitis, the right longissimus capitis, and the left longissimus capitis.

Where there are two equivalent neck muscles either side of the neck (e.g. the sternocleidomastoid muscles, such as the left and right sternocleidomastoid muscles) the modified BoNT/A may be administered unilaterally (e.g. to one of the muscles where only one is contracted) or bilaterally (e.g. to both of the muscles where both muscles are contracted). When treating a plurality of affected muscles, it is preferred, that this is a plurality of different types of affected muscles. For example, where bilateral administration to two affected sternocleidomastoid muscles is carried out, it is preferred that a further muscle is treated when treating a plurality of affected muscles as described herein.

Where there are two equivalent neck muscles either side of the neck, unilateral administration may be to either of said equivalent neck muscles. For example, administration may be to a contracted muscle or a non-contracted equivalent muscle. In one embodiment, unilateral administration is to a muscle on a side of a subject's neck, wherein the side of the subject's neck exhibits a symptom of cervical dystonia or to an equivalent muscle on a contralateral side of the subject's neck, wherein the contralateral side of the subject's neck does not exhibit a symptom of cervical dystonia.

The affected neck muscles selected for treatment according to the invention may depend on the particular presentation of cervical dystonia of the subject to be treated (e.g. torticollis, laterocollis, anterocollis, retrocollis, laterocaput, torticaput, antecaput, retrocaput, lateral shift, sagittal shift or combinations thereof). Jost, W. H., & Tatu, L. (2015). Selection of Muscles for Botulinum Toxin Injections in Cervical Dystonia. Movement disorders clinical practice, 2(3), 224-226.https://doi.org/10.1002/mdc3.12172 describes typical presentations of cervical dystonia and associated muscles involved. Thus, a muscle or set of muscles treated in accordance with the invention may be a muscle or set of muscles as described in the aforementioned reference Jost & Tatu (2015), which is incorporated herein by reference.

In one embodiment, when treating torticollis, a modified BoNT/A may be administered to one or more affected neck muscles selected from: the sternocleidomastoid, the trapezius (e.g. upper trapezius), the scalenus anterior, the splenius capitis, the splenius cervicis, the levator scapulae, and the longissimus (e.g. the longissimus capitis and/or longissimus cervicis). In one embodiment, when treating torticollis, modified BoNT/A may be administered contralaterally to one or more affected neck muscles selected from: the sternocleidomastoid the trapezius (e.g. upper trapezius), and the scalenus anterior; and/or administered ipsilaterally to one or more affected neck muscles selected from: the splenius capitis, the splenius cervicis, the levator scapulae, and the longissimus (e.g. the longissimus capitis and/or longissimus cervicis).

In one embodiment, when treating torticollis, a modified BoNT/A may be administered to one or more affected neck muscles selected from: the sternocleidomastoid, the trapezius (e.g. upper trapezius), the scalenus anterior, the splenius capitis, the splenius cervicis, the levator scapulae, the longissimus (e.g. the longissimus capitis and/or longissimus cervicis) and the semispinalis cervicis. In one embodiment, when treating torticollis, modified BoNT/A may be administered contralaterally to one or more affected neck muscles selected from: the sternocleidomastoid, the trapezius (e.g. upper trapezius), and the scalenus anterior; and/or administered ipsilaterally to one or more affected neck muscles selected from: the splenius capitis, the splenius cervicis, the levator scapulae, the longissimus (e.g. the longissimus capitis and/or longissimus cervicis), and the semispinalis cervicis.

In one embodiment, when treating torticollis, a modified BoNT/A may be administered to one or more affected neck muscles selected from: the sternocleidomastoid, the trapezius (e.g. upper trapezius), the scalenus anterior, the splenius capitis, the splenius cervicis, the levator scapulae, the longissimus (e.g. the longissimus capitis and/or longissimus cervicis), the semispinalis cervicis, the rectus capitis posterior major, the multifidus and the obliquus capitis inferior. In one embodiment, when treating torticollis, modified BoNT/A may be administered contralaterally to one or more affected neck muscles selected from: the sternocleidomastoid the trapezius (e.g. upper trapezius), the scalenus anterior, the semispinalis cervicis, and the multifudus; and/or administered ipsilaterally to one or more affected neck muscles selected from: the splenius capitis, the splenius cervicis, the levator scapulae, the longissimus (e.g. the longissimus capitis and/or longissimus cervicis), the rectus capitis posterior major, and the obliquus capitis inferior. In one embodiment, when treating torticollis, a modified BoNT/A may be administered to one or more affected neck muscle(s) comprising: the sternocleidomastoid the trapezius (e.g. upper trapezius), the scalenus anterior, the splenius capitis, the splenius cervicis, the levator scapulae, the longissimus (e.g. the longissimus capitis and/or longissimus cervicis), the semispinalis cervicis, the rectus capitis posterior major, the multifidus and/or the obliquus capitis inferior. In one embodiment, when treating torticollis, modified BoNT/A may be administered contralaterally to one or more affected neck muscle(s) comprising: the sternocleidomastoid, the trapezius (e.g. upper trapezius), the scalenus anterior, the semispinalis cervicis, and/or the multifudus; and/or administered ipsilaterally to one or more affected neck muscle(s) comprising: the splenius capitis, the splenius cervicis, the levator scapulae, the longissimus (e.g. the longissimus capitis and/or longissimus cervicis), the rectus capitis posterior major, and/or the obliquus capitis inferior.

In one embodiment, when treating torticollis, a modified BoNT/A is administered to one or more affected neck muscle(s) selected from or comprising: M. semispinalis cervicis, M. levator scapulae, M. splenius cervicis, and/or M. longissimus cervicis. The modified BoNT/A may be administered ipsilaterally.

In one embodiment, when treating laterocollis, a modified BoNT/A may be administered to one or more affected neck muscles selected from: the levator scapulae, the trapezius (e.g. upper trapezius), the scalene complex (e.g. the scalenus anterior and/or the scalenus medius), the sternocleidomastoideolenius capitis, the splenius cervicis, and the longissimus (e.g. the longissimus capitis and/or longissimus cervicis).

In one embodiment, when treating laterocollis, a modified BoNT/A may be administered to one or more affected neck muscles selected from: the levator scapulae, the trapezius (e.g. upper trapezius), the scalene complex (e.g. the scalenus anterior and/or the scalenus medius), the sternocleidomastoideolenius capitis, the splenius cervicis, the longissimus (e.g. the longissimus capitis and/or longissimus cervicis) and the semispinalis cervicis.

In one embodiment, when treating laterocollis, a modified BoNT/A may be administered to one or more affected neck muscles selected from: the levator scapulae, the trapezius (e.g. upper trapezius), the scalene complex (e.g. the scalenus anterior and/or the scalenus medius), the sternocleidomastoideolenius capitis, the splenius cervicis, the longissimus (e.g. the longissimus capitis and/or longissimus cervicis), and the multifidus. In one embodiment, when treating laterocollis, a modified BoNT/A may be administered to one or more affected neck muscles comprising: the levator scapulae, the trapezius (e.g. upper trapezius), the scalene complex (e.g. the scalenus anterior and/or the scalenus medius), the sternocleidomastoide splenius capitis, the splenius cervicis, the longissimus (e.g. the longissimus capitis and/or longissimus cervicis), and/or the multifidus.

In one embodiment, when treating laterocollis, a modified BoNT/A may be administered to one or more affected neck muscles selected from: the levator scapulae, the trapezius (e.g. upper trapezius), the scalene complex (e.g. the scalenus anterior and/or the scalenus medius), the sternocleidomastoideolenius capitis, the splenius cervicis, the longissimus (e.g. the longissimus capitis and/or longissimus cervicis), the semispinalis cervicis, and the multifidus. In one embodiment, when treating laterocollis, a modified BoNT/A may be administered to one or more affected neck muscle(s) comprising: the levator scapulae, the trapezius (e.g. upper trapezius), the scalene complex (e.g. the scalenus anterior and/or the scalenus medius), the sternocleidomastoid, the splenius capitis, the splenius cervicis, the longissimus (e.g. the longissimus capitis and/or longissimus cervicis), the semispinalis cervicis, and/or the multifidus.

In one embodiment, when treating laterocollis, a modified BoNT/A is administered to one or more affected neck muscle(s) selected from or comprising: M. levator scapulae, M. semispinalis cervicis, M. scalenus medius, and/or M. longissimus cervicis.

Preferably, when treating laterocollis, modified BoNT/A may be administered ipsilaterally to one or more of said affected neck muscles.

In one embodiment, when treating anterocollis, a modified BoNT/A may be administered to one or more affected neck muscles selected from: the sternocleidomastoid, the scalenus anterior, and the scalenus medius.

In one embodiment, when treating anterocollis, a modified BoNT/A may be administered to one or more affected neck muscles selected from: the sternocleidomastoid, the scalenus anterior, the scalenus medius, the levator scapulae, the longus colli, and the submental complex (e.g. the digastric muscle, the geniohyoid muscle, the mylohyoid muscle, the mylohyoid boutonniere and/or the stylohyoid muscle).

In one embodiment, when treating anterocollis, a modified BoNT/A may be administered to one or more affected neck muscles selected from: the sternocleidomastoid, the scalenus anterior, the scalenus medius, the longus capitis, the longus colli, and the rectus capitis anterior. In one embodiment, when treating anterocollis, a modified BoNT/A may be administered to one or more affected neck muscles comprising: the sternocleidomastoide scalenus anterior, the scalenus medius, the longus capitis, the longus colli, and/or the rectus capitis anterior.

In one embodiment, when treating anterocollis, a modified BoNT/A may be administered to one or more affected neck muscles selected from: the sternocleidomastoid, the scalenus anterior, the scalenus medius, the levator scapulae, the longus colli, the submental complex (e.g. the digastric muscle, the geniohyoid muscle, the mylohyoid muscle, the mylohyoid boutonniere and/or the stylohyoid muscle), the longus capitis, and the rectus capitis anterior. In one embodiment, when treating anterocollis, a modified BoNT/A may be administered to one or more affected neck muscle(s) comprising: the sternocleidomastoid, the scalenus anterior, the scalenus medius, the levator scapulae, the longus colli, the submental complex (e.g. the digastric muscle, the geniohyoid muscle, the mylohyoid muscle, the mylohyoid boutonniere and/or the stylohyoid muscle), the longus capitis, and/or the rectus capitis anterior.

In one embodiment, when treating anterocollis, a modified BoNT/A is administered to one or more affected neck muscle(s) selected from or comprising: M. scalenus medius, M. levator scapulae, and/or M. longus colli.

Preferably, when treating anterocollis, modified BoNT/A may be administered bilaterally.

In one embodiment, when treating retrocollis, a modified BoNT/A may be administered to one or more affected neck muscles selected from: the levator scapulae, the trapezius (e.g. upper trapezius), the longissimus (e.g. the longissimus capitis and/or longissimus cervicis), the splenius capitis, the splenius cervicis, and the semispinalis capitis.

In one embodiment, when treating retrocollis, a modified BoNT/A may be administered to one or more affected neck muscles selected from: the levator scapulae, the trapezius (e.g. upper trapezius), the longissimus (e.g. the longissimus capitis and/or longissimus cervicis), the splenius capitis, the splenius cervicis, the semispinalis capitis, the semispinalis cervicis, and the posterior paravertebrals (e.g. the scalenus posterior, scalenus medius and/or scalenus anterior, preferably the scalenus posterior).

In one embodiment, when treating retrocollis, a modified BoNT/A may be administered to one or more affected neck muscles selected from: the levator scapulae, the trapezius (e.g. upper trapezius), the longissimus (e.g. the longissimus capitis and/or longissimus cervicis), the splenius capitis, the splenius cervicis, the semispinalis capitis, the semispinalis cervicis, the spinalis capitis, the rectus capitis posterior major, the rectus capitis posterior minor, and the obliquus capitis superior. In one embodiment, when treating retrocollis, a modified BoNT/A may be administered to one or more affected neck muscles comprising: the levator scapulae, the trapezius (e.g. upper trapezius), the longissimus (e.g. the longissimus capitis and/or longissimus cervicis), the splenius capitis, the splenius cervicis, the semispinalis capitis, the semispinalis cervicis, the spinalis capitis, the rectus capitis posterior major, the rectus capitis posterior minor, and/or the obliquus capitis superior.

In one embodiment, when treating retrocollis, a modified BoNT/A may be administered to one or more affected neck muscles selected from: the levator scapulae, the trapezius (e.g. upper trapezius), the longissimus (e.g. the longissimus capitis and/or longissimus cervicis), the splenius capitis, the splenius cervicis, the semispinalis capitis, the semispinalis cervicis, the posterior paravertebrals (e.g. the scalenus posterior, scalenus medius and/or scalenus anterior, preferably the scalenus posterior), the spinalis capitis, the rectus capitis posterior major, the rectus capitis posterior minor, and the obliquus capitis superior. In one embodiment, when treating retrocollis, a modified BoNT/A may be administered to one or more affected neck muscle(s) comprising: the levator scapulae, the trapezius (e.g. upper trapezius), the longissimus (e.g. the longissimus capitis and/or longissimus cervicis), the splenius capitis, the splenius cervicis, the semispinalis capitis, the semispinalis cervicis, the posterior paravertebrals (e.g. the scalenus posterior, scalenus medius and/or scalenus anterior, preferably the scalenus posterior), the spinalis capitis, the rectus capitis posterior major, the rectus capitis posterior minor, and/or the obliquus capitis superior.

In one embodiment, when treating retrocollis, a modified BoNT/A is administered to at least M. semispinalis cervicis.

Preferably, when treating retrocollis, modified BoNT/A may be administered bilaterally.

When treating the lateral shift presentation of cervical dystonia, the muscles selected for administration may be a combination of those administered when treating laterocollis (e.g. on a first side) and laterocaput (e.g. on a second side).

In one embodiment, when treating lateral shift, a modified BoNT/A may be administered to one or more affected neck muscles selected from: the levator scapulae, the trapezius (e.g. upper trapezius or trapezius pars descendens), the scalene complex (e.g. the scalenus anterior and/or the scalenus medius), the sternocleidomastoid, the splenius capitis, the splenius cervicis, the longissimus (e.g. the longissimus capitis and/or longissimus cervicis), and the semispinalis cervicis. Preferably, when treating lateral shift, modified BoNT/A may be administered to an affected neck muscle selected from: the levator scapulae, the semispinalis cervicis, the scalenus medius and the longissimus cervicis on a first side (e.g. left side) of the neck and the modified BoNT/A may be administered to an affected muscle selected from the sternocleidomastoid, the trapezius pars descendens, the splenius capitis, the semispinalis capitis, the longissimus capitis and the levator scapulae on a second side (e.g. right side) of the neck.

In one embodiment, when treating lateral shift, a modified BoNT/A may be administered to one or more affected neck muscle(s) comprising: the levator scapulae, the trapezius (e.g. upper trapezius or trapezius pars descendens), the scalene complex (e.g. the scalenus anterior and/or the scalenus medius), the sternocleidomastoideolenius capitis, the splenius cervicis, the longissimus (e.g. the longissimus capitis and/or longissimus cervicis), and/or the semispinalis cervicis. Preferably, when treating lateral shift, modified BoNT/A may be administered to one or more affected neck muscle(s) comprising: the levator scapulae, the semispinalis cervicis, the scalenus medius and/or the longissimus cervicis (e.g. on a first side (e.g. left side) of the neck) and the modified BoNT/A may be administered to one or more affected muscle(s) comprising: the sternocleidomastoid, the trapezius pars descendens, the splenius capitis, the semispinalis capitis, the longissimus capitis and/or the levator scapulae (e.g. on a second side (e.g. right side) of the neck).

In one embodiment, when treating laterocaput, a modified BoNT/A may be administered to one or more affected neck muscles selected from: the trapezius pars descendens, the sternocleidomastoideus, the longissimus capitis, the splenius capitis, the semispinalis capitis, the levator scapulae, and the posterior paravertebrals (e.g. the scalenus posterior, scalenus medius and/or scalenus anterior, preferably the scalenus posterior). In one embodiment, when treating laterocaput, a modified BoNT/A may be administered to one or more affected neck muscle(s) comprising: the trapezius pars descendens, the sternocleidomastoideus, the longissimus capitis, the splenius capitis, the semispinalis capitis, the levator scapulae, and/or the posterior paravertebrals (e.g. the scalenus posterior, scalenus medius and/or scalenus anterior, preferably the scalenus posterior).

In one embodiment, when treating laterocaput, a modified BoNT/A is administered to one or more affected neck muscle(s) selected from or comprising: M. sternocleidomastoideus, M. trapezius pars descendens, M. splenius capitis, M. semispinalis capitis, M. longissimus capitis, and/or M. levator scapulae.

Preferably, when treating laterocaput, modified BoNT/A may be administered ipsilaterally.

In one embodiment, when treating torticaput, a modified BoNT/A may be administered to one or more affected neck muscles selected from: the trapezius pars descendens, the sternocleidomastoideus, the longissimus capitis, the splenius capitis, the semispinalis capitis pars med., and the obliquus capitis inferior. For example, a modified BoNT/A may be administered contralaterally to one or more affected neck muscles selected from: the trapezius pars descendens, the sternocleidomastoideus, the semispinalis capitis pars med; and/or the modified BoNT/A may be administered ipsilaterally to one or more neck muscles selected from: the obliquus capitis inferior, the longissimus capitis, and the splenius capitis.

In one embodiment, when treating torticaput, a modified BoNT/A may be administered to one or more affected neck muscle(s) comprising: the trapezius pars descendens, the sternocleidomastoideus, the longissimus capitis, the splenius capitis, the semispinalis capitis pars med., and/or the obliquus capitis inferior. For example, a modified BoNT/A may be administered contralaterally to one or more affected neck muscle(s) comprising: the trapezius pars descendens, the sternocleidomastoideus, and/or the semispinalis capitis pars med; and/or the modified BoNT/A may be administered ipsilaterally to one or more neck muscle(s) comprising: the obliquus capitis inferior, the longissimus capitis, and/or the splenius capitis.

Preferably, when treating torticaput, modified BoNT/A may be administered ipsilaterally or contralaterally.

In one embodiment, when treating antecaput (a.k.a. anterocaput), a modified BoNT/A may be administered to one or more affected neck muscles selected from: the longus capitis, the levator scapulae and the sternocleidomastoideus. In one embodiment, when treating antecaput, a modified BoNT/A may be administered to one or more affected neck muscle(s) comprising: the longus capitis, the levator scapulae and/or the sternocleidomastoideus.

Preferably, when treating antecaput, modified BoNT/A may be administered bilaterally.

In one embodiment, when treating retrocaput, a modified BoNT/A may be administered to one or more affected neck muscles selected from: the obliquus capitis inferior, the semispinalis capitis, the trapezius pars descendens and the splenius capitis. In one embodiment, when treating retrocaput, a modified BoNT/A may be administered to one or more affected neck muscle(s) comprising: the obliquus capitis inferior, the semispinalis capitis, the trapezius pars descendens and/or the splenius capitis.

Preferably, when treating retrocaput, modified BoNT/A may be administered bilaterally.

When treating the sagittal shift presentation of cervical dystonia, the muscles selected for administration may be a combination of those administered when treating anterocollis (e.g. on a first side) and retrocaput (e.g. on a second side).

In one embodiment, when treating sagittal shift, a modified BoNT/A may be administered to one or more affected neck muscles selected from: the sternocleidomastoideus, the scalenus anterior, the scalenus medius, the levator scapulae, the longus colli, the submental complex (e.g. the digastric muscle, the geniohyoid muscle, the mylohyoid muscle, the mylohyoid boutonniere and/or the stylohyoid muscle), the obliquus capitis inferior, the semispinalis capitis, the trapezius pars descendens and the splenius capitis. Preferably, when treating sagittal shift, modified BoNT/A may be administered to an affected neck muscle selected from: the sternocleidomastoideus, the scalenus anterior, the scalenus medius, the levator scapulae, the longus colli, and the submental complex (e.g. the digastric muscle, the geniohyoid muscle, the mylohyoid muscle, the mylohyoid boutonniere and/or the stylohyoid muscle) on a first side (e.g. left side) of the neck and modified BoNT/A may be administered to an affected neck muscle selected from: the obliquus capitis inferior, the semispinalis capitis, the trapezius pars descendens and the splenius capitis on a second side (e.g. right side) of the neck.

In one embodiment, when treating sagittal shift, a modified BoNT/A may be administered to one or more affected neck muscle(s) comprising: the sternocleidomastoideus, the scalenus anterior, the scalenus medius, the levator scapulae, the longus colli, the submental complex (e.g. the digastric muscle, the geniohyoid muscle, the mylohyoid muscle, the mylohyoid boutonniere and/or the stylohyoid muscle), the obliquus capitis inferior, the semispinalis capitis, the trapezius pars descendens and/or the splenius capitis. Preferably, when treating sagittal shift, modified BoNT/A may be administered to one or more affected neck muscle(s) comprising: the sternocleidomastoideus, the scalenus anterior, the scalenus medius, the levator scapulae, the longus colli, and/or the submental complex (e.g. the digastric muscle, the geniohyoid muscle, the mylohyoid muscle, the mylohyoid boutonniere and/or the stylohyoid muscle) (e.g. on a first side (e.g. left side) of the neck) and modified BoNT/A may be administered to one or more affected neck muscle(s) comprising: the obliquus capitis inferior, the semispinalis capitis, the trapezius pars descendens and/or the splenius capitis (e.g. on a second side (e.g. right side) of the neck).

In one embodiment, when treating sagittal shift, a modified BoNT/A may be administered to one or more affected neck muscle(s) selected from or comprising: M. scalenus medius, M. levator scapulae, M. longus colli, M. obliquus capitis inferior, M. semispinalis capitis, M. trapezius pars descendens, and/or M. splenius capitis. For example, a modified BoNT/A may be administered to one or more affected neck muscle(s) selected from or comprising: M. scalenus medius, M. levator scapulae, and/or M. longus colli (e.g. on a first side of the neck), and one or more affected neck muscle(s) selected from or comprising: M. obliquus capitis inferior, M. semispinalis capitis, M. trapezius pars descendens, and/or M. splenius capitis (e.g. on a second side of the neck).

Where a muscle is listed for administration in accordance with a treatment of the invention, the invention may further comprise administering a modified BoNT/A to an additional, unlisted muscle.

A modified BoNT/A is administered by intramuscular injection at an affected neck muscle. One or more unit doses (e.g. at least two unit doses) of modified BoNT/A may be administered to an affected neck muscle. However, it is preferred that a single unit dose only is administered per affected neck muscle. Where the neck muscle is M. splenius capitis, M. longissimus cervicis, M. trapezius, M. sternocleidomastoideomispinalis capitis, or M. levator scapulae, two unit doses may be administered. In cases of bilateral administration, two unit doses may be administered at a first side of the neck and two unit doses may be administered to the corresponding muscle at a second side of the neck. Accordingly, where the neck muscle is a sternocleidomastoideus two unit doses may be administered. For example, two unit doses may be administered to the left sternocleidomastoid and/or two unit doses may be administered to the right sternocleidomastoid. Where the neck muscle is a trapezius (e.g. trapezius pars descendens) two unit doses may be administered. For example, two unit doses may be administered to the left trapezius (e.g. the left upper trapezius) and/or two unit doses may be administered to the right trapezius (e.g. the right upper trapezius).

Thus, a single unit dose of modified BoNT/A may be administered to one or more affected neck muscle(s) selected from a first group comprising: M. splenius cervicis, M. obliquus capitis inferior, M. semispinalis cervicis, M. scalenus (e.g. M. scalenus anterior, medius, and/or posterior), M. longissimus capitis, M. longus colli, and/or M. longus capitis; and/or a single or multiple unit doses (preferably multiple unit doses) of modified BoNT/A may be administered to one or more affected neck muscle(s) selected from a second group comprising: M. splenius capitis, M. longissimus cervicis, M. trapezius (e.g. M. trapezius pars descendens), M. sternocleidomastoideomispinalis capitis, and/or M. levator scapulae.

A single unit dose of modified BoNT/A may be administered to one or more affected neck muscle(s) selected from a first group comprising: M. splenius cervicis, M. obliquus capitis inferior, M. semispinalis cervicis, M. scalenus (e.g. M. scalenus anterior, medius, and/or posterior), M. longissimus capitis, M. longus colli, and/or M. longus capitis; and/or multiple unit doses (preferably two unit doses) of modified BoNT/A may be administered to one or more affected neck muscle(s) selected from a second group comprising: M. splenius capitis, M. longissimus cervicis, M. trapezius (e.g. M. trapezius pars descendens), M. sternocleidomastoideus, M. semispinalis capitis, and/or M. levator scapulae.

A unit dose may be administered to an affected neck muscle at a single injection site. Accordingly, in some embodiments, the modified BoNT/A is administered by way of a unit dose per injection site at an affected neck muscle. However, less than a unit dose may be administered at a single injection site, in which case the unit dose may be divided (equally or unequally) between two or more injection sites of the affected neck muscle. Thus, the modified BoNT/A may be administered to an affected neck muscle at two or more injection sites. In some instances, the modified BoNT/A may be administered by way of less than a unit dose per injection site at an affected neck muscle. Advantageously, this may allow a clinician to contour the muscle and/or treat particularly affected regions of the muscle by administering more modified BoNT/A at said regions when compared to less affected regions.

In one embodiment, at least 0.25, 0.5, 1, or 2 unit dose(s) may be administered per injection (e.g. per injection site). For example, 0.25, 0.5, 1, or 2 unit dose(s) may be administered per injection (e.g. per injection site). Most preferably, a unit dose is administered per injection site.

The modified BoNT/A may be administered at a dose of greater than 17,000 pg per injection site. Preferably, the modified BoNT/A is administered at a dose of 25,000 pg or 36,000 pg per injection site. The modified BoNT/A may be administered at a dose of 20,000 pg to 30,000 pg, such as 24,000 pg to 26,000 pg, e.g. 25,000 pg. The modified BoNT/A may be administered at a dose of 30,000 pg to 40,000 pg, such as 35,000 pg to 37,000, e.g. 36,000 pg.

The modified BoNT/A may be administered at a dose of greater than 707 Units per injection site. Preferably, the modified BoNT/A is administered at a dose of 1,040 Units or 1,498 Units per injection site. The modified BoNT/A may be administered at a dose of 832 Units to 1,248 Units, such as 998 Units to 1,082 Units, e.g. 1,040 Units. The modified BoNT/A may be administered at a dose of 1,248 Units to 1,664 Units, such as 1,456 Units to 1,539 Units, e.g. 1,498 Units.

The term “at least a single unit dose is administered” means at least substantially all of a single unit dose is administered. For example, a residual amount (e.g. up to 1%, 0.1% or 0.01%) of the unit dose may remain in a vial in which the modified BoNT/A has been reconstituted. However, preferably all of at least a single unit dose is administered (e.g. at one or more injection sites).

Likewise, when administering a fraction or multiple of a unit dose, this may mean that substantially all of the fraction or multiple of the unit dose is administered. For example, a residual amount (e.g. up to 1%, 0.1% or 0.01%) of the fraction or multiple of the unit dose may remain in a vial from which the modified BoNT/A has been taken (e.g. in which the modified BoNT/A has been reconstituted). However, preferably all of the fraction or multiple of the unit dose is administered (e.g. at one or more injection sites).

Potency of a modified BoNT/A for use according to the invention may be determined by a mouse LD₅₀ assay according to standard techniques. In said assay, 1 Unit is defined as an amount of the modified BoNT/A that corresponds to the calculated median lethal dose (LD₅₀) in mice. Preferably, the calculated median lethal intraperitoneal dose in mice.

Where a modified BoNT/A for use in the invention is modified BoNT/A comprising a BoNT/A light-chain and translocation domain, and a BoNT/B receptor binding domain (H_{C} domain), an amount of a modified BoNT/A that corresponds to 1 Unit in said assay is preferably 24.04 pg.

The term “up to” when used in reference to a value (e.g. up to 400,000 pg) means up to and including the value recited. Thus, as an example, reference to administering “up to 400,000 pg” of modified BoNT/A encompasses administration of 400,000 pg of modified BoNT/A as well as administration of less than 400,000 pg of modified BoNT/A.

A unit dose may be expressed in terms of an amount of modified BoNT/A, in Units of modified BONT/A, or a combination thereof.

In one embodiment, modified BoNT/A may be administered to one or more of the following neck muscles as follows at the following dosages:

| Neck Muscle | Dosage (Unit Dose) |
|---|---|
| Sternocleidomastoid | 1 × UD |
| Splenius capitis | 1 × UD |
| Splenius cervicis | 1 × UD |
| Trapezius | 1 × UD |
| Levator scapulae | 1 × UD |
| Scalenus medius | 1 × UD |
| Scalenus anterior | 1 × UD |
| Semispinalis capitis | 1 × UD |
| Longissimus | 1 × UD |
| Posterior paravertebrals | 1 × UD |
| Submental complex | 1 × UD |

In one embodiment, modified BoNT/A may be administered to one or more of the following neck muscles as follows at the following dosages:

| Neck Muscle | Dosage (Unit Dose) |
|---|---|
| Sternocleidomastoid | 1 × UD |
| Splenius capitis | 1 × UD |
| Splenius cervicis | 1 × UD |
| Trapezius | 1 × UD |
| Levator scapulae | 1 × UD |
| Scalenus medius | 1 × UD |
| Scalenus anterior | 1 × UD |
| Semispinalis capitis | 1 × UD |
| Longissimus | 1 × UD |
| Posterior paravertebrals | 1 × UD |
| Submental complex | 1 × UD (e.g. per muscle) |

A modified BoNT/A may be administered to one or more of the following neck muscles as follows at the following dosages:

| | Dosage |
|---|---|
| | (Unit |
| Neck Muscle | Dose) |
| Either the right Levator scapulae or the left Levator scapulae | 1 × UD |
| Either the right Trapezius or the left Trapezius | 1 × UD |
| Either the right Sternocleidomastoide or the left | 1 × UD |
| Sternocleidomastoid | |
| Either the right Splenius capitis or the left Splenius capitis | 1 × UD |
| Either the Scalenus medius or the Scalenus anterior | 1 × UD |
| Either the right Semispinalis capitis or the left Semispinalis | 1 × UD |
| capitis | |
| Either the right Longissimus capitis or the left Longissimus | 1 × UD |
| capitis | |

A modified BoNT/A may be administered to the following neck muscles as follows at the following dosages:

| | Dosage |
|---|---|
| | (Unit |
| Neck Muscle | Dose) |
| Either the right Levator scapulae or the left Levator scapulae | 1 × UD |
| Either the right Trapezius or the left Trapezius | 1 × UD |
| Either the right Sternocleidomastoide or the left | 1 × UD |
| Sternocleidomastoid | |
| Either the right Splenius capitis or the left Splenius capitis | 1 × UD |
| Either the Scalenus medius or the Scalenus anterior | 1 × UD |
| Either the right Semispinalis capitis or the left Semispinalis | 1 × UD |
| capitis | |
| Either the right Longissimus capitis or the left Longissimus | 1 × UD |
| capitis | |

As used herein, the terms “right” and “left” take on their normal meaning. For example, a subject's right levator scapulae will be the levator scapulae that is on the subject's right hand side, while the subject's left levator scapulae will be the levator scapulae that is on the subject's left hand side.

The total number of unit doses administered in a given treatment may be up to 10× the unit dose or up to 7× the unit dose. The total number of unit doses may be divided according to the affected neck muscles treated, for example, in one embodiment, when the number of doses to be delivered during treatment is 1× the unit dose, then only one affected neck muscle may be treated, however, if the total is 2× unit doses then two affected neck muscles may be treated. The total number of unit doses administered may be up to 9×, 8×, 7× or 6×. The total number of unit doses administered may be at least 2×, 3×, 4×, 5×, 6×, 7× the unit dose, preferably at least 2×. The total number of unit doses administered may be 1× to 10×, or 5× to 10×, preferably 7× to 10×.

The skilled person will take into consideration when a subject has recently had (or is subsequently having) additional treatment with a clostridial neurotoxin (e.g. unmodified BoNT), e.g. as part of a cosmetic treatment or treatment for a different indication. Using techniques routine in the art, the skilled person will adapt the present treatment regimen accordingly.

A modified BoNT/A of the invention preferably has a longer duration of action (e.g. an improvement in one or more symptoms of at least 5%, 10%, 25%, or 50%) when compared to unmodified BoNT/A (e.g. Dysport®). Said duration of action may be at least 1.25×, 1.5×, 1.75×, 2.0×, or 2.25× greater. The duration of action of modified BoNT/A may be between 6 and 9 months. For example, a duration of action may be at least: 4.5 months (from onset), 5.0 months, 5.5 months, 6 months, 6.5 months, 7.0 months, 7.5 months, 8.0 months, 8.5 months or 9.0 months. In particular embodiments, a duration of action may be greater than 9.0 months.

Where administration is to a plurality of affected neck muscles, preferably said administration occurs in the same treatment session.

Treatment may be repeated at an appropriate time period following administration of modified BONT/A. Given that the duration of action is approximately twice that of unmodified BoNT/A (e.g. Dysport®) there are suitably longer periods between subsequent administrations than when a subject is treated with unmodified BoNT/A (e.g. Dysport®). A subject may be re-administered a modified BoNT/A in accordance with the present invention at least 18, 20, 25 or 30 weeks following a previous administration. For example, a subject may be re-administered a modified BoNT/A in accordance with the present invention at least 18-45 weeks, preferably 20-35 weeks following a previous administration.

The efficacy of the treatment (including severity of a subject's symptom(s)) may be assessed using the Toronto Western Spasmodic Torticollis Rating Scale (TWSTRS) reviewed in Jost et al. 2013 J Neural Transm (Vienna) 120 (3): 487-496. The TWSTRS is a composite scale consisting of the TWSTRS-Severity scale, the TWSTRS-Disability scale and the TWSTRS-Pain scale. A higher score on the TWSTRS indicates more severe disease. The TWSTRS-Severity scale includes the following items: A. maximal excursion (rotation, tilt, anterocollis or retrocollis, lateral shift, sagittal shift), B. duration factor, C. effect of sensory tricks, D. shoulder elevation/anterior displacement, E. range of motion (without the aid of sensory tricks), F. time (up to 60 s that the patient is able to maintain the head within 10° of the neutral position without the use of sensory tricks). The sum of A to F amounts to a maximum score of 35 with the duration factor weighted twice. The TWSTRS-Disability scale is a six-item scale that comprises an assessment of performances of daily activities which may be possibly affected by CD: work performance (job or domestic), activities of daily living (feeding, dressing, hygiene), driving, reading, watching television, and leisure activities outside the home. Each item is rated on a 6-point scale (0=no difficulty, 5=highest degree of disability) and the sum of each item amounts to a maximum score of 30. The TWSTRS-Pain scale consists of a severity score for the patient's usual, worst, and best pain in the last week, as well as a duration component and an assessment of the contribution of pain to disability. The score range is between 0 and 20, with 20 assigned to the highest possible experienced pain.

A “subject” as used herein may be a mammal, such as a human or other mammal. Preferably “subject” means a human subject. A “subject” is preferably an adult subject, i.e. a subject at least 18 years old. The terms “subject” and “patient” are used synonymously herein. Preferably, the subject has been diagnosed with cervical dystonia.

A subject for treatment in accordance with the invention may be a subject that is unsuitable for treatment with an unmodified BoNT/A and/or with another clostridial neurotoxin. Said subject may be a subject that is resistant to treatment with an unmodified BoNT/A and/or with another clostridial neurotoxin. Resistance may arise due to development of an immune response to a clostridial neurotoxin, including production of anti-clostridial neurotoxin antibodies, by a subject.

The term “treat” or “treating” as used herein encompasses prophylactic treatment (e.g. to prevent onset of a disorder) as well as corrective treatment (treatment of a subject already suffering from a disorder). Preferably “treat” or “treating” as used herein means corrective treatment. The term “treat” or “treating” as used herein refers to the disorder and/or a symptom thereof.

Suitable modified BoNT/A polypeptides (and nucleotide sequences encoding the same, where present) are described in WO 2015/004461 A1 and WO 2017/191315, both of which are incorporated herein by reference in their entirety.

BoNT/A is one example of a clostridial neurotoxin produced by bacteria in the genus Clostridia. Other examples of such clostridial neurotoxins include those produced by C. tetani (TeNT) and by C. botulinum (BoNT) serotypes B-G and X (see WO 2018/009903 A2), as well as those produced by C. baratii and C. butyricum. Said neurotoxins are highly potent and specific and can poison neurons and other cells to which they are delivered. The clostridial toxins are some of the most potent toxins known. By way of example, botulinum neurotoxins have median lethal dose (LD₅₀) values for mice ranging from 0.5 to 5 ng/kg, depending on the serotype. Both tetanus and botulinum toxins act by inhibiting the function of affected neurons, specifically the release of neurotransmitters. While botulinum toxin acts at the neuromuscular junction and inhibits cholinergic transmission in the peripheral nervous system, tetanus toxin acts in the central nervous system.

In nature, clostridial neurotoxins (including BoNT/A) are synthesised as a single-chain polypeptide that is modified post-translationally by a proteolytic cleavage event to form two polypeptide chains joined together by a disulphide bond. Cleavage occurs at a specific cleavage site, often referred to as the activation site (e.g. activation loop), that is located between the cysteine residues that provide the inter-chain disulphide bond. It is this di-chain form that is the active form of the toxin. The two chains are termed the heavy chain (H-chain), which has a molecular mass of approximately 100 kDa, and the light chain (L-chain), which has a molecular mass of approximately 50 kDa. The H-chain comprises an N-terminal translocation component (H_{N} domain) and a C-terminal targeting component (H_{C} domain). The cleavage site is located between the L-chain and the translocation domain components. Following binding of the H_{C} domain to its target neuron and internalisation of the bound toxin into the cell via an endosome, the H_{N} domain translocates the L-chain across the endosomal membrane and into the cytosol, and the L-chain provides a protease function (also known as a non-cytotoxic protease).

Non-cytotoxic proteases act by proteolytically cleaving intracellular transport proteins known as SNARE proteins (e.g. SNAP-25, VAMP, or Syntaxin)—see Gerald K (2002) “Cell and Molecular Biology” (4th edition) John Wiley & Sons, Inc, preferably SNAP-25. The acronym SNARE derives from the term Soluble NSF Attachment Receptor, where NSF means N-ethylmaleimide-Sensitive Factor. SNARE proteins are integral to intracellular vesicle fusion, and thus to secretion of molecules via vesicle transport from a cell. The protease function is a zinc-dependent endopeptidase activity and exhibits a high substrate specificity for SNARE proteins. Accordingly, once delivered to a desired target cell, the non-cytotoxic protease is capable of inhibiting cellular secretion from the target cell. The L-chain proteases of clostridial neurotoxins are non-cytotoxic proteases that cleave SNARE proteins.

In view of the ubiquitous nature of SNARE proteins, clostridial neurotoxins such as botulinum toxin have been successfully employed in a wide range of therapies.

For further details on the genetic basis of toxin production in Clostridium botulinum and C. tetani, see Henderson et al (1997) in The Clostridia: Molecular Biology and Pathogenesis, Academic press.

As discussed above, clostridial neurotoxins are formed from two polypeptide chains, the heavy chain (H-chain), which has a molecular mass of approximately 100 kDa, and the light chain (L-chain), which has a molecular mass of approximately 50 kDa. The H-chain comprises a C-terminal targeting component (receptor binding domain or H_{C} domain) and an N-terminal translocation component (H_{N} domain).

Clostridial neurotoxin domains are described in more detail below.

Examples of L-chain reference sequences include:
Botulinum type A neurotoxin: amino acid residues 1-448
Botulinum type B neurotoxin: amino acid residues 1-440

The above-identified reference sequences should be considered a guide, as slight variations may occur according to sub-serotypes. By way of example, US 2007/0166332 (hereby incorporated by reference in its entirety) cites slightly different clostridial sequences:
Botulinum type A neurotoxin: amino acid residues M1-K448
Botulinum type B neurotoxin: amino acid residues M1-K441

The translocation domain is a fragment of the H-chain of a clostridial neurotoxin approximately equivalent to the amino-terminal half of the H-chain, or the domain corresponding to that fragment in the intact H-chain.

Examples of reference translocation domains include:
Botulinum type A neurotoxin—amino acid residues (449-871)
Botulinum type B neurotoxin—amino acid residues (441-858)

The above-identified reference sequence should be considered a guide as slight variations may occur according to sub-serotypes. By way of example, US 2007/0166332 (hereby incorporated by reference thereto) cites slightly different clostridial sequences:
Botulinum type A neurotoxin—amino acid residues (A449-K871)
Botulinum type B neurotoxin—amino acid residues (A442-S858)

In the context of the present invention, a variety of BoNT/A H_{N} regions comprising a translocation domain can be useful in aspects of the present invention. The H_{N} regions from the heavy-chain of BoNT/A are approximately 410-430 amino acids in length and comprise a translocation domain. Research has shown that the entire length of a H_{N} region from a clostridial neurotoxin heavy-chain is not necessary for the translocating activity of the translocation domain. Thus, aspects of this embodiment can include BoNT/A H_{N} regions comprising a translocation domain having a length of, for example, at least 350 amino acids, at least 375 amino acids, at least 400 amino acids or at least 425 amino acids. Other aspects of this embodiment can include BoNT/A H_{N} regions comprising a translocation domain having a length of, for example, at most 350 amino acids, at most 375 amino acids, at most 400 amino acids or at most 425 amino acids.

The term H_{N} embraces naturally-occurring BoNT/A H_{N} portions, and modified BoNT/A H_{N} portions having amino acid sequences that do not occur in nature and/or synthetic amino acid residues. Preferably, said modified BoNT/A H_{N} portions still demonstrate the above-mentioned translocation function.

Examples of clostridial neurotoxin receptor binding domain (H_{C}) reference sequences include:
BONT/A—N872-L1296
BONT/B—E859-E1291

The ˜50 kDa H_{C} domain of a clostridial neurotoxin (such as a BoNT) comprises two distinct structural features that are referred to as the H_{CC} and H_{CN} domains, each typically of ˜25 kDa. Amino acid residues involved in receptor binding are believed to be primarily located in the H_{CC} domain. The H_{C} domain of a native clostridial neurotoxin may comprise approximately 400-440 amino acid residues. This fact is confirmed by the following publications, each of which is herein incorporated in its entirety by reference thereto: Umland TC (1997) Nat. Struct. Biol. 4:788-792; Herreros J (2000) Biochem. J. 347:199-204; Halpern J (1993) J. Biol. Chem. 268:15, pp. 11188-11192; Rummel A (2007) PNAS 104:359-364; Lacey DB (1998) Nat. Struct. Biol. 5:898-902; Knapp (1998) Am. Cryst. Assoc. Abstract Papers 25:90; Swaminathan and Eswaramoorthy (2000) Nat. Struct. Biol. 7:1751-1759; and Rummel A (2004) Mol. Microbiol. 51 (3), 631-643.

Examples of (reference) H_{CN} domains include:
Botulinum type A neurotoxin—amino acid residues (872-1110)
Botulinum type B neurotoxin—amino acid residues (859-1097)

The above sequence positions may vary a little according to serotype/sub-type, and further examples of (reference) H_{CN} domains include:
Botulinum type A neurotoxin—amino acid residues (874-1110)
Botulinum type B neurotoxin—amino acid residues (861-1097)

Examples of (reference) H_{CC} domains include:
Botulinum type A neurotoxin—amino acid residues (Y1111-L1296)
Botulinum type B neurotoxin—amino acid residues (Y1098-E1291)

The L-chain and H_{N} domain (optionally including a complete or partial activation loop, e.g. a complete activation loop when the modified BoNT/A is in a single-chain form and a cleaved/partial activation loop when in a di-chain form) may be collectively referred to as an LH_{N} domain. The LH_{N} domain thus may not further comprise an H_{C} domain.

WO 2017/191315 A1 (which is incorporated herein by reference) teaches modified BoNT/As and methods for preparing and manufacturing the same. Thus, a modified BoNT/A comprising a botulinum neurotoxin A (BoNT/A) light-chain and translocation domain (BoNT/A H_{N}), and a BoNT/B receptor binding domain (H_{C} domain) for use in the present invention may be one taught in WO 2017/191315 A1.

The term “modified BoNT/A” or “chimeric neurotoxin” as used herein means a neurotoxin comprising (preferably consisting of) a clostridial neurotoxin light-chain and translocation domain (H_{N} domain) from a first clostridial neurotoxin serotype and a receptor binding domain (H_{C} domain) originating from a second different clostridial neurotoxin serotype. Specifically, a modified BoNT/A for use in the invention comprises a botulinum neurotoxin A (BoNT/A) light-chain and translocation domain (H_{N} domain), and a BoNT/B receptor binding domain (H_{C} domain). The BoNT/A LH_{N} domain of the modified BoNT/A is covalently linked to the BoNT/B H_{C} domain. The modified BoNT/A of the invention may be referred to as a chimeric botulinum neurotoxin. Said modified BoNT/A is also referred to herein as “BoNT/AB”, “mrBoNT/AB” or a “BoNT/AB chimera”.

The L-chain and H_{N} domain (optionally including a complete or partial activation loop, e.g. a complete activation loop when the modified BoNT/A is in a single-chain form and a cleaved/partial activation loop when in a di-chain form) may be collectively referred to as an LH_{N} domain. The LH_{N} domain thus does not further comprise an H_{C} domain.

The modified BoNT/A may consist essentially of a botulinum neurotoxin A (BoNT/A) light-chain and translocation domain (H_{N} domain), and a BoNT/B receptor binding domain (H_{C} domain).

The term “consist(s) essentially of” as used in this context means that the modified BoNT/A does not further comprise one or more amino acid residues that confer additional functionality to the polypeptide, e.g. when administered to a subject. In other words, a polypeptide that “consists essentially of” a botulinum neurotoxin A (BoNT/A) light-chain and translocation domain (H_{N} domain), and a BONT/B receptor binding domain (H_{C} domain) may further comprise one or more amino acid residues (to those of the botulinum neurotoxin A (BoNT/A) light-chain and translocation domain (H_{N} domain), and BoNT/B receptor binding domain (H_{C} domain)) but said one or more further amino acid residues do not confer additional functionality to the polypeptide, e.g. when administered to a subject. Additional functionality may include enzymatic activity, binding activity and/or any physiological activity whatsoever.

The modified BoNT/A may comprise non-clostridial neurotoxin sequences in addition to any clostridial neurotoxin sequences so long as the non-clostridial neurotoxin sequences do not disrupt the ability of the modified BoNT/A to achieve its therapeutic effect. Preferably, the non-clostridial neurotoxin sequence is not one having catalytic activity, e.g. enzymatic activity. In one embodiment the modified BoNT/A of the invention does not comprise a non-clostridial catalytically active domain. In one embodiment, a modified BoNT/A does not comprise a further catalytically active domain. In one embodiment, the non-clostridial sequence is not one that binds to a cellular receptor. In other words, in one embodiment, the non-clostridial sequence is not a ligand for a cellular receptor. A cellular receptor may be a proteinaceous cellular receptor, such as an integral membrane protein. Examples of cellular receptors can be found in the IUPHAR Guide to Pharmacology Database, version 2019.4, available at https://www.guidetopharmacology.org/download.jsp #db_reports. Non-clostridial neurotoxin sequences may include tags to aid in purification, such as His-tags. In one embodiment, a modified BoNT/A of the invention does not comprise a label or a site for adding a label, such as a sortase acceptor or donor site.

Preferably, a modified BoNT/A may consist of a botulinum neurotoxin A (BoNT/A) light-chain and translocation domain (H_{N} domain), and a BoNT/B receptor binding domain (H_{C} domain).

The modified BoNT/A comprises a light-chain that is capable of exhibiting non-cytotoxic protease activity and of cleaving a SNARE protein in the cytosol of a target neuron.

Cell-based and in vivo assays may be used to determine if a clostridial neurotoxin comprising an L-chain and a functional cell binding and translocation domain has non-cytotoxic protease activity. Assays such as the Digit Abduction Score (DAS) assay, the dorsal root ganglia (DRG) assay, spinal cord neuron (SCN) assay, and mouse phrenic nerve hemidiaphragm (PNHD) assay are routine in the art. A suitable assay for determining non-cytotoxic protease activity may be one described in Aoki KR, Toxicon 39:1815-1820; 2001 or Donald et al (2018), Pharmacol Res Perspect, e00446, 1-14, which are incorporated herein by reference.

When administered to a subject, a modified BoNT/A is preferably in its active di-chain form where the light-chain and heavy-chain are joined together by a disulphide bond. Where a BONT/A (e.g. modified BoNT/A) is defined herein by way of a polypeptide sequence (SEQ ID NO), an L-chain portion of the sequence (SEQ ID NO) may constitute a first chain of the di-chain clostridial neurotoxin (e.g. di-chain modified BoNT/A) and the H_{N} and H_{C} domains together may constitute a second chain of the di-chain clostridial neurotoxin (e.g. di-chain modified BoNT/A), wherein the first and second chains are joined together by a di-sulphide bond. The skilled person will appreciate that a protease may cleave at one or more positions within the activation loop of the clostridial neurotoxin (e.g. modified BoNT/A), preferably at two positions within the activation loop. Where cleavage occurs at more than one position (preferably at two positions) within the activation loop, a small fragment of the C-terminal L-chain portion of the sequence may be absent from the di-chain clostridial neurotoxin sequence (e.g. di-chain modified BoNT/A). In view of this, the sequence of the di-chain clostridial neurotoxin (e.g. di-chain modified BoNT/A) may be slightly different to that of the corresponding single-chain clostridial neurotoxin (e.g. single-chain modified BoNT/A). The small fragment may be 1-15 amino acids. In particular, in one embodiment, when Lys-C is used to covert a single-chain modified BoNT/A into a di-chain modified BoNT/A, the small fragment of the C-terminal L-chain portion of the sequence that is absent may be SEQ ID NO: 9 or 10.

Most preferably, a modified BoNT/A for use in the invention may comprise a BoNT/A light-chain and translocation domain (a BoNT/A LH_{N} domain), and a BoNT/B H_{C} domain. The BONT/A LH_{N} domain is covalently linked to the BoNT/B H_{C} domain. Said modified BoNT/A is also referred to herein as “BoNT/AB” or a “BoNT/AB chimera”.

The C-terminal amino acid residue of the LH_{N} domain may correspond to the first amino acid residue of the 3₁₀ helix separating the LH_{N} and H_{C} domains of BoNT/A, and the N-terminal amino acid residue of the H_{C} domain may correspond to the second amino acid residue of the 3₁₀ helix separating the LH_{N} and H_{C} domains in BoNT/B.

An example of an (unmodified) BoNT/A polypeptide sequence is provided as SEQ ID NO: 2.

An example of a BoNT/B polypeptide sequence is provided as SEQ ID NO: 8 (UniProt accession number B1INP5).

Reference herein to the “first amino acid residue of the 3₁₀ helix separating the LH_{N} and H_{C} domains of BoNT/A” means the N-terminal residue of the 3₁₀ helix separating the LH_{N} and H_{C} domains.

Reference herein to the “second amino acid residue of the 3₁₀ helix separating the LH_{N} and H_{C} domains of BoNT/B” means the amino acid residue following the N-terminal residue of the 3₁₀ helix separating the LH_{N} and H_{C} domains.

A “3₁₀ helix” is a type of secondary structure found in proteins and polypeptides, along with a-helices, B-sheets and reverse turns. The amino acids in a 3₁₀ helix are arranged in a right-handed helical structure where each full turn is completed by three residues and ten atoms that separate the intramolecular hydrogen bond between them. Each amino acid corresponds to a 120° turn in the helix (i.e., the helix has three residues per turn), and a translation of 2.0 Å (=0.2 nm) along the helical axis, and has 10 atoms in the ring formed by making the hydrogen bond. Most importantly, the N—H group of an amino acid forms a hydrogen bond with the C═O group of the amino acid three residues earlier; this repeated i+3→i hydrogen bonding defines a 3₁₀ helix. A 3₁₀ helix is a standard concept in structural biology with which the skilled person is familiar.

This 3₁₀ helix corresponds to four residues which form the actual helix and two cap (or transitional) residues, one at each end of these four residues. The term “3₁₀ helix separating the LH_{N} and H_{C} domains” as used herein consists of those 6 residues.

Through carrying out structural analyses and sequence alignments, a 3₁₀ helix separating the LH_{N} and H_{C} domains was identified. This 3₁₀ helix is surrounded by an a-helix at its N-terminus (i.e. at the C-terminal part of the LH_{N} domain) and by a B-strand at its C-terminus (i.e. at the N-terminal part of the H_{C} domain). The first (N-terminal) residue (cap or transitional residue) of the 3₁₀ helix also corresponds to the C-terminal residue of this a-helix.

The 3₁₀ helix separating the LH_{N} and H_{C} domains can be for example determined from publicly available crystal structures of botulinum neurotoxins, for example 3BTA (http://www.rcsb.org/pdb/explore/explore.do?structureld=3BTA) and 1EPW (http://www.rcsb.org/pdb/explore/explore.do?structureld=1EPW) for botulinum neurotoxins A1 and B1 respectively.

In silico modelling and alignment tools which are publicly available can also be used to determine the location of the 3₁₀ helix separating the LH_{N} and H_{C} domains in other neurotoxins, for example the homology modelling servers LOOPP (Learning, Observing and Outputting Protein Patterns, http://loopp.org), PHYRE (Protein Homology/analogY Recognition Engine, http://www.sbg.bio.ic.ac.uk/phyre2/) and Rosetta (https://www.rosettacommons.org/), the protein superposition server SuperPose (http://wishart.biology.ualberta.ca/superpose/), the alignment program Clustal Omega (http://www.clustal.org/omega/), and a number of other tools/services listed at the Internet Resources for Molecular and Cell Biologists (http://molbiol-tools.ca/). In particular, the region around the “H_{N}/HCN” junction may be structurally highly conserved which renders it an ideal region to superimpose different serotypes.

For example, the following methodology may be used to determine the sequence of this 3₁₀ helix in other neurotoxins:
1. The structural homology modelling tool LOOP (http://loopp.org) may be used to obtain a predicted structure of other BoNT serotypes based on the BoNT/A1 crystal structure (3BTA.pdb);
2. The structural (pdb) files thus obtained may be edited to include only the N-terminal end of the H_{CN} domain and about 80 residues before it (which are part of the H_{N} domain), thereby retaining the “H_{N}/H_{CN}” region which is structurally highly conserved;
3. The protein superposition server SuperPose (http://wishart.biology.ualberta.ca/superpose/) may be used to superpose each serotype onto the 3BTA.pdb structure;
4. The superposed pdb files may be inspected to locate the 3₁₀ helix at the start of the H_{C} domain of BoNT/A1, and corresponding residues in the other serotype may then identified.
5. The other BoNT serotype sequences may be aligned with Clustal Omega in order to check that corresponding residues were correct.

Examples of LH_{N}, H_{C} and 3₁₀ helix domains determined by this method are presented below:

| | Accession Number | | | |
|---|---|---|---|---|
| | (Plus Sequence | | | |
| Neurotoxin | Version after Decimal) | LH_{N} | H_{C} | 3₁₀ helix |
| BoNT/A1 | A5HZZ9.1 | 1-872 | 873-1296 | ⁸⁷²NIINTS⁸⁷⁷ |
| (SEQ ID | | | | |
| NO: 2) | | | | |
| BoNT/A2 | X73423.3 | 1-872 | 873-1296 | ⁸⁷²NIVNTS⁸⁷⁷ |
| BoNT/A3 | DQ185900.1 (aka | 1-872 | 873-1292 | ⁸⁷²NIVNTS⁸⁷⁷ |
| | Q3LRX9.1) | | | |
| BoNT/A4 | EU341307.1 (aka | 1-872 | 873-1296 | ⁸⁷²NITNAS⁸⁷⁷ |
| | Q3LRX8.1) | | | |
| BoNT/A5 | EU679004.1 (aka | 1-872 | 873-1296 | ⁸⁷²NIINTS⁸⁷⁷ |
| | C1IPK2.1) | | | |
| BoNT/A6 | FJ981696.1 | 1-872 | 873-1296 | ⁸⁷²NIINTS⁸⁷⁷ |
| BoNT/A7 | JQ954969.1 (aka | 1-872 | 873-1296 | ⁸⁷²NIINTS⁸⁷⁷ |
| | K4LN57.1) | | | |
| BoNT/A8 | KM233166.1 | 1-872 | 873-1297 | ⁸⁷²NITNTS⁸⁷⁷ |
| BoNT/B1 | B1INP5.1 | 1-859 | 860-1291 | ⁸⁵⁹EILNNI⁸⁶⁴ |
| (SEQ ID | | | | |
| NO: 8) | | | | |
| BoNT/B2 | AB084152.1 (aka | 1-859 | 860-1291 | ⁸⁵⁹EILNNI⁸⁶⁴ |
| | Q8GR96.1) | | | |
| BoNT/B3 | EF028400.1 (aka | 1-859 | 860-1291 | ⁸⁵⁹EILNNI⁸⁶⁴ |
| | A2I2S2.1) | | | |
| BoNT/B4 | EF051570.1 (aka | 1-859 | 860-1291 | ⁸⁵⁹EILNNI⁸⁶⁴ |
| | A2I2W0.1) | | | |
| BoNT/B5 | EF033130.1 (aka | 1-859 | 860-1291 | ⁸⁵⁹DILNNI⁸⁶⁴ |
| | A2I2U6.1) | | | |
| BoNT/B6 | AB302852.1 (aka | 1-859 | 860-1291 | ⁸⁵⁹EILNNI⁸⁶⁴ |
| | A8R089.1) | | | |
| BoNT/B7 | JQ354985.1 (aka | 1-859 | 860-1291 | ⁸⁵⁹EILNNI⁸⁶⁴ |
| | H9CNK9.1) | | | |
| BoNT/B8 | JQ964806.1 (aka | 1-859 | 860-1292 | ⁸⁵⁹EILNNI⁸⁶⁴ |
| | I6Z8G9.1) | | | |

Using structural analysis and sequence alignments, it was found that the β-strand following the 3₁₀ helix separating the LH_{N} and H_{C} domains is a conserved structure in all botulinum and tetanus neurotoxins and starts at the 8th residue when starting from the first residue of the 3₁₀ helix separating the LH_{N} and H_{C} domains (e.g., at residue 879 for BoNT/A1).

A BONT/AB chimera may comprise an LH_{N} domain from BoNT/A covalently linked to a H_{C} domain from BoNT/B, wherein the C-terminal amino acid residue of the LH_{N} domain corresponds to the eighth amino acid residue N-terminally to the B-strand located at the beginning (N-term) of the H_{C} domain of BoNT/A, and wherein the N-terminal amino acid residue of the H_{C} domain corresponds to the seventh amino acid residue N-terminally to the B-strand located at the beginning (N-term) of the H_{C} domain of BoNT/B.

A BONT/AB chimera may comprise an LH_{N} domain from BoNT/A covalently linked to a H_{C} domain from BoNT/B, wherein the C-terminal amino acid residue of the LH_{N} domain corresponds to the C-terminal amino acid residue of the α-helix located at the end (C-terminus) of the LH_{N} domain of BoNT/A, and wherein the N-terminal amino acid residue of the H_{C} domain corresponds to the amino acid residue immediately C-terminal to the C-terminal amino acid residue of the α-helix located at the end (C-terminus) of the LH_{N} domain of BoNT/B.

The rationale of the design process of the BoNT/AB chimera was to try to ensure that the secondary structure was not compromised and thereby minimise any changes to the tertiary structure and to the function of each domain. Without wishing to be bound by theory, it is hypothesized that by not disrupting the four central amino acid residues of the 3₁₀ helix in the BONT/AB chimera ensures an optimal conformation for the chimeric neurotoxin, thereby allowing for the chimeric neurotoxin to exert its functions to their full capacity. In fact, surprisingly, retaining solely the first amino acid residue of the 3₁₀ helix of the BoNT/A and the second amino acid residue of the 3₁₀ helix onwards of BoNT/B not only allows the production of soluble and functional BoNT/AB chimera, but further leads to improved properties over other BONT/AB chimeras, in particular an increased potency, an increased Safety Ratio and/or a longer duration of action (as well as increased Safety Ratio and/or duration of action when compared to unmodified BoNT/A [e.g. SEQ ID NO: 2, such as SEQ ID NO: 2 in a di-chain form]).

The BoNT/A light-chain, BoNT/A translocation domain, and/or BoNT/B H_{C} domain may be a modified BoNT/A light-chain, BoNT/A translocation domain, and/or BoNT/B H_{C} domain or a derivative thereof, including but not limited to those described below. A modified BoNT/A light-chain, BoNT/A translocation domain, and/or BoNT/B H_{C} domain or derivative may contain one or more amino acids that has been modified as compared to the native (unmodified) form of the BoNT/A light-chain, BoNT/A translocation domain, and/or BoNT/B H_{C} domain, or may contain one or more inserted amino acids that are not present in the native (unmodified) form of the BoNT/A light-chain, BoNT/A translocation domain, and/or BoNT/B H_{C} domain. By way of example, a modified BoNT/A light-chain, BoNT/A translocation domain, and/or BoNT/B H_{C} domain may have modified amino acid sequences in one or more domains relative to the native (unmodified) BoNT/A light-chain, BoNT/A translocation domain, and/or BoNT/B H_{C} domain sequence. Such modifications may modify functional aspects thereof, for example biological activity or persistence. Thus, in one embodiment, the BoNT/A light-chain, BoNT/A translocation domain, and/or BoNT/B H_{C} domain is a modified BoNT/A light-chain, BoNT/A translocation domain, and/or BoNT/B H_{C} domain, or modified BoNT/A light-chain, BoNT/A translocation domain, and/or BoNT/B H_{C} domain derivative.

A modified BoNT/B H_{C} domain may have one or more modifications modifying binding to target nerve cells, for example providing higher or lower affinity binding when compared to the native (unmodified) BoNT/B H_{C} domain. Such modifications in the BoNT/B H_{C} domain may include modifying residues in the ganglioside binding site of the H_{C} domain or in the protein (e.g. synaptotagmin) binding site that alter binding to the ganglioside receptor and/or the protein receptor of the target nerve cell. Examples of such modified neurotoxins are described in WO 2006/027207 and WO 2006/114308, both of which are hereby incorporated by reference in their entirety.

A modified light-chain may have one or more modifications in the amino acid sequence thereof, for example modifications in the substrate binding or catalytic domain which may alter or modify the SNARE protein specificity of the modified light-chain, preferably with the proviso that said modifications do not catalytically inactivate said light-chain. Examples of such modified neurotoxins are described in WO 2010/120766 and US 2011/0318385, both of which are hereby incorporated by reference in their entirety.

The LH_{N} domain from BoNT/A may correspond to amino acid residues 1 to 872 of SEQ ID NO: 2, or a polypeptide sequence having at least 70% sequence identity thereto. The LH_{N} domain from BoNT/A may correspond to amino acid residues 1 to 872 of SEQ ID NO: 2, or a polypeptide sequence having at least 80%, 90% or 95% sequence identity thereto. Preferably, the LH_{N} domain from BoNT/A corresponds to amino acid residues 1 to 872 of SEQ ID NO: 2.

The H_{C} domain from BoNT/B may correspond to amino acid residues 860 to 1291 of SEQ ID NO: 8, or a polypeptide sequence having at least 70% sequence identity thereto. The H_{C} domain from BoNT/B may correspond to amino acid residues 860 to 1291 of SEQ ID NO: 8, or a polypeptide sequence having at least 80%, 90% or 95% sequence identity thereto. Preferably, the H_{C} domain from BoNT/B corresponds to amino acid residues 860 to 1291 of SEQ ID NO: 8.

Preferably, the BoNT/AB chimera comprises a BoNT/A1 LH_{N} domain and a BoNT/B1 H_{C} domain. More preferably, the LH_{N} domain corresponds to amino acid residues 1 to 872 of BONT/A1 (SEQ ID NO: 2) and the H_{C} domain corresponds to amino acid residues 860 to 1291 of BoNT/B1 (SEQ ID NO: 8).

Most preferably, a BoNT/B H_{C} domain further comprises at least one amino acid residue substitution, insertion, indel or deletion in the H_{CC} subdomain which has the effect of increasing the binding affinity of BoNT/B neurotoxin for human Syt II as compared to the natural BoNT/B sequence. Suitable amino acid residue substitutions, insertions, indels or deletions in the BoNT/B H_{CC} subdomain have been disclosed in WO 2013/180799 and in WO 2016/154534 (both herein incorporated by reference).

A suitable amino acid residue substitution, insertion, indel or deletion in the BoNT/B H_{CC} subdomain may include substitution mutations selected from the group consisting of: V1118M; Y1183M; E1191M; E11911; E1191Q; E1191T; S1199Y; S1199F; S1199L; S1201V; E1191C, E1191V, E1191L, E1191Y, S1199W, S1199E, S1199H, W1178Y, W1178Q, W1178A, W1178S, Y1183C, Y1183P and combinations thereof.

A suitable amino acid residue substitution, insertion, indel or deletion in the BoNT/B H_{CC} subdomain may further include combinations of two substitution mutations selected from the group consisting of: E1191M and S1199L, E1191M and S1199Y, E1191M and S1199F, E1191Q and S1199L, E1191Q and S1199Y, E1191Q and S1199F, E1191M and S1199W, E1191M and W1178Q, E1191C and S1199W, E1191C and S1199Y, E1191C and W1178Q, E1191Q and S1199W, E1191V and S1199W, E1191V and S1199Y, or E1191V and W1178Q.

A suitable amino acid residue substitution, insertion, indel or deletion in the BoNT/B H_{CC} subdomain may also include a combination of three substitution mutations which are E1191M, S1199W and W1178Q.

Preferably, the amino acid residue substitution, insertion, indel or deletion in the BoNT/B H_{CC} subdomain includes a combination of two substitution mutations which are E1191M and S1199Y. Such modifications are present in BoNT/AB chimeras SEQ ID NO: 5 and SEQ ID NO: 6, for example. E1191M may correspond to position 1204 of SEQ ID NO: 6 and S1199Y may correspond to position 1212. Thus, SEQ ID NO: 6 may comprise 1204M and 1212Y.

The modification may be a modification when compared to BoNT/B shown as SEQ ID NO: 8, wherein the amino acid residue numbering is determined by alignment with SEQ ID NO: 8. As the presence of a methionine residue at position 1 of SEQ ID NO: 8 (as well as the SEQ ID NOs corresponding to modified BoNT/A polypeptides described herein) is optional, the skilled person will take the presence/absence of the methionine residue into account when determining amino acid residue numbering. For example, where SEQ ID NO: 8 includes a methionine, the position numbering will be as defined above (e.g. E1191 will be E1191 of SEQ ID NO: 8). Alternatively, where the methionine is absent from SEQ ID NO: 8 the amino acid residue numbering should be modified by −1 (e.g. E1191 will be E1190 of SEQ ID NO: 8). Accordingly, an initial methionine amino acid residue of a polypeptide sequence of the modified BONT/A may be optional or absent. Similar considerations apply when the methionine at position 1 of the other polypeptide sequences described herein is present/absent, and the skilled person will readily determine the correct amino acid residue numbering using techniques routine in the art.

A modified BoNT/A for use in the invention may comprise a polypeptide sequence having at least 70% sequence identity to a polypeptide sequence selected from SEQ ID NOs: 3-7. For example, a polypeptide sequence having at least 80%, 90%, 95% or 99.9% sequence identity to a polypeptide sequence selected from SEQ ID NOs: 3-7. Preferably, a modified BoNT/A for use in the invention may comprise (more preferably consist of) a polypeptide sequence selected from SEQ ID NOs: 3-7.

It is preferred that the modified BoNT/A comprises a polypeptide sequence having at least 70% sequence identity to SEQ ID NO: 6. For example, a polypeptide sequence having at least 80%, 90%, 95% or 99.9% sequence identity to SEQ ID NO: 6. Most preferably, a modified BoNT/A for use in the invention may comprise (more preferably consist of) SEQ ID NO: 6.

The term “deletion” as used herein refers to removal of one or more amino acid residues of a polypeptide without replacement of one or more amino acid residues at the site of deletion. Thus, where one amino acid residue has been deleted from a polypeptide sequence having x number of amino acid residues (for example), the resultant polypeptide has x-1 amino acid residues.

The term “indel” as used herein refers to deletion of one or more amino acid residues of a polypeptide and insertion at the deletion site of a different number of amino acid residues (either greater or fewer amino acid residues) when compared to the number of amino acid residues deleted. Thus, for an indel where two amino acid residues have been deleted from a polypeptide sequence having x number of amino acid residues (for example), the resultant polypeptide has x-1 amino acid residues or x+≥1 amino acid residues. The insertion and deletion can be carried out in any order, sequentially or simultaneously.

The term “substitution” as used herein refers to replacement of one or more amino acid residues with the same number of amino acid residues at the same site. Thus, for a substitution of a polypeptide sequence having x number of amino acid residues (for example), the resultant polypeptide also has x amino acid residues. Preferably, a substitution is a substitution at a single amino acid position.

The term “insertion” as used herein refers to addition of one or more amino acid residues of a polypeptide without deletion of one or more amino acid residues of the polypeptide at the site of insertion. Thus, where one amino acid residue has been inserted into a polypeptide sequence having x number of amino acid residues (for example), the resultant polypeptide has x+1 amino acid residues.

Methods for modifying proteins by substitution, insertion or deletion of amino acid residues are known in the art. By way of example, amino acid modifications may be introduced by modification of a DNA sequence encoding a BoNT/A (e.g. encoding unmodified BoNT/A). This can be achieved using standard molecular cloning techniques, for example by site-directed mutagenesis where short strands of DNA (oligonucleotides) coding for the desired amino acid(s) are used to replace the original coding sequence using a polymerase enzyme, or by inserting/deleting parts of the gene with various enzymes (e.g., ligases and restriction endonucleases). Alternatively, a modified gene sequence can be chemically synthesised. Typically a modification may be carried out by either modifying a nucleic acid encoding a native clostridial neurotoxin (or part thereof) such that the modified BoNT/A (or part thereof) encoded by the nucleic acid comprises the modification(s). Alternatively, a nucleic acid that encodes a modified clostridial neurotoxin (or part thereof) comprising the modification(s) may be synthesized.

Where a polypeptide sequence of a modified BoNT/A described herein comprises a tag, e.g. for purification, such as a His-tag, said tag is optional. Preferably, said tag is removed prior to use of the modified BoNT/A according to the invention.

As discussed above, a modified BoNT/A described herein has increased tissue retention properties that also provide increased potency and/or duration of action and can allow for increased dosages without any additional negative effects. One way in which these advantageous properties may be defined is in terms of the Safety Ratio of the modified BONT/A. In this regard, undesired effects of a clostridial toxin (caused by diffusion of the toxin away from the site of administration) can be assessed experimentally by measuring percentage bodyweight loss in a relevant animal model (e.g. a mouse, where loss of bodyweight is detected within seven days of administration). Conversely, desired on-target effects of a clostridial toxin can be assessed experimentally by Digital Abduction Score (DAS) assay, a measurement of muscle paralysis. The DAS assay may be performed by injection of 20 μl of neurotoxin, formulated in Gelatin Phosphate Buffer, into the mouse gastrocnemius/soleus complex, followed by assessment of Digital Abduction Score using the method of Aoki (Aoki KR, Toxicon 39:1815-1820; 2001). In the DAS assay, mice are suspended briefly by the tail in order to elicit a characteristic startle response in which the mouse extends its hind limbs and abducts its hind digits. Following neurotoxin injection, the varying degrees of digit abduction are scored on a five-point scale (0=normal to 4=maximal reduction in digit abduction and leg extension).

The Safety Ratio of a modified BoNT/A of the invention (or unmodified BoNT/A for comparison) may then be expressed as the ratio between the amount of toxin required for a 10% drop in a bodyweight (measured at peak effect within the first seven days after dosing in a mouse) and the amount of neurotoxin required for a DAS score of 2. High Safety Ratio scores are therefore desired and indicate a neurotoxin that is able to effectively paralyse a target muscle with little undesired off-target effects. A modified BoNT/A of the present invention has a Safety Ratio that is higher than the Safety Ratio of an equivalent unmodified (native) BoNT/A.

A high Safety Ratio is particularly advantageous in therapy because it represents an increase in the therapeutic index. In other words, this means that reduced dosages can be used compared to alternative clostridial neurotoxin therapeutics and/or that increased dosages can be used without any additional (e.g. deleterious) effects. Deleterious effects include systemic toxicity and undesired spread to adjacent muscles. The possibility to use higher doses of neurotoxin without additional effects is particularly advantageous as higher doses usually lead to a longer duration of action of the neurotoxin.

The potency of a modified BoNT/A may be expressed as the minimal dose of neurotoxin which leads to a given DAS score when administered to a mouse gastrocnemius/soleus complex, for example a DAS score of 2 (ED₅₀ dose) or a DAS score of 4. The Potency of a modified BoNT/A may be also expressed as the EC₅₀ dose in a cellular assay measuring SNARE cleavage by the neurotoxin, for example the EC₅₀ dose in a cellular assay measuring SNAP25 cleavage by a modified BoNT/A.

The duration of action of a modified BoNT/A may be expressed as the time required for retrieving a DAS score of 0 after administration of a given dose of neurotoxin, for example the minimal dose of neurotoxin leading to a DAS score of 4, to a mouse gastrocnemius/soleus complex.

Thus, in one embodiment, a modified BoNT/A of the present invention has a Safety Ratio that is greater than 7 (for example, at least 8, 9, 10, 15, 20, 25, 30, 35, 40, 45 or 50), wherein Safety Ratio is calculated as: dose of toxin required for-10% bodyweight change (pg/mouse) divided by DAS ED₅₀ (pg/mouse) [ED₅₀=dose required to produce a DAS score of 2]. For example, a modified BoNT/A may have a Safety Ratio of at least 8, 9, 10, 15, 20, 25, 30, 35, 40, 45 or 50.

In one embodiment, a modified BoNT/A of the present invention has a Safety Ratio of at least 10. In one embodiment, a modified BoNT/A of the present invention has a Safety Ratio of at least 15. Preferably, the modified BoNT/A has a Safety Ratio of at least 10 (e.g. a Safety Ratio of 10), more preferably at least 12 or 13 (e.g. 14-15). The chimeric clostridial neurotoxin may have a Safety Ratio of greater than 7 up to 50 e.g. 8-45, 10-20 or 12-15.

A modified BoNT/A for use in the invention may comprise a polypeptide sequence having at least 70% sequence identity to a polypeptide sequence selected from SEQ ID NOs: 3-7. For example, a polypeptide sequence having at least 80%, 90%, 95% or 99.9% sequence identity to a polypeptide sequence selected from SEQ ID NOs: 3-7. Preferably, a modified BoNT/A for use in the invention may comprise (more preferably consist of) a polypeptide sequence selected from SEQ ID NOs: 3-7. Of said modified BoNT/As, SEQ ID NO: 6 is preferred.

Thus, it is preferred that the modified BoNT/A comprises a polypeptide sequence having at least 70% sequence identity to SEQ ID NO: 6. More preferably, a polypeptide sequence having at least 80%, 90%, 95% or 99.9% sequence identity to SEQ ID NO: 6. Most preferably, a modified BoNT/A for use in the invention may comprise (more preferably consist of) SEQ ID NO: 6.

A di-chain modified BoNT/A of the invention may comprise an L-chain portion of a polypeptide sequence having at least 70%, 80%, 90%, 95%, 99.9%, or 100% sequence identity to any one of SEQ ID NOs: 3-7 constituting a first chain of the di-chain modified BoNT/A, and may comprise the H_{N} and H_{C} domains of a polypeptide sequence having at least 70%, 80%, 90%, 95%, 99.9%, or 100% sequence identity to any one of SEQ ID NOs: 3-7 together constituting a second chain of the di-chain modified BoNT/A, wherein the first and second chains are joined together by a di-sulphide bond.

Where cleavage occurs at more than one position (preferably at two positions) within the activation loop of a modified BoNT/A comprising a polypeptide sequence having at least 70%, 80%, 90%, 95%, 99.9%, or 100% sequence identity to any one of SEQ ID NOs: 3-7, a small fragment of the C-terminal L-chain portion of the sequence having at least 70%, 80%, 90%, 95%, 99.9%, or 100% sequence identity to any one of SEQ ID NOs: 3-7 may be absent from the di-chain modified BoNT/A. In view of this, the sequence of the di-chain modified BoNT/A (e.g. comprising a polypeptide sequence having at least 70%, 80%, 90%, 95%, 99.9%, or 100% sequence identity to any one of SEQ ID NOs: 3-7) may be slightly different to that of the corresponding single-chain modified BoNT/A comprising a polypeptide sequence having at least 70%, 80%, 90%, 95%, 99.9%, or 100% sequence identity to any one of SEQ ID NOs: 3-7. The small fragment may be 1-15 amino acids. In particular, in one embodiment, when Lys-C is used to covert a single-chain modified BoNT/A into a di-chain clostridial neurotoxin, the small fragment of the C-terminal L-chain portion of the sequence that is absent may be SEQ ID NO: 9 or 10.

Preferably, a di-chain modified BoNT/A of the invention may comprise an L-chain portion of a polypeptide sequence having at least 70%, 80%, 90%, 95%, 99.9%, or 100% sequence identity to SEQ ID NO: 6 constituting a first chain of the di-chain modified BoNT/A, and may comprise the H_{N} and H_{C} domains of a polypeptide sequence having at least 70%, 80%, 90%, 95%, 99.9%, or 100% sequence identity to SEQ ID NO: 6 together constituting a second chain of the di-chain modified BoNT/A, wherein the first and second chains are joined together by a di-sulphide bond.

Where cleavage occurs at more than one position (preferably at two positions) within the activation loop of a modified BoNT/A comprising a polypeptide sequence having at least 70%, 80%, 90%, 95%, 99.9%, or 100% sequence identity to SEQ ID NO: 6, a small fragment of the C-terminal L-chain portion of the sequence having at least 70%, 80%, 90%, 95%, 99.9%, or 100% sequence identity to SEQ ID NO: 6 may be absent from the di-chain modified BoNT/A. In view of this, the sequence of the di-chain modified BoNT/A (e.g. comprising a polypeptide sequence having at least 70%, 80%, 90%, 95%, 99.9%, or 100% sequence identity to SEQ ID NO: 6) may be slightly different to that of the corresponding single-chain modified BoNT/A comprising a polypeptide sequence having at least 70%, 80%, 90%, 95%, 99.9%, or 100% sequence identity to SEQ ID NO: 6. The small fragment may be 1-15 amino acids. In particular, in one embodiment, when Lys-C is used to covert a single-chain modified BoNT/A into a di-chain modified BoNT/A, the small fragment of the C-terminal L-chain portion of the sequence that is absent may be SEQ ID NO: 9 or 10.

In a particularly preferred embodiment, a di-chain modified BoNT/A comprises (or consists of) a light-chain comprising a polypeptide sequence having at least 70%, 80%, 90%, 95%, or 99.9% sequence identity to SEQ ID NO: 11 or 12 (preferably SEQ ID NO: 11) and a heavy-chain comprising a polypeptide sequence having at least 70%, 80%, 90%, 95%, or 99.9% sequence identity to SEQ ID NO: 13, wherein the light-chain and heavy-chain are joined together by a di-sulphide bond. More preferably, a di-chain modified BoNT/A comprises (or consists of) a light-chain comprising SEQ ID NO: 11 or 12 (preferably SEQ ID NO: 11) and a heavy-chain comprising SEQ ID NO: 13, wherein the light-chain and heavy-chain are joined together by a di-sulphide bond. Even more preferably, a di-chain modified BoNT/A comprises (or consists of) a light-chain having SEQ ID NO: 11 and a heavy-chain having SEQ ID NO: 13, wherein the light-chain and heavy-chain are joined together by a di-sulphide bond. The di-sulphide bond is preferably formed by and/or is between cysteine residue 429 of SEQ ID NO: 11 or 12 and cysteine residue 6 of SEQ ID NO: 13.

In a preferred embodiment, a modified BoNT/A of the invention does not comprise a therapeutic or diagnostic agent (e.g. a nucleic acid, protein, peptide or small molecule therapeutic or diagnostic agent) additional to the light-chain and heavy-chain. For example, in one embodiment, the modified BoNT/A may not comprise a covalently or non-covalently associated therapeutic or diagnostic agent. Thus, a modified BoNT/A of the invention preferably does not function as a delivery vehicle for a further therapeutic or diagnostic agent.

In embodiments where a modified BoNT/A described herein has a tag for purification (e.g. a His-tag) and/or a linker, said tag and/or linker are optional.

The modified BoNT/A is preferably in a non-complexed form (i.e. free from complexing proteins that are present in naturally occurring BoNT/A). Examples of such complexing proteins include a neurotoxin-associated proteins (NAP) and a nontoxic-nonhemagglutinin component (NTNH). However, it is preferred that the modified BoNT/A is a recombinant modified BoNT/A.

The modified BoNT/A of the present invention can be produced using recombinant nucleic acid technologies. Thus, in one embodiment, a modified BoNT/A (as described herein) is a recombinant modified BoNT/A.

In one embodiment a nucleic acid (for example, DNA) comprising a nucleic acid sequence encoding a modified BoNT/A is provided. In one embodiment, the nucleic acid sequence is prepared as part of a DNA vector comprising a promoter and a terminator. The nucleic acid sequence may be selected from any of the nucleic acid sequences described herein.

In a preferred embodiment, the vector has a promoter selected from:

| Promoter | Induction Agent | Typical Induction Condition | |
|---|---|---|---|
| Tac (hybrid) | IPTG | 0.2 mM | (0.05-2.0 mM) |
| AraBAD | L-arabinose | 0.2% | (0.002-0.4%) |
| T7-lac operator | IPTG | 0.2 mM | (0.05-2.0 mM) |

In another preferred embodiment, the vector has a promoter selected from:

| Promoter | Induction Agent | Typical Induction Condition | |
|---|---|---|---|
| Tac (hybrid) | IPTG | 0.2 mM | (0.05-2.0 mM) |
| AraBAD | L-arabinose | 0.2% | (0.002-0.4%) |
| T7-lac operator | IPTG | 0.2 mM | (0.05-2.0 mM) |
| T5-lac operator | IPTG | 0.2 mM | (0.05-2.0 mM) |

The nucleic acid molecules may be made using any suitable process known in the art. Thus, the nucleic acid molecules may be made using chemical synthesis techniques. Alternatively, the nucleic acid molecules of the invention may be made using molecular biology techniques.

The DNA construct of the present invention is preferably designed in silico, and then synthesised by conventional DNA synthesis techniques.

The above-mentioned nucleic acid sequence information is optionally modified for codon-biasing according to the ultimate host cell (e.g. E. coli) expression system that is to be employed.

The terms “nucleotide sequence” and “nucleic acid” are used synonymously herein. Preferably the nucleotide sequence is a DNA sequence.

A modified BoNT/A of the invention may be present as a single-chain or as a di-chain. However, it is preferred that the modified BoNT/A is present as a di-chain in which the L-chain is linked to the H-chain (or component thereof, e.g. the H_{N} domain) via a di-sulphide bond.

Production of a single-chain modified BoNT/A having a light-chain and a heavy-chain may be achieved using a method comprising expressing a nucleic acid encoding a modified BoNT/A in an expression host, lysing the host cell to provide a host cell homogenate containing the single-chain modified BoNT/A, and isolating the single-chain modified BoNT/A. The single-chain modified BoNT/A described herein may be proteolytically processed using a method comprising contacting a single-chain modified BoNT/A with a protease (e.g. Lys-C) that hydrolyses a peptide bond in the activation loop of the modified BoNT/A, thereby converting the single-chain modified BoNT/A into a corresponding di-chain modified BoNT/A (e.g, wherein the light-chain and heavy-chain are joined together by a disulphide bond). A di-chain modified BoNT/A is preferably obtainable by such a method.

Thus, a modified BoNT/A used in the invention is preferably a di-chain modified BoNT/A that has been produced from a single-chain BoNT/A, wherein the single-chain BoNT/A comprises or consists of a polypeptide sequence described herein. For example, it is preferred that the modified BoNT/A used in the invention is a di-chain modified BoNT/A that has been produced from a polypeptide comprising a polypeptide sequence having at least 70% (e.g. at least 80%, 90%, 95% or 99.9%) sequence identity to SEQ ID NO: 6. Most preferably, the modified BoNT/A used in the invention is a di-chain modified BoNT/A that has been produced from a polypeptide comprising (even more preferably consisting of) SEQ ID NO: 6. Accordingly, in some embodiments, the modified BoNT/A is a di-chain modified BoNT/A in which the light-chain (L-chain) is linked to the heavy-chain (H-chain) via a di-sulphide bond obtainable by a method comprising contacting a single-chain modified BoNT/A comprising SEQ ID NO: 6 with a protease that hydrolyses a peptide bond in the activation loop thereof, thereby converting the single-chain modified BoNT/A into the corresponding di-chain modified BoNT/A. In some embodiments, the modified BoNT/A is a di-chain modified BoNT/A in which the L-chain is linked to the H-chain via a di-sulphide bond obtainable by a method comprising contacting a single-chain modified BoNT/A consisting of SEQ ID NO: 6 with a protease that hydrolyses a peptide bond in the activation loop thereof, thereby converting the single-chain modified BoNT/A into the corresponding di-chain modified BoNT/A.

The protease used to cleave the activation loop is preferably Lys-C. Suitable proteases and method for cleaving activation loops to produce di-chain clostridial neurotoxins are taught in WO 2014/080206, WO2014/079495, and EP2677029A2, which are incorporated herein by reference. Lys-C may cleave an activation loop C-terminal to one or more of the lysine residues present therein. Where Lys-C cleaves the activation loop more than once, the skilled person will appreciate that a small peptide of the activation loop of a di-chain modified BoNT/A may be absent when compared to a SEQ ID NO shown herein.

The term “obtainable” as used herein also encompasses the term “obtained”. In one embodiment the term “obtainable” means obtained.

The term “one or more” as used herein may mean at least 2, 3, 4, 5, 6, 7, 8, 9, 10, 15, or 20. In one embodiment, wherein “one or more” precedes a list, “one or more” may mean all of the members of the list. Similarly, the term “at least one” as used herein may mean at least 2, 3, 4, 5, 6, 7, 8, 9, 10, 15, or 20. In one embodiment, wherein “at least one” precedes a list, “at least one” may mean all of the members of the list.

The term “disorder” as used herein also encompasses a “disease”. In one embodiment the disorder is a disease.

The modified BoNT/A of the invention may be formulated in any suitable manner for administration to a subject, for example as part of a pharmaceutical composition. Such a pharmaceutical composition may comprise a modified BoNT/A of the invention and a pharmaceutically acceptable carrier, excipient, adjuvant, propellant and/or salt.

The modified BoNT/A of the present invention may be formulated for oral, parenteral, continuous infusion, inhalation or topical application. Compositions suitable for injection may be in the form of solutions, suspensions or emulsions, or dry powders which are dissolved or suspended in a suitable vehicle prior to use.

In the case of a modified BoNT/A that is to be delivered locally, the modified BoNT/A may be formulated as a cream (e.g. for topical application), or for sub-dermal injection.

Local delivery means may include an aerosol, or other spray (e.g. a nebuliser). In this regard, an aerosol formulation of a modified BoNT/A enables delivery to the lungs and/or other nasal and/or bronchial or airway passages.

Fluid dosage forms are typically prepared utilising the modified BoNT/A and a pyrogen-free sterile vehicle. The modified BoNT/A, depending on the vehicle and concentration used, can be either dissolved or suspended in the vehicle. In preparing solutions the modified BoNT/A can be dissolved in the vehicle, the solution being made isotonic if necessary by addition of sodium chloride and sterilised by filtration through a sterile filter using aseptic techniques before filling into suitable sterile vials or ampoules and sealing. Alternatively, if solution stability is adequate, the solution in its sealed containers may be sterilised by autoclaving. Advantageously additives such as buffering, solubilising, stabilising, preservative or bactericidal, suspending or emulsifying agents and or local anaesthetic agents may be dissolved in the vehicle.

Dry powders, which are dissolved or suspended in a suitable vehicle prior to use, may be prepared by filling pre-sterilised ingredients into a sterile container using aseptic technique in a sterile area. Alternatively the ingredients may be dissolved into suitable containers using aseptic technique in a sterile area. The product is then freeze dried and the containers are sealed aseptically.

Parenteral suspensions, suitable for an administration route described herein, are prepared in substantially the same manner, except that the sterile components are suspended in the sterile vehicle, instead of being dissolved and sterilisation cannot be accomplished by filtration. The components may be isolated in a sterile state or alternatively it may be sterilised after isolation, e.g. by gamma irradiation.

Advantageously, a suspending agent for example polyvinylpyrrolidone is included in the composition(s) to facilitate uniform distribution of the components.

Administration in accordance with the present invention may take advantage of a variety of delivery technologies including microparticle encapsulation, or high-pressure aerosol impingement.

In one aspect, the invention provides a unit dosage form of modified BoNT/A (e.g. for treating cervical dystonia), the unit dosage form comprising:
a. greater than 707 Units of modified BoNT/A, wherein 1 Unit is an amount of the modified BoNT/A that corresponds to the calculated median lethal dose (LD₅₀) in mice; or
b. greater than 17,000 pg of modified BoNT/A; and
c. optionally a pharmaceutically acceptable carrier, excipient, adjuvant, and/or salt,
   wherein the modified BoNT/A comprises a BoNT/A light-chain and translocation domain, and a BoNT/B receptor binding domain (H_{C} domain)

It is preferred that the modified BoNT/A of the unit dosage form comprises a polypeptide sequence having at least 70% sequence identity to SEQ ID NO: 6. For example, a polypeptide sequence having at least 80%, 90%, 95% or 99.9% sequence identity to SEQ ID NO: 6. Most preferably, a modified BoNT/A may comprise (more preferably consist of) SEQ ID NO: 6.

A unit dosage form may comprise greater than 707 Units of modified BoNT/A. An upper limit of said range may be 1664, 1650, 1600, 1550, 1500, 1450, 1400, 1350, 1300, 1250, 1150, 1100, 1050, 1000, 950, 900, 850, 800 or 750 Units of modified BoNT/A, preferably the upper limit is 1500 Units. A lower limit of said range may be 728, 750, 775, 800, 825, 850, 875, 900, 925, 950, 975, 1000, 1025, 1050, 1075, 1100, 1250, 1300, 1350, 1400, 1450, 1500, 1550, 1600 or 1650 Units of modified BoNT/A, preferably the lower limit is 728 Units or 1,040 Units. Preferably, a unit dosage form comprises greater than 707 Units up to 1664 Units of modified BoNT/A, for example greater than 707 Units up to 1498 Units, 832 Units to 1622 Units. Most preferably a unit dosage form comprises 915 to 1581 Units of modified BoNT/A, such as 998 to 1498 Units. The unit dosage form may comprise 10,399 Units to 16,639 Units of modified BONT/A. The unit dosage form may comprise 1,040 Units up to 1,664 Units of modified BoNT/A. In preferred embodiments, a unit dosage form of modified BoNT/A comprises 998, 1,248, 1,040 or 1,498 Units, e.g. 1,040 or 1,498 Units of modified BoNT/A. In more preferred embodiments, a unit dosage form comprises 1,248 or 1,498 Units (e.g. 1,248 Units) of modified BONT/A.

The unit dosage form may comprise 832 Units to 1,248 Units, such as 998 Units to 1,082 Units of modified BoNT/A. Most preferably, the unit dosage form may comprise 1,040 Units of modified BoNT/A.

The unit dosage form may comprise 1,248 Units to 1,664 Units, such as 1,456 Units to 1,539 Units of modified BoNT/A. Most preferably, the unit dosage form may comprise 1,498 Units of modified BoNT/A.

A unit dosage form may comprise greater than 17,000 pg of modified BoNT/A. An upper limit of said range may be 40,000, 39,000, 38,000, 37,000, 36,000, 35,000, 30,000, 25,000, 24,000, 22,000, 20,000, or 18,000, pg of modified BoNT/A, preferably the upper limit is 38,000 pg. A lower limit of said range may be 17,500, 18,000, 20,000, 22,000, 24,000, 25,000, 26,000, 27,000, 28,000, 29,000, 30,000, 35,000, 36,000, 37,000, 38,000 or 39,000 pg of modified BoNT/A, preferably the lower limit is 17,500 pg or 25,000 pg. Preferably, a unit dosage form comprises greater than 17,000 pg up to 40,000 pg, e.g. greater than 17,000 pg up to 36,000 pg, 20,000 pg to 39,000 pg, of modified BoNT/A. Most preferably a unit dosage form comprises 22,000 to 38,000 pg, such as 24,000 to 36,000 pg or 25,000 to 36,000 pg of modified BoNT/A. The unit dosage form may comprise 25,000 pg up to 40,000 pg of modified BoNT/A. In preferred embodiments, a unit dosage form comprises 24,000, 25,000, 30,000 or 36,000 pg, e.g. 25,000 or 36,000 pg of modified BoNT/A. In more preferred embodiments, a unit dosage form comprises 30,000 or 36,000 ng (e.g. 36,000 pg) of modified BoNT/A.

The unit dosage form may comprise 20,000 pg to 30,000 pg, such as 24,000 pg to 26,000 pg of modified BoNT/A. Most preferably, the unit dosage form may comprise 25,000 pg of modified BONT/A.

The unit dosage form may comprise 30,000 pg to 40,000 pg, such as 35,000 pg to 37,000 pg of modified BoNT/A. Most preferably, the unit dosage form may comprise 36,000 pg of modified BONT/A.

The unit dosage form is preferably provided as a dry powder.

In another aspect, the invention provides a kit comprising:
a. the unit dosage form according to the present invention; and
b. instructions for use of the same in treating cervical dystonia; and
C. optionally a diluent.

Embodiments related to the various therapeutic uses of the invention can be applied to the methods of the invention, the unit dosage forms, and the kits, and vice versa.

### Sequence Homology

Any of a variety of sequence alignment methods can be used to determine percent identity, including, without limitation, global methods, local methods and hybrid methods, such as, e.g., segment approach methods. Protocols to determine percent identity are routine procedures within the scope of one skilled in the art. Global methods align sequences from the beginning to the end of the molecule and determine the best alignment by adding up scores of individual residue pairs and by imposing gap penalties. Non-limiting methods include, e.g., CLUSTAL W, see, e.g., Julie D. Thompson et al., CLUSTAL W: Improving the Sensitivity of Progressive Multiple Sequence Alignment Through Sequence Weighting, Position-Specific Gap Penalties and Weight Matrix Choice, 22 (22) Nucleic Acids Research 4673-4680 (1994); and iterative refinement, see, e.g., Osamu Gotoh, Significant Improvement in Accuracy of Multiple Protein. Sequence Alignments by Iterative Refinement as Assessed by Reference to Structural Alignments, 264 (4) J. Mol. Biol. 823-838 (1996). Local methods align sequences by identifying one or more conserved motifs shared by all of the input sequences. Non-limiting methods include, e.g., Match-box, see, e.g., Eric Depiereux and Ernest Feytmans, Match-Box: A Fundamentally New Algorithm for the Simultaneous Alignment of Several Protein Sequences, 8 (5) CABIOS 501-509 (1992); Gibbs sampling, see, e.g., C. E. Lawrence et al., Detecting Subtle Sequence Signals: A Gibbs Sampling Strategy for Multiple Alignment, 262 (5131) Science 208-214 (1993); Align-M, see, e.g., Ivo Van Walle et al., Align-M-A New Algorithm for Multiple Alignment of Highly Divergent Sequences, 20 (9) Bioinformatics: 1428-1435 (2004).

Thus, percent sequence identity is determined by conventional methods. See, for example, Altschul et al., Bull. Math. Bio. 48:603-16, 1986 and Henikoff and Henikoff, Proc. Natl. Acad. Sci. USA 89:10915-19, 1992. Briefly, two amino acid sequences are aligned to optimize the alignment scores using a gap opening penalty of 10, a gap extension penalty of 1, and the “blosum 62” scoring matrix of Henikoff and Henikoff (ibid.) as shown below (amino acids are indicated by the standard one-letter codes); preferably this method is used to align a sequence with a subject sequence herein (e.g. SEQ ID NO: 2) to define amino acid position numbering as described herein.

The “percent sequence identity” between two or more nucleic acid or amino acid sequences is a function of the number of identical positions shared by the sequences. Thus, % identity may be calculated as the number of identical nucleotides/amino acids divided by the total number of nucleotides/amino acids, multiplied by 100. Calculations of % sequence identity may also take into account the number of gaps, and the length of each gap that needs to be introduced to optimize alignment of two or more sequences. Sequence comparisons and the determination of percent identity between two or more sequences can be carried out using specific mathematical algorithms, such as BLAST, which will be familiar to a skilled person.

ALIGNMENT SCORES FOR DETERMINING SEQUENCE IDENTITY

| | A | R | N | D | C | Q | E | G | H | I | L | K | M | F | P | S | T | W | Y | V |
|---|---|---|---|---|---|---|---|---|---|---|---|---|---|---|---|---|---|---|---|---|
| A | 4 | | | | | | | | | | | | | | | | | | | |
| R | −1 | 5 | | | | | | | | | | | | | | | | | | |
| N | −2 | 0 | 6 | | | | | | | | | | | | | | | | | |
| D | −2 | −2 | 1 | 6 | | | | | | | | | | | | | | | | |
| C | 0 | −3 | −3 | −3 | 9 | | | | | | | | | | | | | | | |
| Q | −1 | 1 | 0 | 0 | −3 | 5 | | | | | | | | | | | | | | |
| E | −1 | 0 | 0 | 2 | −4 | 2 | 5 | | | | | | | | | | | | | |
| G | 0 | −2 | 0 | −1 | −3 | −2 | −2 | 6 | | | | | | | | | | | | |
| H | −2 | 0 | 1 | −1 | −3 | 0 | 0 | −2 | 8 | | | | | | | | | | | |
| I | −1 | −3 | −3 | −3 | −1 | −3 | −3 | −4 | −3 | 4 | | | | | | | | | | |
| L | −1 | −2 | −3 | −4 | −1 | −2 | −3 | −4 | −3 | 2 | 4 | | | | | | | | | |
| K | −1 | 2 | 0 | −1 | −3 | 1 | 1 | −2 | −1 | −3 | −2 | 5 | | | | | | | | |
| M | −1 | −1 | −2 | −3 | −1 | 0 | −2 | −3 | −2 | 1 | 2 | −1 | 5 | | | | | | | |
| F | −2 | −3 | −3 | −3 | −2 | −3 | −3 | −3 | −1 | 0 | 0 | −3 | 0 | 6 | | | | | | |
| P | −1 | −2 | −2 | −1 | −3 | −1 | −1 | −2 | −2 | −3 | −3 | −1 | −2 | −4 | 7 | | | | | |
| S | 1 | −1 | 1 | 0 | −1 | 0 | 0 | 0 | −1 | −2 | −2 | 0 | −1 | −2 | −1 | 4 | | | | |
| T | 0 | −1 | 0 | −1 | −1 | −1 | −1 | −2 | −2 | −1 | −1 | −1 | −1 | −2 | −1 | 1 | 5 | | | |
| W | −3 | −3 | −4 | −4 | −2 | −2 | −3 | −2 | −2 | −3 | −2 | −3 | −1 | 1 | −4 | −3 | −2 | 11 | | |
| Y | −2 | −2 | −2 | −3 | −2 | −1 | −2 | −3 | 2 | −1 | −1 | −2 | −1 | 3 | −3 | −2 | −2 | 2 | 7 | |
| V | 0 | −3 | −3 | −3 | −1 | −2 | −2 | −3 | −3 | 3 | 1 | −2 | 1 | −1 | −2 | −2 | 0 | −3 | −1 | 4 |

The percent identity is then calculated as:

$$\frac{Total number of identical matches}{[ length of the longer sequence plus the number of gaps introduced into the longer sequence in order to align the two sequences ]} \times 100$$

Substantially homologous polypeptides are characterized as having one or more amino acid substitutions, deletions or additions. These changes are preferably of a minor nature, that is conservative amino acid substitutions (see below) and other substitutions that do not significantly affect the folding or activity of the polypeptide; small deletions, typically of one to about 30 amino acids; and small amino- or carboxyl-terminal extensions, such as an amino-terminal methionine residue, a small linker peptide of up to about 20-25 residues, or an affinity tag.

### Conservative Amino Acid Substitutions

Basic:
   arginine
   lysine
   histidine
Acidic: glutamic acid
   aspartic acid
Polar: glutamine
   asparagine
Hydrophobic: leucine
   isoleucine
   valine
Aromatic: phenylalanine
   tryptophan
   tyrosine
Small: glycine
   alanine
   serine
   threonine
   methionine

In addition to the 20 standard amino acids, non-standard amino acids (such as 4-hydroxyproline, 6-N-methyl lysine, 2-aminoisobutyric acid, isovaline and α-methyl serine) may be substituted for amino acid residues of the polypeptides of the present invention. A limited number of non-conservative amino acids, amino acids that are not encoded by the genetic code, and unnatural amino acids may be substituted for polypeptide amino acid residues. The polypeptides of the present invention can also comprise non-naturally occurring amino acid residues.

Non-naturally occurring amino acids include, without limitation, trans-3-methylproline, 2,4-methano-proline, cis-4-hydroxyproline, trans-4-hydroxy-proline, N-methylglycine, allo-threonine, methyl-threonine, hydroxy-ethylcysteine, hydroxyethylhomo-cysteine, nitro-glutamine, homoglutamine, pipecolic acid, tert-leucine, norvaline, 2-azaphenylalanine, 3-azaphenyl-alanine, 4-azaphenyl-alanine, and 4-fluorophenylalanine. Several methods are known in the art for incorporating non-naturally occurring amino acid residues into proteins. For example, an in vitro system can be employed wherein nonsense mutations are suppressed using chemically aminoacylated suppressor tRNAs. Methods for synthesizing amino acids and aminoacylating tRNA are known in the art. Transcription and translation of plasmids containing nonsense mutations is carried out in a cell free system comprising an E. coli S30 extract and commercially available enzymes and other reagents. Proteins are purified by chromatography. See, for example, Robertson et al., J. Am. Chem. Soc. 113:2722, 1991; Ellman et al., Methods Enzymol. 202:301, 1991; Chung et al., Science 259:806-9, 1993; and Chung et al., Proc. Natl. Acad. Sci. USA 90:10145-9, 1993). In a second method, translation is carried out in Xenopus oocytes by microinjection of mutated mRNA and chemically aminoacylated suppressor tRNAs (Turcatti et al., J. Biol. Chem. 271:19991-8, 1996). Within a third method, E. coli cells are cultured in the absence of a natural amino acid that is to be replaced (e.g., phenylalanine) and in the presence of the desired non-naturally occurring amino acid(s) (e.g., 2-azaphenylalanine, 3-azaphenylalanine, 4-azaphenylalanine, or 4-fluorophenylalanine). The non-naturally occurring amino acid is incorporated into the polypeptide in place of its natural counterpart. See, Koide et al., Biochem. 33:7470-6, 1994. Naturally occurring amino acid residues can be converted to non-naturally occurring species by in vitro chemical modification. Chemical modification can be combined with site-directed mutagenesis to further expand the range of substitutions (Wynn and Richards, Protein Sci. 2:395-403, 1993).

A limited number of non-conservative amino acids, amino acids that are not encoded by the genetic code, non-naturally occurring amino acids, and unnatural amino acids may be substituted for amino acid residues of polypeptides of the present invention.

Essential amino acids in the polypeptides of the present invention can be identified according to procedures known in the art, such as site-directed mutagenesis or alanine-scanning mutagenesis (Cunningham and Wells, Science 244:1081-5, 1989). Sites of biological interaction can also be determined by physical analysis of structure, as determined by such techniques as nuclear magnetic resonance, crystallography, electron diffraction or photoaffinity labeling, in conjunction with mutation of putative contact site amino acids. See, for example, de Vos et al., Science 255:306-12, 1992; Smith et al., J. Mol. Biol. 224:899-904, 1992; Wlodaver et al., FEBS Lett. 309:59-64, 1992. The identities of essential amino acids can also be inferred from analysis of homologies with related components (e.g. the translocation or protease components) of the polypeptides of the present invention.

Multiple amino acid substitutions can be made and tested using known methods of mutagenesis and screening, such as those disclosed by Reidhaar-Olson and Sauer (Science 241:53-7, 1988) or Bowie and Sauer (Proc. Natl. Acad. Sci. USA 86:2152-6, 1989). Briefly, these authors disclose methods for simultaneously randomizing two or more positions in a polypeptide, selecting for functional polypeptide, and then sequencing the mutagenized polypeptides to determine the spectrum of allowable substitutions at each position. Other methods that can be used include phage display (e.g., Lowman et al., Biochem. 30:10832-7, 1991; Ladner et al., U.S. Pat. No. 5,223,409; Huse, WIPO Publication WO 92/06204) and region-directed mutagenesis (Derbyshire et al., Gene 46:145, 1986; Ner et al., DNA 7:127, 1988).

Unless defined otherwise, all technical and scientific terms used herein have the same meaning as commonly understood by one of ordinary skill in the art to which this disclosure belongs. Singleton, et al., DICTIONARY OF MICROBIOLOGY AND MOLECULAR BIOLOGY, ED., John Wiley and Sons, New York (1994), and Hale & Marham, THE HARPER COLLINS DICTIONARY OF BIOLOGY, Harper Perennial, NY (1991) provide the skilled person with a general dictionary of many of the terms used in this disclosure.

This disclosure is not limited by the exemplary methods and materials disclosed herein, and any methods and materials similar or equivalent to those described herein can be used in the practice or testing of embodiments of this disclosure. Numeric ranges are inclusive of the numbers defining the range. Unless otherwise indicated, any nucleic acid sequences are written left to right in 5′ to 3′ orientation; amino acid sequences are written left to right in amino to carboxy orientation, respectively.

The headings provided herein are not limitations of the various aspects or embodiments of this disclosure.

Amino acids are referred to herein using the name of the amino acid, the three letter abbreviation or the single letter abbreviation. The term “protein”, as used herein, includes proteins, polypeptides, and peptides. As used herein, the term “amino acid sequence” is synonymous with the term “polypeptide” and/or the term “protein”. In some instances, the term “amino acid sequence” is synonymous with the term “peptide”. In some instances, the term “amino acid sequence” is synonymous with the term “enzyme”. The terms “protein” and “polypeptide” are used interchangeably herein. In the present disclosure and claims, the conventional one-letter and three-letter codes for amino acid residues may be used. The 3-letter code for amino acids as defined in conformity with the IUPACIUB Joint Commission on Biochemical Nomenclature (JCBN). It is also understood that a polypeptide may be coded for by more than one nucleotide sequence due to the degeneracy of the genetic code.

Other definitions of terms may appear throughout the specification. Before the exemplary embodiments are described in more detail, it is to be understood that this disclosure is not limited to particular embodiments described, and as such may vary. It is also to be understood that the terminology used herein is for the purpose of describing particular embodiments only, and is not intended to be limiting, since the scope of the present disclosure will be defined only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within this disclosure. The upper and lower limits of these smaller ranges may independently be included or excluded in the range, and each range where either, neither or both limits are included in the smaller ranges is also encompassed within this disclosure, subject to any specifically excluded limit in the stated range. Where the stated range includes one or both of the limits, ranges excluding either or both of those included limits are also included in this disclosure.

It must be noted that as used herein and in the appended claims, the singular forms “a”, “an”, and “the” include plural referents unless the context clearly dictates otherwise. Thus, for example, reference to “a modified botulinum neurotoxin A” includes a plurality of such candidate agents and reference to “the modified botulinum neurotoxin A” includes reference to one or more modified botulinum neurotoxin As and equivalents thereof known to those skilled in the art, and so forth.

The publications discussed herein are provided solely for their disclosure prior to the filing date of the present application. Nothing herein is to be construed as an admission that such publications constitute prior art to the claims appended hereto.

### BRIEF DESCRIPTION OF THE DRAWINGS

Embodiments of the invention will now be described, by way of example only, with reference to the following Figures and Examples.

FIG. 1 shows the FDA approved dosages of Dysport® for treating cervical dystonia.

FIG. 2 shows SDS-PAGE of purified recombinant BoNT/AB chimera 1, 2 and 3A (SEQ ID NO: 3, 4 and 5 respectively). Lanes are labelled “Marker” (molecular weight marker), “-DTT” (oxidised BoNT/AB chimera sample), and “+DTT” (reduced BoNT/AB chimera sample).

FIG. 3 shows cleavage of SNAP-25 in rat spinal cord neurones by recombinant BoNT/AB chimera 1, 2 and 3A (SEQ ID NO: 3, 4 and 5 respectively). Cultured rat primary spinal cord neurons (SCN) were exposed to various concentrations of recombinant BoNT/AB chimera 1, 2 or 3A for 24 hours, at 37° C. in a humidified atmosphere with 10% CO₂. Cells were then lysed with 1× NuPAGE buffer supplemented with DTT and Benzonase. The samples were transferred to microcentrifuge tubes, heated for 5 min at 90° C. on heat block and stored at −20° C., before analysis of SNAP-25 cleavage by Western blot. SNAP-25 was detected using a polyclonal antibody, that detects both the full length and cleaved forms of SNAP-25 (Sigma #S9684). Anti-rabbit HRP (Sigma #A6154) was used as the secondary antibody.

FIG. 4 shows mouse digit abduction scoring assay. Mice were injected into the gastrocnemius-soleus complex muscles of one hind limb, under short general anaesthesia; muscle weakening was measured on a 0-4 scale using the digit abduction score (DAS). DAS max values were determined for each dose and plotted against dose and the data were fitted to a 4-parameter logistic equation, ED₅₀ and dose leading to DAS 4 (DAS 4 dose) values were determined.

FIG. 5 shows SDS-PAGE of purified recombinant BoNT/AB chimera 3B and 3C (SEQ ID NO: 6 and 7 respectively). Lanes are labelled “Marker” (molecular weight marker), “-DTT” (oxidised BoNT/AB chimera sample), and “+DTT” (reduced BoNT/AB chimera sample).

FIG. 6 shows cleavage of SNAP-25 by BoNT/A and BoNT/AB chimera 3B and 3C (SEQ ID NO: 2, 6 and 7 respectively) in human induced pluripotent stem cell derived peripheral neurons (PERI.4U—Axiogenesis, Germany). PERI.4U cells were exposed to various concentrations of recombinant BoNT/A, or BoNT/AB chimera 3B or 3C for 24 hours, at 37° C. in a humidified CO₂ atmosphere containing 5% CO₂. Cells were then lysed with 1x NuPAGE buffer supplemented with DTT and Benzonase. The samples were transferred to microcentrifuge tubes, heated for 5 min at 90° C. on heat block and stored at −20° C., before analysis of SNAP-25 cleavage by Western blot. SNAP-25 was detected using a polyclonal antibody, that detects both the full length and cleaved forms of SNAP-25 (Sigma #S9684). Anti-rabbit HRP (Sigma #A6154) was used as the secondary antibody.

FIG. 7 shows duration of muscle weakening over time in the mouse digit abduction scoring assay. Mice were injected into the gastrocnemius-soleus complex muscles of one hind limb, under short general anaesthesia; muscle weakening was measured on a 0-4 scale using the digit abduction score (DAS). Animals of the group injected with the lowest dose that induced during the first four days of injection a DAS of 4 were monitored until complete recovery of the muscle weakness to a DAS of 0 (no observed muscle weakness).

### SEQUENCE LISTING

Where an initial Met amino acid residue or a corresponding initial codon is indicated in any of the following SEQ ID NOs, said residue/codon is optional.

(Nucleotide Sequence of Unmodified BoNT/A)
SEQ ID NO: 1
ATGCCATTCGTCAACAAGCAATTCAACTACAAAGACCCAGTCAACGGCGTCGACATCGCATACAT
CAAGATTCCGAACGCCGGTCAAATGCAGCCGGTTAAGGCTTTTAAGATCCACAACAAGATTTGGG
TTATCCCGGAGCGTGACACCTTCACGAACCCGGAAGAAGGCGATCTGAACCCGCCACCGGAAGCG
AAGCAAGTCCCTGTCAGCTACTACGATTCGACGTACCTGAGCACGGATAACGAAAAAGATAACTA
CCTGAAAGGTGTGACCAAGCTGTTCGAACGTATCTACAGCACGGATCTGGGTCGCATGCTGCTGA
CTAGCATTGTTCGCGGTATCCCGTTCTGGGGTGGTAGCACGATTGACACCGAACTGAAGGTTATC
GACACTAACTGCATTAACGTTATTCAACCGGATGGTAGCTATCGTAGCGAAGAGCTGAATCTGGT
CATCATTGGCCCGAGCGCAGACATTATCCAATTCGAGTGCAAGAGCTTTGGTCACGAGGTTCTGA
ATCTGACCCGCAATGGCTATGGTAGCACCCAGTACATTCGTTTTTCGCCGGATTTTACCTTCGGC
TTTGAAGAGAGCCTGGAGGTTGATACCAATCCGTTGCTGGGTGCGGGCAAATTCGCTACCGATCC
GGCTGTCACGCTGGCCCATGAACTGATCCACGCAGGCCACCGCCTGTACGGCATTGCCATCAACC
CAAACCGTGTGTTCAAGGTTAATACGAATGCATACTACGAGATGAGCGGCCTGGAAGTCAGCTTC
GAAGAACTGCGCACCTTCGGTGGCCATGACGCTAAATTCATTGACAGCTTGCAAGAGAATGAGTT
CCGTCTGTACTACTATAACAAATTCAAAGACATTGCAAGCACGTTGAACAAGGCCAAAAGCATCG
TTGGTACTACCGCGTCGTTGCAGTATATGAAGAATGTGTTTAAAGAGAAGTACCTGCTGTCCGAG
GATACCTCCGGCAAGTTTAGCGTTGATAAGCTGAAGTTTGACAAACTGTACAAGATGCTGACCGA
GATTTACACCGAGGACAACTTTGTGAAATTCTTCAAAGTGTTGAATCGTAAAACCTATCTGAATT
TTGACAAAGCGGTTTTCAAGATTAACATCGTGCCGAAGGTGAACTACACCATCTATGACGGTTTT
AACCTGCGTAACACCAACCTGGCGGCGAACTTTAACGGTCAGAATACGGAAATCAACAACATGAA
TTTCACGAAGTTGAAGAACTTCACGGGTCTGTTCGAGTTCTATAAGCTGCTGTGCGTGCGCGGTA
TCATCACCAGCAAAACCAAAAGCCTGGACAAAGGCTACAACAAGGCGCTGAATGACCTGTGCATT
AAGGTAAACAATTGGGATCTGTTCTTTTCGCCATCCGAAGATAATTTTACCAACGACCTGAACAA
GGGTGAAGAAATCACCAGCGATACGAATATTGAAGCAGCGGAAGAGAATATCAGCCTGGATCTGA
TCCAGCAGTACTATCTGACCTTTAACTTCGACAATGAACCGGAGAACATTAGCATTGAGAATCTG
AGCAGCGACATTATCGGTCAGCTGGAACTGATGCCGAATATCGAACGTTTCCCGAACGGCAAAAA
GTACGAGCTGGACAAGTACACTATGTTCCATTACCTGCGTGCACAGGAGTTTGAACACGGTAAAA
GCCGTATCGCGCTGACCAACAGCGTTAACGAGGCCCTGCTGAACCCGAGCCGTGTCTATACCTTC
TTCAGCAGCGACTATGTTAAGAAAGTGAACAAAGCCACTGAGGCCGCGATGTTCCTGGGCTGGGT
GGAACAGCTGGTATATGACTTCACGGACGAGACGAGCGAAGTGAGCACTACCGACAAAATTGCTG
ATATTACCATCATTATCCCGTATATTGGTCCGGCACTGAACATTGGCAACATGCTGTACAAAGAC
GATTTTGTGGGTGCCCTGATCTTCTCCGGTGCCGTGATTCTGCTGGAGTTCATTCCGGAGATTGC
GATCCCGGTGTTGGGTACCTTCGCGCTGGTGTCCTACATCGCGAATAAGGTTCTGACGGTTCAGA
CCATCGATAACGCGCTGTCGAAACGTAATGAAAAATGGGACGAGGTTTACAAATACATTGTTACG
AATTGGCTGGCGAAAGTCAATACCCAGATCGACCTGATCCGTAAGAAAATGAAAGAGGCGCTGGA
GAATCAGGCGGAGGCCACCAAAGCAATTATCAACTACCAATACAACCAGTACACGGAAGAAGAGA
AGAATAACATTAACTTCAATATCGATGATTTGAGCAGCAAGCTGAATGAATCTATCAACAAAGCG
ATGATCAATATCAACAAGTTTTTGAATCAGTGTAGCGTTTCGTACCTGATGAATAGCATGATTCC
GTATGGCGTCAAACGTCTGGAGGACTTCGACGCCAGCCTGAAAGATGCGTTGCTGAAATACATTT
ACGACAATCGTGGTACGCTGATTGGCCAAGTTGACCGCTTGAAAGACAAAGTTAACAATACCCTG
AGCACCGACATCCCATTTCAACTGAGCAAGTATGTTGATAATCAACGTCTGTTGAGCACTTTCAC
CGAGTATATCAAAAACATCATCAATACTAGCATTCTGAACCTGCGTTACGAGAGCAATCATCTGA
TTGATCTGAGCCGTTATGCAAGCAAGATCAACATCGGTAGCAAGGTCAATTTTGACCCGATCGAT
AAGAACCAGATCCAGCTGTTTAATCTGGAATCGAGCAAAATTGAGGTTATCCTGAAAAACGCCAT
TGTCTACAACTCCATGTACGAGAATTTCTCCACCAGCTTCTGGATTCGCATCCCGAAATACTTCA
ACAGCATTAGCCTGAACAACGAGTATACTATCATCAACTGTATGGAGAACAACAGCGGTTGGAAG
GTGTCTCTGAACTATGGTGAGATCATTTGGACCTTGCAGGACACCCAAGAGATCAAGCAGCGCGT
CGTGTTCAAGTACTCTCAAATGATCAACATTTCCGATTACATTAATCGTTGGATCTTCGTGACCA
TTACGAATAACCGTCTGAATAACAGCAAGATTTACATCAATGGTCGCTTGATCGATCAGAAACCG
ATTAGCAACCTGGGTAATATCCACGCAAGCAACAACATTATGTTCAAATTGGACGGTTGCCGCGA
TACCCATCGTTATATCTGGATCAAGTATTTCAACCTGTTTGATAAAGAACTGAATGAGAAGGAGA
TCAAAGATTTGTATGACAACCAATCTAACAGCGGCATTTTGAAGGACTTCTGGGGCGATTATCTG
CAATACGATAAGCCGTACTATATGCTGAACCTGTATGATCCGAACAAATATGTGGATGTCAATAA
TGTGGGTATTCGTGGTTACATGTATTTGAAGGGTCCGCGTGGCAGCGTTATGACGACCAACATTT
ACCTGAACTCTAGCCTGTACCGTGGTACGAAATTCATCATTAAGAAATATGCCAGCGGCAACAAA
GATAACATTGTGCGTAATAACGATCGTGTCTACATCAACGTGGTCGTGAAGAATAAAGAGTACCG
TCTGGCGACCAACGCTTCGCAGGCGGGTGTTGAGAAAATTCTGAGCGCGTTGGAGATCCCTGATG
TCGGTAATCTGAGCCAAGTCGTGGTTATGAAGAGCAAGAACGACCAGGGTATCACTAACAAGTGC
AAGATGAACCTGCAAGACAACAATGGTAACGACATCGGCTTTATTGGTTTCCACCAGTTCAACAA
TATTGCTAAACTGGTAGCGAGCAATTGGTACAATCGTCAGATTGAGCGCAGCAGCCGTACTTTGG
GCTGTAGCTGGGAGTTTATCCCGGTCGATGATGGTTGGGGCGAACGTCCGCTG
(Polypeptide Sequence of Unmodified BoNT/A)
SEQ ID NO: 2
MPFVNKQFNYKDPVNGVDIAYIKIPNAGQMQPVKAFKIHNKIWVIPERDTFTNPEEGDLNPPPEA
KQVPVSYYDSTYLSTDNEKDNYLKGVTKLFERIYSTDLGRMLLTSIVRGIPFWGGSTIDTELKVI
DTNCINVIQPDGSYRSEELNLVIIGPSADIIQFECKSFGHEVLNLTRNGYGSTQYIRFSPDFTFG
FEESLEVDTNPLLGAGKFATDPAVTLAHELIHAGHRLYGIAINPNRVFKVNTNAYYEMSGLEVSF
EELRTFGGHDAKFIDSLQENEFRLYYYNKFKDIASTLNKAKSIVGTTASLQYMKNVFKEKYLLSE
DTSGKFSVDKLKFDKLYKMLTEIYTEDNFVKFFKVLNRKTYLNFDKAVFKINIVPKVNYTIYDGF
NLRNTNLAANFNGQNTEINNMNFTKLKNFTGLFEFYKLLCVRGIITSKTKSLDKGYNKALNDLCI
KVNNWDLFFSPSEDNFTNDLNKGEEITSDTNIEAAEENISLDLIQQYYLTFNFDNEPENISIENL
SSDIIGQLELMPNIERFPNGKKYELDKYTMFHYLRAQEFEHGKSRIALTNSVNEALLNPSRVYTF
FSSDYVKKVNKATEAAMFLGWVEQLVYDFTDETSEVSTTDKIADITIIIPYIGPALNIGNMLYKD
DFVGALIFSGAVILLEFIPEIAIPVLGTFALVSYIANKVLTVQTIDNALSKRNEKWDEVYKYIVT
NWLAKVNTQIDLIRKKMKEALENQAEATKAIINYQYNQYTEEEKNNINFNIDDLSSKLNESINKA
MININKFLNQCSVSYLMNSMIPYGVKRLEDFDASLKDALLKYIYDNRGTLIGQVDRLKDKVNNTL
STDIPFQLSKYVDNQRLLSTFTEYIKNIINTSILNLRYESNHLIDLSRYASKINIGSKVNFDPID
KNQIQLENLESSKIEVILKNAIVYNSMYENFSTSFWIRIPKYENSISLNNEYTIINCMENNSGWK
VSLNYGEIIWTLQDTQEIKQRVVFKYSQMINISDYINRWIFVTITNNRLNNSKIYINGRLIDQKP
ISNLGNIHASNNIMFKLDGCRDTHRYIWIKYFNLFDKELNEKEIKDLYDNQSNSGILKDFWGDYL
QYDKPYYMLNLYDPNKYVDVNNVGIRGYMYLKGPRGSVMTTNIYLNSSLYRGTKFIIKKYASGNK
DNIVRNNDRVYINVVVKNKEYRLATNASQAGVEKILSALEIPDVGNLSQVVVMKSKNDQGITNKC
KMNLQDNNGNDIGFIGFHQFNNIAKLVASNWYNRQIERSSRTLGCSWEFIPVDDGWGERPL
(Polypeptide Sequence of Modified BoNT/A “Chimera 1”)
SEQ ID NO: 3
MPFVNKQFNYKDPVNGVDIAYIKIPNAGQMQPVKAFKIHNKIWVIPERDTFTNPEEGDLNPPPEA
KQVPVSYYDSTYLSTDNEKDNYLKGVTKLFERIYSTDLGRMLLTSIVRGIPFWGGSTIDTELKVI
DTNCINVIQPDGSYRSEELNLVIIGPSADIIQFECKSFGHEVLNLTRNGYGSTQYIRFSPDFTFG
FEESLEVDTNPLLGAGKFATDPAVTLAHELIHAGHRLYGIAINPNRVFKVNTNAYYEMSGLEVSF
EELRTFGGHDAKFIDSLQENEFRLYYYNKFKDIASTLNKAKSIVGTTASLQYMKNVFKEKYLLSE
DTSGKFSVDKLKEDKLYKMLTEIYTEDNFVKFFKVLNRKTYLNFDKAVFKINIVPKVNYTIYDGE
NLRNTNLAANENGQNTEINNMNFTKLKNFTGLFEFYKLLCVRGIITSKTKSLDKGYNKALNDLCI
KVNNWDLFFSPSEDNETNDLNKGEEITSDTNIEAAEENISLDLIQQYYLTENEDNEPENISIENL
SSDIIGQLELMPNIEREPNGKKYELDKYTMFHYLRAQEFEHGKSRIALTNSVNEALLNPSRVYTF
FSSDYVKKVNKATEAAMFLGWVEQLVYDFTDETSEVSTTDKIADITIIIPYIGPALNIGNMLYKD
DFVGALIFSGAVILLEFIPEIAIPVLGTFALVSYIANKVLTVQTIDNALSKRNEKWDEVYKYIVT
NWLAKVNTQIDLIRKKMKEALENQAEATKAIINYQYNQYTEEEKNNINFNIDDLSSKLNESINKA
MININKELNQCSVSYLMNSMIPYGVKRLEDEDASLKDALLKYIYDNRGTLIGQVDRLKDKVNNTL
STDIPFQLSKYVDNQRLLSTFTEYIKSEILNNIILNLRYKDNNLIDLSGYGAKVEVYDGVELNDK
NQFKLTSSANSKIRVTQNQNIIFNSVELDESVSEWIRIPKYKNDGIQNYIHNEYTIINCMKNNSG
WKISIRGNRIIWTLIDINGKTKSVFFEYNIREDISEYINRWFFVTITNNLNNAKIYINGKLESNT
DIKDIREVIANGEIIFKLDGDIDRTQFIWMKYESIENTELSQSNIEERYKIQSYSEYLKDEWGNP
LMYNKEYYMFNAGNKNSYIKLKKDSPVGEILTRSKYNQNSKYINYRDLYIGEKFIIRRKSNSQSI
NDDIVRKEDYIYLDFFNLNQEWRVYTYKYFKKEEMKLFLAPIYDSDEFYNTIQIKEYDEQPTYSC
QLLFKKDEESTDEIGLIGIHRFYESGIVFEEYKDYFCISKWYLKEVKRKPYNLKLGCNWQFIPKD
EGWTEHHHHHHHHHH
(Polypeptide Sequence of Modified BoNT/A “Chimera 2”)
SEQ ID NO: 4
MPFVNKQFNYKDPVNGVDIAYIKIPNAGQMQPVKAFKIHNKIWVIPERDTFTNPEEGDLNPPPEA
KQVPVSYYDSTYLSTDNEKDNYLKGVTKLFERIYSTDLGRMLLTSIVRGIPFWGGSTIDTELKVI
DTNCINVIQPDGSYRSEELNLVIIGPSADIIQFECKSFGHEVLNLTRNGYGSTQYIRFSPDFTFG
FEESLEVDTNPLLGAGKFATDPAVTLAHELIHAGHRLYGIAINPNRVFKVNTNAYYEMSGLEVSF
EELRTFGGHDAKFIDSLQENEFRLYYYNKFKDIASTLNKAKSIVGTTASLQYMKNVFKEKYLLSE
DTSGKFSVDKLKEDKLYKMLTEIYTEDNFVKFFKVLNRKTYLNFDKAVFKINIVPKVNYTIYDGE
NLRNTNLAANENGQNTEINNMNFTKLKNFTGLFEFYKLLCVRGIITSKTKSLDKGYNKALNDLCI
KVNNWDLFFSPSEDNFTNDLNKGEEITSDTNIEAAEENISLDLIQQYYLTFNFDNEPENISIENL
SSDIIGQLELMPNIEREPNGKKYELDKYTMFHYLRAQEFEHGKSRIALTNSVNEALLNPSRVYTF
FSSDYVKKVNKATEAAMFLGWVEQLVYDFTDETSEVSTTDKIADITIIIPYIGPALNIGNMLYKD
DEVGALIFSGAVILLEFIPEIAIPVLGTFALVSYIANKVLTVQTIDNALSKRNEKWDEVYKYIVT
NWLAKVNTQIDLIRKKMKEALENQAEATKAIINYQYNQYTEEEKNNINFNIDDLSSKLNESINKA
MININKFLNQCSVSYLMNSMIPYGVKRLEDEDASLKDALLKYIYDNRGTLIGQVDRLKDKVNNTL
STDIPFQLSKYVDNQRLLSTFTEYIKNIIELGGGGSELSEILNNIILNLRYKDNNLIDLSGYGAK
VEVYDGVELNDKNQFKLTSSANSKIRVTQNQNIIFNSVELDESVSEWIRIPKYKNDGIQNYIHNE
YTIINCMKNNSGWKISIRGNRIIWTLIDINGKTKSVFFEYNIREDISEYINRWFFVTITNNLNNA
KIYINGKLESNTDIKDIREVIANGEIIFKLDGDIDRTQFIWMKYFSIFNTELSQSNIEERYKIQS
YSEYLKDEWGNPLMYNKEYYMENAGNKNSYIKLKKDSPVGEILTRSKYNQNSKYINYRDLYIGEK
FIIRRKSNSQSINDDIVRKEDYIYLDFENLNQEWRVYTYKYFKKEEMKLFLAPIYDSDEFYNTIQ
IKEYDEQPTYSCQLLFKKDEESTDEIGLIGIHRFYESGIVFEEYKDYFCISKWYLKEVKRKPYNL
KLGCNWQFIPKDEGWTEHHHHHHHHHH
(Polypeptide Sequence of Modified BoNT/A “Chimera 3A”)
SEQ ID NO: 5
MPFVNKQFNYKDPVNGVDIAYIKIPNAGQMQPVKAFKIHNKIWVIPERDTFTNPEEGDLNPPPEA
KQVPVSYYDSTYLSTDNEKDNYLKGVTKLFERIYSTDLGRMLLTSIVRGIPFWGGSTIDTELKVI
DTNCINVIQPDGSYRSEELNLVIIGPSADIIQFECKSFGHEVLNLTRNGYGSTQYIRFSPDFTFG
FEESLEVDTNPLLGAGKFATDPAVTLAHELIHAGHRLYGIAINPNRVFKVNTNAYYEMSGLEVSF
EELRTFGGHDAKFIDSLQENEFRLYYYNKFKDIASTLNKAKSIVGTTASLQYMKNVFKEKYLLSE
DTSGKFSVDKLKEDKLYKMLTEIYTEDNFVKFFKVLNRKTYLNFDKAVFKINIVPKVNYTIYDGE
NLRNTNLAANENGQNTEINNMNFTKLKNFTGLFEFYKLLCVRGIITSKTKSLDKGYNKALNDLCI
KVNNWDLFFSPSEDNFTNDLNKGEEITSDTNIEAAEENISLDLIQQYYLTENEDNEPENISIENL
SSDIIGQLELMPNIEREPNGKKYELDKYTMFHYLRAQEFEHGKSRIALTNSVNEALLNPSRVYTF
FSSDYVKKVNKATEAAMFLGWVEQLVYDFTDETSEVSTTDKIADITIIIPYIGPALNIGNMLYKD
DFVGALIFSGAVILLEFIPEIAIPVLGTFALVSYIANKVLTVQTIDNALSKRNEKWDEVYKYIVT
NWLAKVNTQIDLIRKKMKEALENQAEATKAIINYQYNQYTEEEKNNINFNIDDLSSKLNESINKA
MININKFLNQCSVSYLMNSMIPYGVKRLEDFDASLKDALLKYIYDNRGTLIGQVDRLKDKVNNTL
STDIPFQLSKYVDNQRLLSTFTEYIKNILNNIILNLRYKDNNLIDLSGYGAKVEVYDGVELNDKN
QFKLTSSANSKIRVTQNQNIIENSVELDESVSEWIRIPKYKNDGIQNYIHNEYTIINCMKNNSGW
KISIRGNRIIWTLIDINGKTKSVFFEYNIREDISEYINRWFFVTITNNLNNAKIYINGKLESNTD
IKDIREVIANGEIIFKLDGDIDRTQFIWMKYFSIFNTELSQSNIEERYKIQSYSEYLKDFWGNPL
MYNKEYYMENAGNKNSYIKLKKDSPVGEILTRSKYNQNSKYINYRDLYIGEKFIIRRKSNSQSIN
DDIVRKEDYIYLDFFNLNQEWRVYTYKYFKKEEMKLFLAPIYDSDEFYNTIQIKEYDEQPTYSCQ
LLFKKDEESTDEIGLIGIHRFYESGIVFEEYKDYFCISKWYLKEVKRKPYNLKLGCNWQFIPKDE
GWTEHHHHHHHHHH
(Polypeptide Sequence of Modified BoNT/A “Chimera 3B”)
SEQ ID NO: 6
MPFVNKQFNYKDPVNGVDIAYIKIPNAGQMQPVKAFKIHNKIWVIPERDTFTNPEEGDLNPPPEA
KQVPVSYYDSTYLSTDNEKDNYLKGVTKLFERIYSTDLGRMLLTSIVRGIPFWGGSTIDTELKVI
DTNCINVIQPDGSYRSEELNLVIIGPSADIIQFECKSFGHEVLNLTRNGYGSTQYIRFSPDFTFG
FEESLEVDTNPLLGAGKFATDPAVTLAHELIHAGHRLYGIAINPNRVFKVNTNAYYEMSGLEVSF
EELRTFGGHDAKFIDSLQENEFRLYYYNKFKDIASTLNKAKSIVGTTASLQYMKNVFKEKYLLSE
DTSGKFSVDKLKFDKLYKMLTEIYTEDNFVKFFKVLNRKTYLNFDKAVEKINIVPKVNYTIYDGF
NLRNTNLAANFNGQNTEINNMNFTKLKNFTGLFEFYKLLCVRGIITSKTKSLDKGYNKALNDLCI
KVNNWDLFFSPSEDNFTNDLNKGEEITSDTNIEAAEENISLDLIQQYYLTFNFDNEPENISIENL
SSDIIGQLELMPNIERFPNGKKYELDKYTMFHYLRAQEFEHGKSRIALTNSVNEALLNPSRVYTF
FSSDYVKKVNKATEAAMFLGWVEQLVYDFTDETSEVSTTDKIADITIIIPYIGPALNIGNMLYKD
DFVGALIFSGAVILLEFIPEIAIPVLGTFALVSYIANKVLTVQTIDNALSKRNEKWDEVYKYIVT
NWLAKVNTQIDLIRKKMKEALENQAEATKAIINYQYNQYTEEEKNNINFNIDDLSSKLNESINKA
MININKFLNQCSVSYLMNSMIPYGVKRLEDFDASLKDALLKYIYDNRGTLIGQVDRLKDKVNNTL
STDIPFQLSKYVDNQRLLSTFTEYIKNILNNIILNLRYKDNNLIDLSGYGAKVEVYDGVELNDKN
QFKLTSSANSKIRVTQNQNIIFNSVFLDFSVSFWIRIPKYKNDGIQNYIHNEYTIINCMKNNSGW
KISIRGNRIIWTLIDINGKTKSVFFEYNIREDISEYINRWFFVTITNNLNNAKIYINGKLESNTD
IKDIREVIANGEIIFKLDGDIDRTQFIWMKYFSIFNTELSQSNIEERYKIQSYSEYLKDFWGNPL
MYNKEYYMFNAGNKNSYIKLKKDSPVGEILTRSKYNQNSKYINYRDLYIGEKFIIRRKSNSQSIN
DDIVRKEDYIYLDFFNLNQEWRVYTYKYFKKEEMKLFLAPIYDSDEFYNTIQIKEYDEQPTYSCQ
LLFKKDEESTDEIGLIGIHRFYESGIVFEEYKDYFCISKWYLKEVKRKPYNLKLGCNWQFIPKDE
GWTE
(Polypeptide Sequence of Modified BoNT/A “Chimera 3C”)
SEQ ID NO: 7
MPFVNKQFNYKDPVNGVDIAYIKIPNAGQMQPVKAFKIHNKIWVIPERDTFTNPEEGDLNPPPEA
KQVPVSYYDSTYLSTDNEKDNYLKGVTKLFERIYSTDLGRMLLTSIVRGIPFWGGSTIDTELKVI
DTNCINVIQPDGSYRSEELNLVIIGPSADIIQFECKSFGHEVLNLTRNGYGSTQYIRFSPDFTFG
FEESLEVDTNPLLGAGKFATDPAVTLAHELIHAGHRLYGIAINPNRVFKVNTNAYYEMSGLEVSF
EELRTFGGHDAKFIDSLQENEFRLYYYNKFKDIASTLNKAKSIVGTTASLQYMKNVFKEKYLLSE
DTSGKFSVDKLKFDKLYKMLTEIYTEDNFVKFFKVLNRKTYLNFDKAVFKINIVPKVNYTIYDGF
NLRNTNLAANFNGQNTEINNMNFTKLKNFTGLFEFYKLLCVRGIITSKTKSLDKGYNKALNDLCI
KVNNWDLFFSPSEDNFTNDLNKGEEITSDTNIEAAEENISLDLIQQYYLTFNFDNEPENISIENL
SSDIIGQLELMPNIERFPNGKKYELDKYTMFHYLRAQEFEHGKSRIALTNSVNEALLNPSRVYTF
FSSDYVKKVNKATEAAMFLGWVEQLVYDFTDETSEVSTTDKIADITIIIPYIGPALNIGNMLYKD
DFVGALIFSGAVILLEFIPEIAIPVLGTFALVSYIANKVLTVQTIDNALSKRNEKWDEVYKYIVT
NWLAKVNTQIDLIRKKMKEALENQAEATKAIINYQYNQYTEEEKNNINFNIDDLSSKLNESINKA
MININKFLNQCSVSYLMNSMIPYGVKRLEDFDASLKDALLKYIYDNRGTLIGQVDRLKDKVNNTL
STDIPFQLSKYVDNQRLLSTFTEYIKNILNNIILNLRYKDNNLIDLSGYGAKVEVYDGVELNDKN
QFKLTSSANSKIRVTQNQNIIFNSVFLDFSVSFWIRIPKYKNDGIQNYIHNEYTIINCMKNNSGW
KISIRGNRIIWTLIDINGKTKSVFFEYNIREDISEYINRWFFVTITNNLNNAKIYINGKLESNTD
IKDIREVIANGEIIFKLDGDIDRTQFIWMKYFSIFNTELSQSNIEERYKIQSYSEYLKDFWGNPL
MYNKEYYMFNAGNKNSYIKLKKDSPVGEILTRSKYNQNSKYINYRDLYIGEKFIIRRKSNSQSIN
DDIVRKEDYIYLDFFNLNQEWRVYTYKYFKKEEEKLFLAPISDSDEFYNTIQIKEYDEQPTYSCQ
LLFKKDEESTDEIGLIGIHRFYESGIVFEEYKDYFCISKWYLKEVKRKPYNLKLGCNWQFIPKDE
GWTE
(Polypeptide Sequence of BoNT/B)
SEQ ID NO: 8
MPVTINNENYNDPIDNNNIIMMEPPFARGTGRYYKAFKITDRIWIIPERYTFGYKPEDENKSSGI
FNRDVCEYYDPDYLNTNDKKNIFLQTMIKLENRIKSKPLGEKLLEMIINGIPYLGDRRVPLEEFN
TNIASVTVNKLISNPGEVERKKGIFANLIIFGPGPVLNENETIDIGIQNHFASREGEGGIMQMKF
CPEYVSVENNVQENKGASIENRRGYFSDPALILMHELIHVLHGLYGIKVDDLPIVPNEKKFFMQS
TDAIQAEELYTFGGQDPSIITPSTDKSIYDKVLQNERGIVDRLNKVLVCISDPNININIYKNKFK
DKYKFVEDSEGKYSIDVESFDKLYKSLMFGFTETNIAENYKIKTRASYFSDSLPPVKIKNLLDNE
IYTIEEGFNISDKDMEKEYRGQNKAINKQAYEEISKEHLAVYKIQMCKSVKAPGICIDVDNEDLF
FIADKNSESDDLSKNERIEYNTQSNYIENDFPINELILDTDLISKIELPSENTESLTDFNVDVPV
YEKQPAIKKIFTDENTIFQYLYSQTEPLDIRDISLTSSEDDALLFSNKVYSFFSMDYIKTANKVV
EAGLFAGWVKQIVNDFVIEANKSNTMDKIADISLIVPYIGLALNVGNETAKGNFENAFEIAGASI
LLEFIPELLIPVVGAFLLESYIDNKNKIIKTIDNALTKRNEKWSDMYGLIVAQWLSTVNTQFYTI
KEGMYKALNYQAQALEEIIKYRYNIYSEKEKSNINIDENDINSKLNEGINQAIDNINNFINGCSV
SYLMKKMIPLAVEKLLDFDNTLKKNLLNYIDENKLYLIGSAEYEKSKVNKYLKTIMPEDLSIYTN
DTILIEMENKYNSEILNNIILNLRYKDNNLIDLSGYGAKVEVYDGVELNDKNQFKLTSSANSKIR
VTQNQNIIFNSVFLDESVSFWIRIPKYKNDGIQNYIHNEYTIINCMKNNSGWKISIRGNRIIWTL
IDINGKTKSVFFEYNIREDISEYINRWFFVTITNNLNNAKIYINGKLESNTDIKDIREVIANGEI
IFKLDGDIDRTQFIWMKYFSIENTELSQSNIEERYKIQSYSEYLKDFWGNPLMYNKEYYMENAGN
KNSYIKLKKDSPVGEILTRSKYNQNSKYINYRDLYIGEKFIIRRKSNSQSINDDIVRKEDYIYLD
FENLNQEWRVYTYKYFKKEEEKLFLAPISDSDEFYNTIQIKEYDEQPTYSCQLLFKKDEESTDEI
GLIGIHRFYESGIVFEEYKDYFCISKWYLKEVKRKPYNLKLGCNWQFIPKDEGWTE
C-terminal L-chain Fragment
SEQ ID NO: 9
TKSLDKGYNK
C-terminal L-chain Fragment 2
SEQ ID NO: 10
SLDKGYNK
Di-Chain L-Chain 1
SEQ ID NO: 11
PFVNKQFNYKDPVNGVDIAYIKIPNAGQMQPVKAFKIHNKIWVIPERDTFTNPEEGDLNPPPEAK
QVPVSYYDSTYLSTDNEKDNYLKGVTKLFERIYSTDLGRMLLTSIVRGIPFWGGSTIDTELKVID
TNCINVIQPDGSYRSEELNLVIIGPSADIIQFECKSFGHEVLNLTRNGYGSTQYIRFSPDFTFGF
EESLEVDTNPLLGAGKFATDPAVTLAHELIHAGHRLYGIAINPNRVFKVNTNAYYEMSGLEVSFE
ELRTFGGHDAKFIDSLQENEFRLYYYNKFKDIASTLNKAKSIVGTTASLQYMKNVFKEKYLLSED
TSGKFSVDKLKFDKLYKMLTEIYTEDNFVKFFKVLNRKTYLNEDKAVEKINIVPKVNYTIYDGFN
LRNTNLAANFNGQNTEINNMNFTKLKNFTGLFEFYKLLCVRGIITSK
Di-Chain L-Chain 2
SEQ ID NO: 12
PFVNKQFNYKDPVNGVDIAYIKIPNAGQMQPVKAFKIHNKIWVIPERDTFTNPEEGDLNPPPEAK
QVPVSYYDSTYLSTDNEKDNYLKGVTKLFERIYSTDLGRMLLTSIVRGIPFWGGSTIDTELKVID
TNCINVIQPDGSYRSEELNLVIIGPSADIIQFECKSFGHEVLNLTRNGYGSTQYIRFSPDFTFGF
EESLEVDTNPLLGAGKFATDPAVTLAHELIHAGHRLYGIAINPNRVFKVNTNAYYEMSGLEVSFE
ELRTFGGHDAKFIDSLQENEFRLYYYNKFKDIASTLNKAKSIVGTTASLQYMKNVFKEKYLLSED
TSGKFSVDKLKFDKLYKMLTEIYTEDNFVKFFKVLNRKTYLNFDKAVFKINIVPKVNYTIYDGFN
LRNTNLAANFNGQNTEINNMNFTKLKNFTGLFEFYKLLCVRGIITSKTK
Di-Chain H-Chain
SEQ ID NO: 13
ALNDLCIKVNNWDLFFSPSEDNFTNDLNKGEEITSDTNIEAAEENISLDLIQQYYLTFNFDNEPE
NISIENLSSDIIGQLELMPNIERFPNGKKYELDKYTMFHYLRAQEFEHGKSRIALTNSVNEALLN
PSRVYTFFSSDYVKKVNKATEAAMFLGWVEQLVYDFTDETSEVSTTDKIADITIIIPYIGPALNI
GNMLYKDDFVGALIFSGAVILLEFIPEIAIPVLGTFALVSYIANKVLTVQTIDNALSKRNEKWDE
VYKYIVTNWLAKVNTQIDLIRKKMKEALENQAEATKAIINYQYNQYTEEEKNNINFNIDDLSSKL
NESINKAMININKFLNQCSVSYLMNSMIPYGVKRLEDFDASLKDALLKYIYDNRGTLIGQVDRLK
DKVNNTLSTDIPFQLSKYVDNQRLLSTFTEYIKNILNNIILNLRYKDNNLIDLSGYGAKVEVYDG
VELNDKNQFKLTSSANSKIRVTQNQNIIFNSVFLDFSVSFWIRIPKYKNDGIQNYIHNEYTIINC
MKNNSGWKISIRGNRIIWTLIDINGKTKSVFFEYNIREDISEYINRWFFVTITNNLNNAKIYING
KLESNTDIKDIREVIANGEIIFKLDGDIDRTQFIWMKYFSIFNTELSQSNIEERYKIQSYSEYLK
DFWGNPLMYNKEYYMFNAGNKNSYIKLKKDSPVGEILTRSKYNQNSKYINYRDLYIGEKFIIRRK
SNSQSINDDIVRKEDYIYLDFFNLNQEWRVYTYKYFKKEEMKLFLAPIYDSDEFYNTIQIKEYDE
QPTYSCQLLFKKDEESTDEIGLIGIHRFYESGIVFEEYKDYFCISKWYLKEVKRKPYNLKLGCNW
QFIPKDEGWTE

### EXAMPLES

### Example 1

### Cloning, Expression and Purification of Modified BoNT/a (BoNT/AB Chimeras)

BONT/AB chimeric constructs 1, 2, 3A, 3B, and 3C (SEQ ID NO: 3 to 7, respectively) were constructed from DNA encoding the parent serotype molecule and appropriate oligonucleotides using standard molecular biology techniques. These were then cloned into the pJ401 expression vector with or without a C-terminal His₁₀-tag and transformed into BLR (DE3) E. coli cells for over-expression. These cells were grown at 37° C. and 225 RPM shaking in 2 L baffled conical flasks containing 1 L modified Terrific Broth (mTB) supplemented with the appropriate antibiotic. Once the A₆₀₀ reached >0.5, the incubator temperature was decreased to 16° C., and then induced with 1 mM IPTG an hour later for 20 h at 225 RPM shaking, to express the recombinant BoNT/AB construct.

Harvested cells were lysed by ultrasonication and clarified by centrifugation at 4500 RPM for 1 h at 4° C. The recombinant BoNT/AB chimeric molecules were then extracted in ammonium sulphate and purified by standard fast protein liquid chromatography (FPLC) techniques. This involved using a hydrophobic interaction resin for capture and an anion-exchange resin for the intermediate purification step. The partially purified molecules were then proteolytically cleaved with endoproteinase Lys-C to yield the active di-chain. This was further purified with a second hydrophobic interaction resin to obtain the final BoNT/AB chimera.

For BoNT/AB chimeric molecules with a decahistadine tag (H₁₀) (chimera 1, 2, 3A), the capture step employed the use of an immobilised nickel resin instead of the hydrophobic interaction resin.

The sequence of each chimera is presented in Table 1.

TABLE 1
chimeric BoNT/AB constructs

| | SEQ | |
|---|---|---|
| Molecule | ID NO | Sequence |
| Chimera 1 | 3 | A1: 1-871 + B1: 858-1291 (E1191M/S1199Y) + |
| | | His₁₀-tag |
| Chimera 2 | 4 | A1: 1-874 + ELGGGGSEL + B1: 858-1291 |
| | | (E1191M/S1199Y) + His₁₀-tag |
| Chimera 3A | 5 | A1: 1-872 + B1: 860-1291 (E1191M/S1199Y) + |
| | | His₁₀-tag |
| Chimera 3B | 6 | A1: 1-872 + B1: 860-1291 (E1191M/S1199Y) |
| Chimera 3C | 7 | A1: 1-872 + B1: 860-1291 |

### Example 2

### Comparison of BoNT/AB Chimera 1, 2 and 3A

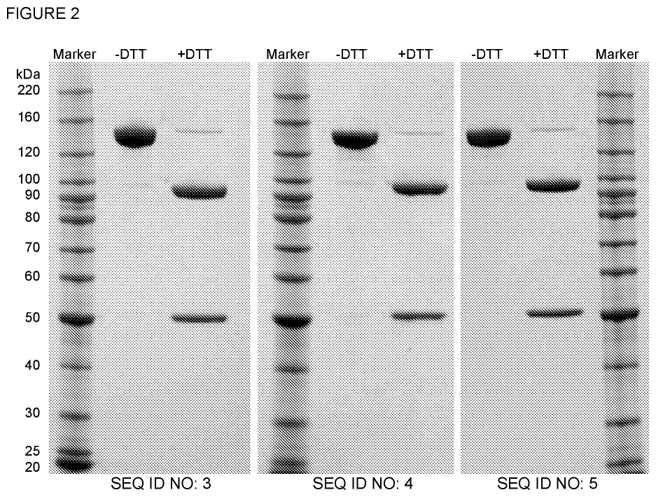

BONT/AB chimera 1, 2 and 3A which have a C-terminal His₁₀ tag and E1191M/S1199Y double mutation were purified as described in Example 1 (FIG. 2) and tested for functional activity.

### Rat Spinal Cord Neurons Snap-25 Cleavage Assay

Primary cultures of rat spinal cord neurons (SCN) were prepared and grown, for 3 weeks, in 96 well tissue culture plates (as described in: Masuyer et al., 2011, J. Struct. Biol. Structure and activity of a functional derivative of Clostridium botulinum neurotoxin B; and in: Chaddock et al., 2002, Protein Expr. Purif. Expression and purification of catalytically active, non-toxic endopeptidase derivatives of Clostridium botulinum toxin type A). Serial dilutions of BoNT/AB were prepared in SCN feeding medium. The growth medium from the wells to be treated was collected and filtered (0.2 μm filter). 125 μL of the filtered medium was added back to each test well. 125 μL of diluted toxin was then added to the plate (triplicate wells). The treated cells were incubated at 37° C., 10% CO₂, for 24+1 h).

### Analysis of BONT Activity Using the SNAP-25 Cleavage Assay

Following treatment, BoNT was removed and cells were washed once in PBS (Gibco, UK). Cells were lysed in 1× NuPAGE lysis buffer (Life Technologies) supplemented with 0.1 M dithiothreitol (DTT) and 250 units/mL benzonase (Sigma). Lysate proteins were separated by SDS-PAGE and transferred to nitrocellulose membranes. Membranes were probed with a primary antibody specific for SNAP-25 (Sigma #S9684) which recognizes uncleaved SNAP—as well as SNAP-25 cleaved by the BoNT/A endopeptidase. The secondary antibody used was an HRP-conjugated anti-rabbit IgG (Sigma #A6154). Bands were detected by enhanced chemiluminescence and imaged using a pXi6 Access (Synoptics, UK). The intensity of bands was determined using GeneTools software (Syngene, Cambridge, UK) and the percentage of SNAP-25 cleaved at each concentration of BoNT calculated. Data were fitted to a 4-parameter logistic equation and pEC₅₀ calculated using GraphPad Prism version 6 (GraphPad).

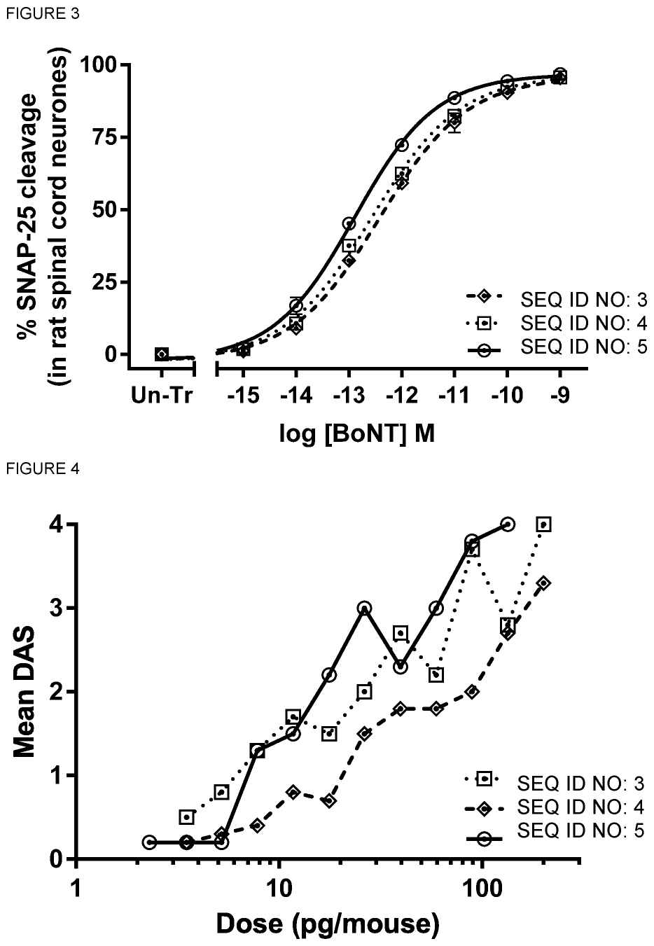

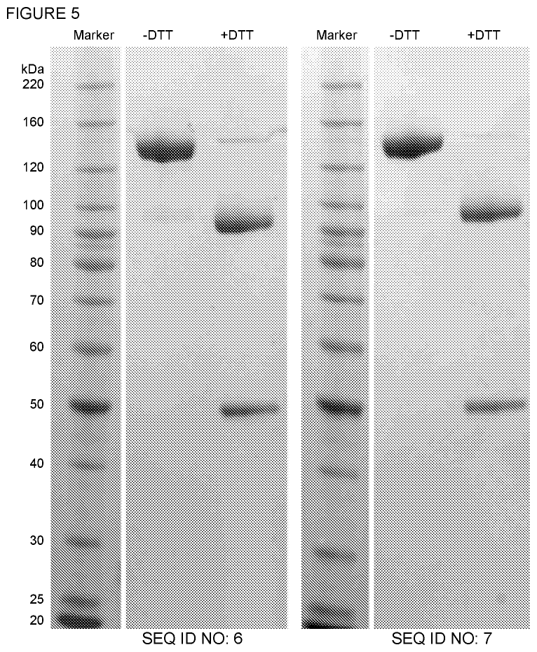

Table 2 below provides the pEC₅₀ values determined for Chimera 1, 2 and 3A in the rat SCN SNAP-25 cleavage assay. These results show that the three BoNT/AB chimeras retained the ability to enter rat spinal cord neurons and cleave their target substrate. However, chimera 3A was more potent than chimera 1 and 2 in this assay (see also FIG. 3).

TABLE 2
pEC₅₀ values.

| | pEC₅₀ ± SEM |
|---|---|
| Chimera 1 | 12.42 ± 0.04 |
| Chimera 2 | 12.57 ± 0.01 |
| Chimera 3A | 12.89 ± 0.04 |

### Digit Abduction Scoring (Das) Assay

The method to measure the activity of BoNT/AB chimera 1, 2 and 3A in the DAS assay is based on the startled response toe spreading reflex of mice, when suspended briefly by the tail. This reflex is scored as Digit Abduction Score (DAS) and is inhibited after administration of BoNT into the gastrocnemius-soleus muscles of the hind paw. Mice are suspended briefly by the tail to elicit a characteristic startled response in which the animal extends its hind limb and abducts its hind digits. (Aoki et al. 1999, Eur. J. Neurol.; 6 (suppl. 4) S3-S10).

On the day of injection, mice were anaesthetized in an induction chamber receiving isoflurane 3% in oxygen. Each mouse received an intramuscular injection of BoNT/AB chimera or vehicle (phosphate buffer containing 0.2% gelatine) in the gastrocnemius-soleus muscles of the right hind paw.

Following neurotoxin injection, the varying degrees of digit abduction were scored on a scale from zero to four, where 0=normal and 4=maximal reduction in digit abduction and leg extension. ED₅₀ was determined by nonlinear adjustment analysis using average of maximal effect at each dose. The mathematical model used was the 4 parameters logistic model.

DAS was performed every 2 hours during the first day after dosing; thereafter it was performed 3 times a day for 4 days.

FIG. 4 shows the fitted curves for chimera 1, 2 and 3A (SEQ ID NO: 3, 4 and 5 converted into a di-chain form, respectively). The chimera 3A curve is shifted to the left, meaning lower doses of chimera 3A achieved a similar DAS response compared to chimera 1 and 2, therefore showing that chimera 3A is more potent than the others in the mouse DAS assay; see also the table below (Table 3) that provides the values for the calculated ED₅₀ and the dose leading to DAS 4 (highest score) for each chimera.

Table 3 below provides the ED₅₀ and DAS 4 doses determined for recombinant BoNT/A1 (rBoNT/A1-SEQ ID NO: 2 converted into a di-chain form) and chimeras 1, 2 and 3A in the mouse DAS assay. These results show that of the three chimeras, chimera 3A has the highest in vivo potency in inducing muscle weakening. Studies shown in FIG. 4 and Table 3 were performed in mice obtained from Charles River laboratories.

TABLE 3
ED₅₀ values.

| | ED₅₀ (pg/mouse) | DAS 4 dose (pg/mouse) |
|---|---|---|
| rBoNT/A1 | 1 | 5 |
| Chimera 1 | 23 | 200 |
| Chimera 2 | 89 | >300 |
| Chimera 3A | 18 | 133 |

### Example 3

### Comparison of BoNT/AB Chimera 3B, 3C and Unmodified BoNT/A1

Untagged BoNT/AB chimera 3B and 3C, respectively with and without the presence of the E1191M/S1199Y double mutation (SEQ ID NO: 6 and 7) were purified as described in Example 1 (FIG. 2), and tested for functional activity using unmodified BoNT/A (SEQ ID NO: 2 converted into a di-chain form) as a reference.

### Human Pluripotent Stem Cells Snap-25 Cleavage Assay

Cryopreserved PERI.4U-cells were purchased from Axiogenesis (Cologne, Germany). Thawing and plating of the cells were performed as recommended by the manufacturer. Briefly, cryovials containing the cells were thawed in a water bath at 37° C. for 2 minutes. After gentle resuspension the cells were transferred to a 50 ml tube. The cryovial was washed with 1 mL of Peri.4UR thawing medium supplied by the manufacturer and the medium was transferred drop-wise to the cell suspension to the 50 ml tube, prior to adding a further 2 mL of Peri.4UR thawing medium drop-wise to the 50 mL tube. Cells were then counted using a hemocytometer. After this, a further 6 mL of Peri.4U® thawing medium was added to the cell suspension. A cell pellet was obtained by centrifugation at 260×g (e.g. 1,100 RPM) for 6 minutes at room temperature. Cells were then resuspended in complete Peri.4UR culture medium supplied by the manufacturer. Cells were plated at a density of 50,000 to 150,000 cells per cm² on cell culture plates coated with poly-L-ornithine and laminin. Cells were cultured at 37° C. in a humidified CO₂ atmosphere, and medium was changed completely every 2-3 days during culture.

For toxin treatment, serial dilutions of BoNTs were prepared in Peri.4UR culture medium. The medium from the wells to be treated was collected and filtered (0.2 μm filter). 125 μL of the filtered medium was added back to each test well. 125 μL of diluted toxin was then added to the plate (triplicate wells). The treated cells were incubated at 37° C., 10% CO₂, for 48+1 h).

### Analysis of BONT Activity Using the SNAP-25 Cleavage Assay

Following treatment, BoNT was removed and cells were washed once in PBS (Gibco, UK). Cells were lysed in 1× NuPAGE lysis buffer (Life Technologies) supplemented with 0.1 M dithiothreitol (DTT) and 250 units/mL benzonase (Sigma). Lysate proteins were separated by SDS-PAGE and transferred to nitrocellulose membranes. Membranes were probed with a primary antibody specific for SNAP-25 (Sigma #S9684) which recognizes uncleaved SNAP—as well as SNAP-25 cleaved by the BoNT/A endopeptidase. The secondary antibody used was an HRP-conjugated anti-rabbit IgG (Sigma #A6154). Bands were detected by enhanced chemiluminescence and imaged using a pXi6 Access (Synoptics, UK). The intensity of bands was determined using GeneTools software (Syngene, Cambridge, UK) and the percentage of SNAP-25 cleaved at each concentration of BoNT calculated. Data were fitted to a 4-parameter logistic equation and pEC₅₀ calculated using GraphPad Prism version 6 (GraphPad).

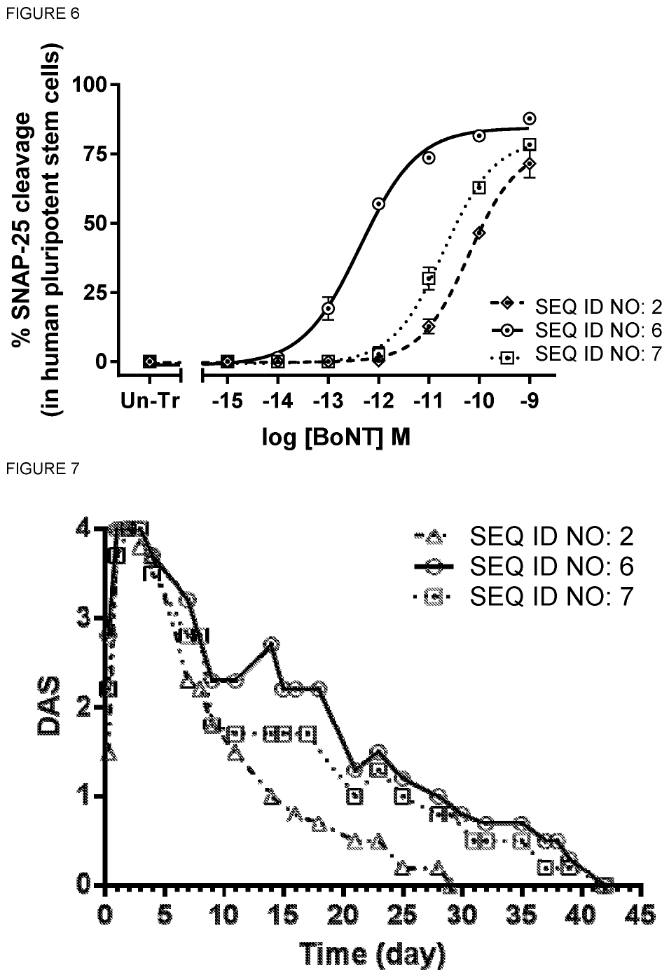

FIG. 6 shows that chimera 3B and 3C displayed greater potency than rBoNT/A1 in cleaving SNAP-25 in induced human pluripotent stem cells but the former significantly more so. This can be explained by the double mutation which increases the affinity of chimera 3B for the human synaptotagmin II protein receptor present in these cells (FIG. 6, Table 4).

TABLE 4
pEC₅₀ values.

| | pEC₅₀ ± SEM |
|---|---|
| rBoNT/A1 | 10.21 ± 0.05 |
| Chimera 3B | 12.38 ± 0.06 |
| Chimera 3C | 10.72 ± 0.08 |

### Digit Abduction Scoring (DAS) Assay—Safety Ratio

The method to measure the activity of BoNTs in the DAS assay is based on the startled response toe spreading reflex of mice, when suspended briefly by the tail. This reflex is scored as Digit Abduction Score (DAS) and is inhibited after administration of BONT into the gastrocnemius-soleus muscles of the hind paw. Mice are suspended briefly by the tail to elicit a characteristic startled response in which the animal extends its hind limb and abducts its hind digits. (Aoki et al. 1999, Eur. J. Neurol.; 6 (suppl. 4) S3-S10).

On the day of injection, mice were anaesthetized in an induction chamber receiving isoflurane 3% in oxygen. Each mouse received an intramuscular injection of BONT or vehicle (phosphate buffer containing 0.2% gelatine) in the gastrocnemius-soleus muscles of the right hind paw.

Following neurotoxin injection, the varying degrees of digit abduction were scored on a scale from zero to four, where 0=normal and 4=maximal reduction in digit abduction and leg extension. ED₅₀ was determined by nonlinear adjustment analysis using average of maximal effect at each dose. The mathematical model used was the 4 parameters logistic model.

DAS was performed every 2 hours during the first day after dosing; thereafter it was performed 3 times a day for 4 days for all doses. Animals of the groups injected with vehicle and the lowest dose that induced during the first four days of injection a DAS of 4 were thereafter monitored until complete recovery of the muscle weakness to a DAS of 0 (no observed muscle weakness).

For calculation of the safety ratio all animals were weighed the day before toxin injection (DO) and thereafter once daily throughout the duration of the study. The average body weight, its standard deviation, and the standard error mean were calculated daily for each dose-group. To obtain the safety ratio for a BoNT (—10% ΔBW/ED₅₀), the dose at which at any time during the study the average weight of a dose-group was lower than 10% of the average weight at DO of that same dose-group was divided by the ED₅₀ for the BoNT studied. The lethal dose was defined as the dose at which one or more of the animals within that dose-group died.

FIG. 7 shows the duration of muscle weakening over time in the mouse digit abduction scoring assay for unmodified BoNT/A, chimera 3B and chimera 3C (SEQ ID NO: 2, 6 and 7 converted into a di-chain form), showing that the chimera has longer duration of action.

Table 5 below provides the ED₅₀ and DAS 4 doses determined for rBoNT/A1 and chimeras 3B and 3C in the mouse DAS assay. The table also provide the total duration of action for the DAS 4 dose until complete recovery of the muscle weakness to a DAS of 0 (no observed muscle weakness). In addition, the table shows the mouse lethal dose and the safety ratio (−10% ΔBW/ED₅₀), as defined in the text above. In comparison to rBoNT/A1, chimeras 3B and 3C have longer duration of action, a better safety ratio, and a higher lethal dose. Studies shown in FIG. 7 and Table 5 were performed in mice obtained from Janvier laboratories.

TABLE 5
DAS and Safety Ratios of the BoNT/AB chimeras.

| | | | | | Safety |
|---|---|---|---|---|---|
| | ED₅₀ | DAS 4 | Total duration | Mouse | ratio |
| | (DAS 2) | dose | of action (day) | lethal | (−10% |
| | Dose (pg/ | (pg/ | with lowest | dose | ΔBW/ |
| | mouse) | mouse) | DAS 4 dose | (pg) | ED₅₀) |
| rBoNT/A1 | 0.9 | 2.3 | 29 | 18 | 4.5 |
| Chimera 3B | 8.0 | 89 | 42 | 200 | 14.1 |
| Chimera 3C | 5.0 | 26 | 42 | 8.9 | 7.4 |

### Example 4

Pre-Clinical Testing of Modified BoNT/a (BoNT/AB Chimera [SEQ ID NO: 6 Converted into a Di-Chain Form])

BONT/AB chimera SEQ ID NO: 6 converted into a di-chain form was tested in a mouse LD₅₀ assay yielding a result of 1.202 ng/kg. 1 Unit of SEQ ID NO: 6 therefore corresponds to 24.04 pg in this assay.

Additionally, said BoNT/AB chimera was tested in a rat DAS assay to determine the duration of action when compared to Dysport®. Results are presented in Table 6 below:

TABLE 6
Duration of action.

| | Dysport ® | BoNT/AB |
|---|---|---|
| | 3 U/rat | 300 pg/rat |
| | 15 U/kg | 1.5 ng/kg |
| Duration of Action (median days) | 21.9 | 47.7 |

In conclusion, the duration of action of BoNT/AB was much higher than Dysport®.

### Example 5

Determination of a Unit Dose of Modified BoNT/a (SEQ ID NO: 6 Converted into a Di-Chain Form) for Treating Cervical Dystonia

In view of pre-clinical pharmacology data, a suitable unit dose range (UD) for administration of modified BoNT/A in humans has been determined.

A DAS ED₅₀ of 13 pg/kg was calculated for SEQ ID NO: 6. ED₅₀ is considered as a minimal pharmacologically active dose, which is approximately 300-fold lower than the no observed adverse effect level (NOAEL) of 4 ng/kg in the same animal species. An ED₅₀ of 13 pg/kg of SEQ ID NO: 6 in rats corresponds to a 0.8 ng dose for a human of 60 kg body weight.

Thus, the lower limit of a unit dose of 1,000 pg was selected. An upper limit of the unit dose of 16,000 pg was selected, which is lower than the NOAEL of 4 ng/kg from both nonclinical safety species (rat and monkey) converted into human dose for 60 kg body weight. Thus, a unit dose was determined to be 1,000 pg to 16,000 pg (˜42 Units to ˜666 Units).

In view of the improved safety profile the maximum total dose for the treatment of cervical dystonia was set at 160,000 pg (˜7,070 Units), which is derived from the NOAEL of 4 ng/kg from both nonclinical safety species (rat and monkey) converted into human dose for 60 kg body weight.

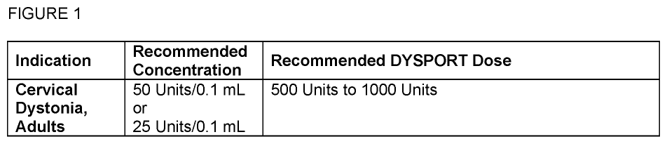

In view of the improved safety profile when compared to Dysport® as determined by the pre-clinical data of Example 4, total dosages (in units) administered in cervical dystonia are expected to be almost 7x greater than that for Dysport®. The maximum total dose of Dysport® for treatment of cervical dystonia is 1,000 Units (see FIG. 1).

Advantageously, more modified BoNT/A (SEQ ID NO: 6) can be injected and/or can be injected at a greater number of neck muscles/sites in the treatment of cervical dystonia before reaching the maximum dose. This is a significant and advantageous finding leading to improved treatment of cervical dystonia while providing clinicians with a greater range of treatment options.

### Example 6

Dosage Regimen for Treating Cervical Dystonia Using a Modified BoNT/a (SEQ ID NO: 6 Converted into a Di-Chain Form)

Modified BoNT/A (e.g. SEQ ID NO: 6 converted into a di-chain form) is provided as a lyophilised powder in a vial containing 36 ng of modified BoNT/A per vial. The lyophilised powder is reconstituted.

The unit dose (UD) is 1,000-16,000 pg (˜42-666 Units [measured by mouse LD₅₀]).

Cervical dystonia is treated by intramuscular injection according to the following dosage regimen (Table 7):

TABLE 7
Dosage regimen.

| Neck Muscle | Dosage (Unit Dose) |
|---|---|
| Sternocleidomastoid | 1 × UD |
| Splenius capitis | 1 × UD |
| Splenius cervicis | 1 × UD |
| Trapezius | 1 × UD |
| Levator scapulae | 1 × UD |
| Scalenus medius | 1 × UD |
| Scalenus anterior | 1 × UD |
| Semispinalis capitis | 1 × UD |
| Longissimus | 1 × UD |
| Posterior paravertebrals | 1 × UD |
| Submental complex | 1 × UD |

The administration may be unilateral or bilateral as required based on the specific presentation.

A maximum total dosage administered is 10× UD (e.g. in some cases 2× UD are administered to one or more of the neck muscles indicated). This corresponds to 160,000 pg/˜6,660 Units. This is almost 7× greater than the maximum total dosage of Dysport® that can be administered during treatment of cervical dystonia without approaching toxic limits (a concern with conventional treatment regimens). Thus, the clinician is able to tailor treatment to the patient with the knowledge that 10× UD can be administered without any concern of toxicity, thereby allowing the treatment of additional neck muscles of the subject and/or ensuring each neck muscle receives a pharmaceutically effective dose.

### Example 7

Treatment of a Patient with Cervical Dystonia (Laterocollis)

Jane, aged 65, is diagnosed by her GP with cervical dystonia. The specific presentation is as laterocollis. A single unit dose (3,000 pg) of modified BoNT/A (SEQ ID NO: 6 converted into a di-chain form) is ipsilaterally administered to Jane's levator scapulae muscle and a single unit dose is also ipsilaterally administered to Jane's sternocleidomastoideole (resulting in a total dose at the treatment session of 6,000 pg). The laterocollis is alleviated and, owing to the long duration of the modified BoNT/A, Jane does not require further treatment for greater than 9 months. Thus, Jane receives less frequent injections (e.g. per year) when compared to an equivalent subject administered an unmodified BoNT/A. Additionally, Jane does not exhibit any side-effects owing to the improved safety profile of the modified BoNT/A.

### Example 8

Treatment of a Patient with Cervical Dystonia (Retrocollis)

Brian, aged 48, is diagnosed by his GP with cervical dystonia. The specific presentation is as retrocollis. Modified BoNT/A (SEQ ID NO: 6 converted into a di-chain form) is administered to each of the following of Brian's muscles:
1× unit dose (UD) of 10,000 pg to each levator scapulae;
1× UD of 10,000 pg to each trapezius;
1× UD of 10,000 pg to each longissimus;
1× UD of 10,000 pg to each splenius capitis; and
1× UD of 10,000 pg to each splenius cervicis.

The total dose administered is 10× UDs (100,000 pg), which is well-within the upper limit of 160,000 pg and is possible given the greater safety profile of the modified BoNT/A when compared to an unmodified BoNT/A. The retrocollis is alleviated and, owing to the long duration of the modified BoNT/A, Brian does not require further treatment for 12 months. Thus, Brian receives less frequent injections when compared to an equivalent subject administered an unmodified BoNT/A.

### Example 9

Safety & Efficacy of Modified BoNT/a (SEQ ID NO: 6 Converted into a Di-Chain Form) in Humans

SEQ ID NO: 6 (converted into a di-chain form) was administered to human subjects by way of a single unit dose of modified BoNT/A. 5 cohorts were administered different (increasing) amounts of modified BoNT/A (SEQ ID NO: 6 converted into a di-chain form). Cohort 1 was administered 2×1,000 pg unit doses of modified BoNT/A (i.e. 2,000 pg maximum), while cohort was administered 2×16,000 pg unit doses of modified BoNT/A (i.e. 32,000 pg maximum).

Results showed that all unit doses of modified BoNT/A tested, (i.e. up to 16,000 pg unit doses), were effective at muscle paralysis, safely tolerated, and no adverse effects were observed, despite the exceptionally high dosage per muscle. This shows that the modified BoNT/A does not diffuse away from the injection site and highlights the exceptional safety profile of modified BONT/A (SEQ ID NO: 6 converted into a di-chain form).

### Example 10

Dosage Regimen for Treating Cervical Dystonia Using a Modified BoNT/a (SEQ ID NO: 6 Converted into a Di-Chain Form)

Modified BoNT/A (e.g. SEQ ID NO: 6 converted into a di-chain form) is provided as a lyophilised powder in a vial containing 36 ng of modified BoNT/A per vial. The lyophilised powder is reconstituted.

The unit dose (UD) is 17,000-36,000 pg (˜707-1,498 Units [measured by mouse LD₅₀]).

Cervical dystonia is treated by intramuscular injection according to the following dosage regimen (Table 8):

TABLE 8
Dosage regimen.

| Neck Muscle | Dosage (Unit Dose) |
|---|---|
| Sternocleidomastoid | 1 × UD |
| Splenius capitis | 1 × UD |
| Splenius cervicis | 1 × UD |
| Trapezius | 1 × UD |
| Levator scapulae | 1 × UD |
| Scalenus medius | 1 × UD |
| Scalenus anterior | 1 × UD |
| Semispinalis capitis | 1 × UD |
| Longissimus | 1 × UD |
| Posterior paravertebrals | 1 × UD |
| Submental complex | 1 × UD |

The administration may be unilateral or bilateral as required based on the specific presentation.

A maximum total dosage administered is 10x UD (e.g. in some cases 2× UD are administered to one or more of the neck muscles indicated). This corresponds to 360,000 pg/˜14,975 Units. This is over 20× greater than the maximum total dosage of Dysport® that can be administered during treatment of cervical dystonia without approaching toxic limits (a concern with conventional treatment regimens). Thus, the clinician is able to tailor treatment to the patient with the knowledge that 10× UD can be administered without any concern of toxicity, thereby allowing the treatment of additional neck muscles of the subject and/or ensuring each neck muscle receives a pharmaceutically effective dose.

### Example 11

Treatment of a Patient with Cervical Dystonia (Laterocollis)

Elizabeth, aged 62, is diagnosed by her GP with cervical dystonia. The specific presentation is as laterocollis. A single unit dose (36,000 pg) of modified BoNT/A (SEQ ID NO: 6 converted into a di-chain form) is ipsilaterally administered to Elizabeth's levator scapulae muscle and a single unit dose is also ipsilaterally administered to Elizabeth's sternocleidomastoideole (resulting in a total dose at the treatment session of 72,000 pg). The laterocollis is alleviated and, owing to the long duration of the modified BoNT/A, Elizabeth does not require further treatment for greater than 9 months. Thus, Elizabeth receives less frequent injections (e.g. per year) when compared to an equivalent subject administered an unmodified BoNT/A. Additionally, Elizabeth does not exhibit any side-effects owing to the improved safety profile of the modified BoNT/A.

### Example 12

Treatment of a Patient with Cervical Dystonia (Retrocollis)

Donald, aged 54, is diagnosed by his GP with cervical dystonia. The specific presentation is as retrocollis. Modified BoNT/A (SEQ ID NO: 6 converted into a di-chain form) is administered bilaterally to each of the following of Donald's muscles:
1× unit dose (UD) of 36,000 pg to each levator scapulae;
1× UD of 36,000 pg to each trapezius;
1× UD of 36,000 pg to each longissimus;
1× UD of 36,000 pg to each splenius capitis; and
1× UD of 36,000 pg to each splenius cervicis.

The total dose administered is 10x UDs (360,000 pg), which is possible given the greater safety profile of the modified BoNT/A when compared to an unmodified BoNT/A. The retrocollis is alleviated and, owing to the long duration of the modified BoNT/A, Donald does not require further treatment for 12 months. Thus, Donald receives less frequent injections when compared to an equivalent subject administered an unmodified BoNT/A. Additionally, Donald does not exhibit any side effects owing to the improved safety profile of the modified BoNT/A.

### Example 13

Safety & Efficacy of Modified BoNT/a (SEQ ID NO: 6 Converted into a Di-Chain Form) in Humans

SEQ ID NO: 6 (converted into a di-chain form) was administered to human subjects by way of intramuscular injection. Subjects were administered 2× of a 15,000 pg unit dose (i.e. 30,000 pg total), 2× of a 25,000 pg unit dose (i.e. 50,000 pg total), or 2× of a 36,000 pg unit dose (i.e. 72,000 pg total) of modified BoNT/A (SEQ ID NO: 6 converted into a di-chain form).

Results showed that all unit doses of modified BoNT/A tested were effective at muscle paralysis, safely tolerated, and no adverse effects were observed, despite the exceptionally high dosage per muscle (e.g. for the 25,000 pg and 36,000 pg unit dose). This shows that the modified BoNT/A does not diffuse away from the injection site and highlights the exceptional safety profile of modified BoNT/A (SEQ ID NO: 6 converted into a di-chain form).

All publications mentioned in the above specification are herein incorporated by reference. Various modifications and variations of the described methods and system of the present invention will be apparent to those skilled in the art without departing from the scope and spirit of the present invention. Although the present invention has been described in connection with specific preferred embodiments, it should be understood that the invention as claimed should not be unduly limited to such specific embodiments. Indeed, various modifications of the described modes for carrying out the invention which are obvious to those skilled in biochemistry and biotechnology or related fields are intended to be within the scope of the following claims.

1. A modified botulinum neurotoxin A (BoNT/A) for use in treating cervical dystonia, wherein the modified BoNT/A is administered by intramuscular injection to an affected neck muscle of a subject,
wherein the modified BoNT/A is administered by way of a unit dose of greater than 17,000 pg of modified BoNT/A,
wherein at least a single unit dose is administered to the affected neck muscle,
wherein the total dose administered during the treatment is up to 400,000 pg of modified BoNT/A, and
wherein the modified BoNT/A comprises a BoNT/A light-chain and translocation domain, and a BoNT/B receptor binding domain (H_{C} domain).

2. A method for treating cervical dystonia, the method comprising administering a modified BoNT/A by intramuscular injection to an affected neck muscle of a subject,
wherein the modified BoNT/A is administered by way of a unit dose of greater than 17,000 pg of modified BoNT/A,
wherein at least a single unit dose is administered to the affected neck muscle,
wherein the total dose administered during the treatment is up to 400,000 pg of modified BoNT/A, and
wherein the modified BoNT/A comprises a BoNT/A light-chain and translocation domain, and a BoNT/B receptor binding domain (H_{C} domain).

3. Use of a modified botulinum neurotoxin A (BoNT/A) in the manufacture of a medicament for treating cervical dystonia, wherein the modified BoNT/A is administered by intramuscular injection to an affected neck muscle of a subject,
wherein the modified BoNT/A is administered by way of a unit dose of greater than 17,000 pg of modified BoNT/A,
wherein at least a single unit dose is administered to the affected neck muscle,
wherein the total dose administered during the treatment is up to 400,000 pg of modified BoNT/A, and
wherein the modified BoNT/A comprises a BoNT/A light-chain and translocation domain, and a BoNT/B receptor binding domain (H_{C} domain).

4. The modified BoNT/A for use, method, or the use according to any one of the preceding claims, wherein the total dose administered is 170,000 pg to 400,000 pg (e.g. greater than 170,000 pg up to 400,000 pg) of modified BoNT/A.

5. The modified BoNT/A for use, method, or the use according to any one of claims 1-4, wherein the total dose administered is 170,000 pg up to 360,000 pg (e.g. greater than 170,000 pg up to 360,000 pg) of modified BoNT/A

6. The modified BoNT/A for use, method, or the use according to any one of claims 1-4, wherein the total dose administered is 250,000 pg to 400,000 pg (e.g. 250,000 pg up to 360,000 pg) of modified BoNT/A.

7. The modified BoNT/A for use, method, or the use according to any one of claims 1-5, wherein the total dose administered is 200,000 pg to 300,000 pg, such as 240,000 to 260,000 pg (e.g. 250,000 pg) of modified BoNT/A.

8. The modified BoNT/A for use, method, or the use according to any one of claim 1-4 or 6, wherein the total dose administered is 300,000 pg to up to 400,000 pg, such as 350,000 pg to 370,000 pg, (e.g. 360,000 pg) of modified BoNT/A.

9. The modified BoNT/A for use, method, or use according to any one of the preceding claims, wherein the total dose administered during the treatment is up to 250,000 pg of modified BoNT/A.

10. The modified BoNT/A for use, method, or use according to any one of the preceding claims, wherein the total dose administered during the treatment is up to 360,000 pg of modified BoNT/A.

11. The modified BoNT/A for use, method, or use according to any one of the preceding claims, wherein the total dose administered during the treatment is at least 250,000 pg of modified BoNT/A.

12. The modified BoNT/A for use, method, or use according to any one of the preceding claims, wherein the unit dose is greater than 17,000 pg up to 40,000 pg (e.g. 17,001 pg up to 40,000 pg) of modified BoNT/A.

13. The modified BoNT/A for use, method, or use according to any one of the preceding claims, wherein the unit dose is greater than 17,000 pg up to 36,000 pg (e.g. 17,001 pg up to 36,000 pg) of modified BoNT/A.

14. The modified BoNT/A for use, method, or use according to any one of claims 1-2, wherein the unit dose is 25,000 pg up to 40,000 pg (e.g. 25,000 pg to 36,000 pg) of modified BONT/A.

15. The modified BoNT/A for use, method, or use according to any one of claim 1-12 or 14, wherein the total dose administered is 30,000 pg to up to 40,000 pg, such as 35,000 pg to 37,000 pg, (preferably 36,000 pg) of modified BoNT/A.

16. The modified BoNT/A for use, method, or use according to any one of claims 1-13, wherein the unit dose is 20,000 pg to 30,000 pg, such as 24,000 to 26,000 pg (preferably 25,000 pg) of modified BoNT/A.

17. A modified botulinum neurotoxin A (BoNT/A) for use in treating cervical dystonia, wherein the modified BoNT/A is administered by intramuscular injection to an affected neck muscle of a subject,
wherein the modified BoNT/A is administered by way of a unit dose of greater than 707 U of modified BoNT/A, wherein 1 Unit is an amount of the modified BoNT/A that corresponds to the calculated median lethal dose (LD₅₀) in mice,
wherein at least a single unit dose is administered to the affected neck muscle,
wherein the total dose administered during the treatment is up to 16,639 U of modified BONT/A, and
wherein the modified BoNT/A comprises a BoNT/A light-chain and translocation domain, and a BoNT/B receptor binding domain (H_{C} domain).

18. A method for treating cervical dystonia, the method comprising administering a modified BoNT/A by intramuscular injection to an affected neck muscle of a subject, wherein the modified BoNT/A is administered by way of a unit dose of greater than 707 U of modified BoNT/A, wherein 1 Unit is an amount of the modified BoNT/A that corresponds to the calculated median lethal dose (LD₅₀) in mice,
wherein at least a single unit dose is administered to the affected neck muscle,
wherein the total dose administered during the treatment is up to 16,639 U of modified BONT/A, and
wherein the modified BoNT/A comprises a BoNT/A light-chain and translocation domain, and a BoNT/B receptor binding domain (H_{C} domain).

19. Use of a modified botulinum neurotoxin A (BoNT/A) in the manufacture of a medicament for treating cervical dystonia, wherein the modified BoNT/A is administered by intramuscular injection to an affected neck muscle of a subject,
wherein the modified BoNT/A is administered by way of a unit dose of greater than 707 Units of modified BoNT/A, wherein 1 Unit is an amount of the modified BoNT/A that corresponds to the calculated median lethal dose (LD₅₀) in mice,
wherein at least a single unit dose is administered to the affected neck muscle,
wherein the total dose administered during the treatment is up to 16,639 U of modified BONT/A, and
wherein the modified BoNT/A comprises a BoNT/A light-chain and translocation domain, and a BoNT/B receptor binding domain (H_{C} domain).

20. The modified BoNT/A for use, method, or use according to any one of claims 17-19, wherein the total dose administered is 7,072 Units up to 16,639 Units (e.g. greater than 7,072 Units up to 16,639 Units) of modified BoNT/A.

21. The modified BoNT/A for use, method, or use according to any one of claims 17-20, wherein the total dose administered is 10,399 Units to 16,639 Units (e.g. 10,399 Units up to 14,975 Units) of modified BoNT/A.

22. The modified BoNT/A for use, method, or use according to any one of claims 17-20, wherein the total dose administered is 8,319 Units to 12,479 Units, such as 9,983 Units to 10,815 Units (e.g. 10,399 Units) of modified BoNT/A.

23. The modified BoNT/A for use, method, or use according to any one of claims 17-21, wherein the total dose administered is 12,479 Units to 16,639 Units, such as 14,559 Units to 15,391 Units (e.g. 14,975 Units) of modified BoNT/A.

24. The modified BoNT/A for use, method, or use according to any one of claims 17-23, wherein the total dose administered during the treatment is up to 14,975 U of modified BONT/A.

25. The modified BoNT/A for use, method, or use according to any one of claims 17-22, wherein the total dose administered during the treatment is up to 10,399 Units.

26. The modified BoNT/A for use, method, or use according to any one of claims 17-25, wherein the unit dose is greater than 707 Units up to 1,664 Units (e.g. 707.2 or 708 Units up to 1,664 Units) of modified BoNT/A.

27. The modified BoNT/A for use, method, or use according to any one of claims 17-26, wherein the unit dose is greater than 707 Units up to 1,498 Units (e.g. 707.2 or 708 Units up to 1,498 Units) of modified BoNT/A.

28. The modified BoNT/A for use, method, or use according to any one of claims 17-26, wherein the unit dose is 1,040 Units up to 1,664 Units (e.g. 1,040 Units to 1,498 Units) of modified BoNT/A.

29. The modified BoNT/A for use, method, or use according to any one of claims 17-27, wherein the unit dose is 832 Units to 1,248 Units, such as 998 Units to 1,082 Units (preferably 1,040 Units) of modified BoNT/A.

30. The modified BoNT/A for use, method, or use according to any one of claim 17-26 or 28, wherein the unit dose is 1,248 Units to 1,664 Units, such as 1,456 Units to 1,539 Units, (preferably 1,498 Units) of modified BoNT/A.

31. The modified BoNT/A for use, method or use according to any one of the preceding claims, wherein the modified BoNT/A comprises a polypeptide sequence having at least 70% sequence identity to SEQ ID NO: 6, or comprises the polypeptide sequence of SEQ ID NO: 6, or consists of the polypeptide sequence of SEQ ID NO: 6.

32. The modified BoNT/A for use, method, or use according to any one of the preceding claims, wherein the modified BoNT/A is a di-chain modified BoNT/A in which the L-chain is linked to the H-chain via a di-sulphide bond obtainable by a method comprising contacting a single-chain modified BoNT/A consisting of SEQ ID NO: 6 with a protease that hydrolyses a peptide bond in the activation loop thereof, thereby converting the single-chain modified BoNT/A into the corresponding di-chain modified BoNT/A.

33. The modified BoNT/A for use, method, or use according to any one of the preceding claims, wherein an initial methionine amino acid residue of a polypeptide sequence of the modified BoNT/A is optional.

34. The modified BoNT/A for use, method, or use according to any one of the preceding claims, wherein an initial methionine amino acid residue of a polypeptide sequence of the modified BoNT/A is absent.

35. The modified BoNT/A for use, method, or use according to any one of the preceding claims, wherein the modified BoNT/A is a di-chain modified BoNT/A comprising (or consisting of) a light-chain comprising SEQ ID NO: 11 or 12 (preferably SEQ ID NO: 11) and a heavy-chain comprising SEQ ID NO: 13, wherein the light-chain and heavy-chain are joined together by a di-sulphide bond.

36. The modified BoNT/A for use, method, or use according to any one of the preceding claims, wherein the modified BoNT/A has a Safety Ratio of greater than 7, wherein the Safety Ratio is calculated as: dose of toxin required for-10% bodyweight change measured as pg/mouse divided by DAS ED₅₀ measured as pg/mouse, wherein ED₅₀=dose required to produce a DAS score of 2.

37. The modified BoNT/A for use, method, or use according to any one of the preceding claims, wherein the affected neck muscle is selected from: the sternocleidomastoide sternocleidomastoideus, the splenius capitis, the splenius cervicis, the scalene complex (e.g. the scalenus anterior and/or the scalenus medius), the trapezius (e.g. the upper trapezius), the levator scapulae, the semispinalis capitis, the longissimus (e.g. longissimus capitis and/or longissimus cervicis), the posterior paravertebrals and the submental complex (e.g. the digastric muscle, the geniohyoid muscle, the mylohyoid muscle, the mylohyoid boutonniere and/or the stylohyoid muscle), the trapezius pars descendens, the linea nuchalis superior—Clavicula (lateral part), the processus spinosus C3-Th3—processus mastoideus, the processus spinosus Th3-Th5—processus transversus C1-C2, the processus transversus C3-Th6, the processus spinosus C3-Th1—linea nuchalis superior, the processus transversus Th1-Th6—processus spinosus C2-C7, the processus transversus C3-Th3—processus mastoideus, the processus transversus Th1-Th6—processus transversus C2-C6, the obliquus capitis inferior, the processus spinosus C2—processus transversus C1, the suprasternal notch and clavicula (medial part)—processus mastoideus and linea nuchalis superior, the processus transversus C1-C4—scapula (angulus superior), the processus transversus C2-C7—first rib, the processus transversus C3-C6—first rib, the longus capitis, the processus transversus C3-C6—occipital bone (basilar part), the longus colli, and the processus transversus C2-C5—atlas (anterior tubercle).

38. The modified BoNT/A for use, method, or use according to any one of the preceding claims, wherein the affected neck muscle is selected from: the sternocleidomastoideus (e.g. the left or right sternocleidomastoideolenius capitis (e.g. left or right splenius capitis), the scalenus anterior, the scalenus medius, the trapezius, (e.g. the left or right trapezius such as the left or right upper trapezius), the levator scapulae (e.g. left or right levator scapulae), the semispinalis capitis (e.g. the left or right semispinalis capitis), the semispinalis capitis pars med, the longissimus (e.g. the left or right longissimus capitis and/or longissimus cervicis), the splenius cervicis, the scalene complex (e.g. the scalenus anterior and/or the scalenus medius), the posterior paravertebrals (e.g. the scalenus posterior, scalenus medius and/or scalenus anterior, preferably the scalenus posterior), the submental complex (e.g. the digastric muscle, the geniohyoid muscle, the mylohyoid muscle, the mylohyoid boutonniere and/or the stylohyoid muscle), the trapezius pars descendens, the linea nuchalis superior—Clavicula (lateral part), the processus spinosus C3-Th3—processus mastoideus, the processus spinosus Th3-Th5—processus transversus C1-C2, the processus transversus C3-Th6, the processus spinosus C3-Th1—linea nuchalis superior, the processus transversus Th1-Th6—processus spinosus C2-C7, the processus transversus C3-Th3—processus mastoideus, the processus transversus Th1-Th6—processus transversus C2-C6, the obliquus capitis inferior, the obliquus capitis superior, the processus spinosus C2—processus transversus C1, the suprasternal notch and clavicula (medial part)—processus mastoideus and linea nuchalis superior, the processus transversus C1-C4—scapula (angulus superior), the processus transversus C2-C7—first rib, the processus transversus C3-C6—first rib, the longus capitis, the processus transversus C3-C6—occipital bone (basilar part), the longus colli, the semispinalis cervicis, the spinalis capitis, the rectus capitis posterior major, the rectus capitis posterior minor, the rectus capitis anterior, the multifudus, and the processus transversus C2-C5—atlas (anterior tubercle).

39. The modified BoNT/A for use, method, or use according to any one of the preceding claims, wherein the affected neck muscle(s) comprise(s) the sternocleidomastoideus, (e.g. the left or right sternocleidomastoid), the splenius capitis (e.g. left or right splenius capitis), the scalenus anterior, the scalenus medius, the trapezius, (e.g. the left or right trapezius such as the left or right upper trapezius), the levator scapulae (e.g. left or right levator scapulae), the semispinalis capitis (e.g. the left or right semispinalis capitis), the semispinalis capitis pars med, the longissimus (e.g. the left or right longissimus capitis and/or longissimus cervicis), the splenius cervicis, the scalene complex (e.g. the scalenus anterior and/or the scalenus medius), the posterior paravertebrals (e.g. the scalenus posterior, scalenus medius and/or scalenus anterior, preferably the scalenus posterior), the submental complex (e.g. the digastric muscle, the geniohyoid muscle, the mylohyoid muscle, the mylohyoid boutonniere and/or the stylohyoid muscle), the trapezius pars descendens, the linea nuchalis superior—Clavicula (lateral part), the processus spinosus C3-Th3—processus mastoideus, the processus spinosus Th3-Th5—processus transversus C1-C2, the processus transversus C3-Th6, the processus spinosus C3-Th1-linea nuchalis superior, the processus transversus Th1-Th6—processus spinosus C2-C7, the processus transversus C3-Th3—processus mastoideus, the processus transversus Th1-Th6—processus transversus C2-C6, the obliquus capitis inferior, the obliquus capitis superior, the processus spinosus C2—processus transversus C1, the suprasternal notch and clavicula (medial part)—processus mastoideus and linea nuchalis superior, the processus transversus C1-C4—scapula (angulus superior), the processus transversus C2-C7—first rib, the processus transversus C3-C6—first rib, the longus capitis, the processus transversus C3-C6—occipital bone (basilar part), the longus colli, the semispinalis cervicis, the spinalis capitis, the rectus capitis posterior major, the rectus capitis posterior minor, the rectus capitis anterior, the multifudus, or the processus transversus C2-C5—atlas (anterior tubercle).

40. The modified BoNT/A for use, method, or use according to any one of the preceding claims, wherein the modified BoNT/A is administered to one or more affected neck muscle(s) selected from or comprising: M. semispinalis cervicis, M. levator scapulae, M. splenius cervicis, M. longissimus cervicis, M. trapezius (e.g. M. trapezius pars descendens), M. sternocleidomastoideus, M. semispinalis capitis, M. obliquus capitis inferior, M. longissimus capitis, M. splenius capitis, an M. scalenus (e.g. M. scalenus anterior, medius, and/or posterior), M. longus colli, and/or M. longus capitis.

41. The modified BoNT/A for use, method, or use according to any one of the preceding claims, wherein the modified BoNT/A is administered by intramuscular injection to a plurality of affected neck muscles of the subject, and wherein at least a single unit dose is administered to each affected neck muscle,
preferably wherein the plurality of affected neck muscles are selected from: the sternocleidomastoideornocleidomastoideus, the splenius capitis, the splenius cervicis, the scalene complex (e.g. the scalenus anterior and/or the scalenus medius), the trapezius (e.g. the upper trapezius), the levator scapulae, the semispinalis capitis, the longissimus (e.g. longissimus capitis and/or longissimus cervicis), the posterior paravertebrals and the submental complex (e.g. the digastric muscle, the geniohyoid muscle, the mylohyoid muscle, the mylohyoid boutonniere and/or the stylohyoid muscle), the trapezius pars descendens, the linea nuchalis superior—Clavicula (lateral part), the processus spinosus C3-Th3—processus mastoideus, the processus spinosus Th3-Th5—processus transversus C1-C2, the processus transversus C3-Th6, the processus spinosus C3-Th1—linea nuchalis superior, the processus transversus Th1-Th6—processus spinosus C2-C7, the processus transversus C3-Th3—processus mastoideus, the processus transversus Th1-Th6—processus transversus C2-C6, the obliquus capitis inferior, the processus spinosus C2—processus transversus C1, the suprasternal notch and clavicula (medial part)—processus mastoideus and linea nuchalis superior, the processus transversus C1-C4—scapula (angulus superior), the processus transversus C2-C7—first rib, the processus transversus C3-C6—first rib, the longus capitis, the processus transversus C3-C6—occipital bone (basilar part), the longus colli, and the processus transversus C2-C5—atlas (anterior tubercle).

42. The modified BoNT/A for use, method, or use according to any one of the preceding claims, wherein the modified BoNT/A is administered by intramuscular injection to a plurality of affected neck muscles of the subject, and wherein at least a single unit dose is administered to each affected neck muscle, wherein the plurality of affected neck muscles are selected from: the sternocleidomastoideus (e.g. the left or right sternocleidomastoid the splenius capitis (e.g. left or right splenius capitis), the scalenus anterior, the scalenus medius, the trapezius, (e.g. the left or right trapezius such as the left or right upper trapezius), the levator scapulae (e.g. left or right levator scapulae), the semispinalis capitis (e.g. the left or right semispinalis capitis), the semispinalis capitis pars med, the longissimus (e.g. the left or right longissimus capitis and/or longissimus cervicis), the splenius cervicis, the scalene complex (e.g. the scalenus anterior and/or the scalenus medius), the posterior paravertebrals (e.g. the scalenus posterior, scalenus medius and/or scalenus anterior, preferably the scalenus posterior), the submental complex (e.g. the digastric muscle, the geniohyoid muscle, the mylohyoid muscle, the mylohyoid boutonniere and/or the stylohyoid muscle), the trapezius pars descendens, the linea nuchalis superior-Clavicula (lateral part), the processus spinosus C3-Th3—processus mastoideus, the processus spinosus Th3-Th5—processus transversus C1-C2, the processus transversus C3-Th6, the processus spinosus C3-Th1—linea nuchalis superior, the processus transversus Th1-Th6—processus spinosus C2-C7, the processus transversus C3-Th3-processus mastoideus, the processus transversus Th1-Th6—processus transversus C2-C6, the obliquus capitis inferior, the obliquus capitis superior, the processus spinosus C2-processus transversus C1, the suprasternal notch and clavicula (medial part)—processus mastoideus and linea nuchalis superior, the processus transversus C1-C4—scapula (angulus superior), the processus transversus C2-C7—first rib, the processus transversus C3-C6—first rib, the longus capitis, the processus transversus C3-C6—occipital bone (basilar part), the longus colli, the semispinalis cervicis, the spinalis capitis, the rectus capitis posterior major, the rectus capitis posterior minor, the rectus capitis anterior, the multifudus, and the processus transversus C2-C5—atlas (anterior tubercle).

43. The modified BoNT/A for use, method, or use according to any one of the preceding claims, wherein the modified BoNT/A is administered by intramuscular injection to a plurality of affected neck muscles of the subject, and wherein at least a single unit dose is administered to each affected neck muscle, wherein the plurality of affected neck muscles comprises: the sternocleidomastoideus (e.g. the left or right sternocleidomastoide splenius capitis (e.g. left or right splenius capitis), the scalenus anterior, the scalenus medius, the trapezius, (e.g. the left or right trapezius such as the left or right upper trapezius), the levator scapulae (e.g. left or right levator scapulae), the semispinalis capitis (e.g. the left or right semispinalis capitis), the semispinalis capitis pars med, the longissimus (e.g. the left or right longissimus capitis and/or longissimus cervicis), the splenius cervicis, the scalene complex (e.g. the scalenus anterior and/or the scalenus medius), the posterior paravertebrals (e.g. the scalenus posterior, scalenus medius and/or scalenus anterior, preferably the scalenus posterior), the submental complex (e.g. the digastric muscle, the geniohyoid muscle, the mylohyoid muscle, the mylohyoid boutonniere and/or the stylohyoid muscle), the trapezius pars descendens, the linea nuchalis superior-Clavicula (lateral part), the processus spinosus C3-Th3—processus mastoideus, the processus spinosus Th3-Th5—processus transversus C1-C2, the processus transversus C3-Th6, the processus spinosus C3-Th1—linea nuchalis superior, the processus transversus Th1-Th6—processus spinosus C2-C7, the processus transversus C3-Th3—processus mastoideus, the processus transversus Th1-Th6—processus transversus C2-C6, the obliquus capitis inferior, the obliquus capitis superior, the processus spinosus C2-processus transversus C1, the suprasternal notch and clavicula (medial part)—processus mastoideus and linea nuchalis superior, the processus transversus C1-C4—scapula (angulus superior), the processus transversus C2-C7—first rib, the processus transversus C3-C6—first rib, the longus capitis, the processus transversus C3-C6—occipital bone (basilar part), the longus colli, the semispinalis cervicis, the spinalis capitis, the rectus capitis posterior major, the rectus capitis posterior minor, the rectus capitis anterior, the multifudus, and/or the processus transversus C2-C5—atlas (anterior tubercle).

44. The modified BoNT/A for use, method, or use according to any one of the preceding claims, wherein a single unit dose of modified BoNT/A is administered to one or more affected neck muscle(s) selected from a first group comprising: M. splenius cervicis, M. obliquus capitis inferior, M. semispinalis cervicis, M. scalenus (e.g. M. scalenus anterior, medius, and/or posterior), M. longissimus capitis, M. longus colli, and/or M. longus capitis; and/or a single or multiple unit doses of modified BoNT/A are administered to one or more affected neck muscle(s) selected from a second group comprising: M. splenius capitis, M. longissimus cervicis, M. trapezius (e.g. M. trapezius pars descendens), M. sternocleidomastoideomispinalis capitis, and/or M. levator scapulae.

45. The modified BoNT/A for use, method, or use according to any one of the preceding claims, wherein a single unit dose of modified BoNT/A is administered to one or more affected neck muscle(s) selected from a first group comprising: M. splenius cervicis, M. obliquus capitis inferior, M. semispinalis cervicis, M. scalenus (e.g. M. scalenus anterior, medius, and/or posterior), M. longissimus capitis, M. longus colli, and/or M. longus capitis; and/or multiple unit doses (preferably two unit doses) of modified BoNT/A are administered to one or more affected neck muscle(s) selected from a second group comprising: M. splenius capitis, M. longissimus cervicis, M. trapezius (e.g. M. trapezius pars descendens), M. sternocleidomastoideomispinalis capitis, and/or M. levator scapulae.

46. The modified BoNT/A for use, method, or use according to claim 44 or 45, wherein the unit dose is:
(a) 20,000 pg to 30,000 pg, such as 24,000 to 26,000 pg (preferably 25,000 pg) of modified BoNT/A; or
(b) 30,000 pg to up to 40,000 pg, such as 35,000 pg to 37,000 pg, (preferably 36,000 pg) of modified BoNT/A.

47. The modified BoNT/A for use, method, or use according to any one of claims 44-46, wherein the total number of unit doses administered during the treatment is up to 10 unit doses or up to 7 unit doses.

48. The modified BoNT/A for use, method, or use according to any one of the preceding claims, wherein the modified BoNT/A is administered by way of a unit dose per injection site at an affected neck muscle, or wherein the modified BoNT/A is administered by way of less than a unit dose per injection site.

49. The modified BoNT/A for use, method, or use according to any one of the preceding claims, wherein a single unit dose is administered at a plurality of injection sites at an affected neck muscle and/or two or more unit doses are administered at a plurality of injection sites at an affected neck muscle.

50. The modified BoNT/A for use, method, or use according to any one of the preceding claims, wherein a single unit dose is administered at an affected neck muscle (e.g. a single unit dose is administered at each affected neck muscle).

51. The modified BoNT/A for use, method, or use according to any one of the preceding claims, wherein cervical dystonia is treated for a longer duration than that treated by an unmodified BoNT/A (e.g. SEQ ID NO: 2 [such as a di-chain form of SEQ ID NO: 2]).

52. A unit dosage form of modified BoNT/A (e.g. for treating cervical dystonia), the unit dosage form comprising:
(a) greater than 707 Units of modified BoNT/A, wherein 1 Unit is an amount of the modified BoNT/A that corresponds to the calculated median lethal dose (LD₅₀) in mice; or
(b) greater than 17,000 pg of modified BoNT/A; and
(c) optionally a pharmaceutically acceptable carrier, excipient, adjuvant, and/or salt, wherein the modified BoNT/A comprises a BoNT/A light-chain and translocation domain, and a BoNT/B receptor binding domain (H_{C} domain).

53. A kit comprising:
(a) the unit dosage form according to claim 52; and
(b) instructions for use of the same in treating cervical dystonia; and
(c) optionally a diluent.


SEQUENCE LISTING (13 sequences)

SEQ ID NO: 1 - DNA, 3888 bp, Clostridium botulinum
  source 1..3888: mol_type = genomic DNA
atgccattcgtcaacaagcaattcaactacaaagacccagtcaacggcgtcgacatcgcatacatcaagattccgaacgccggtcaaatgcagccggttaaggcttttaagatccacaacaagatttgggttatcccggagcgtgacaccttcacgaacccggaagaaggcgatctgaacccgccaccggaagcgaagcaagtccctgtcagctactacgattcgacgtacctgagcacggataacgaaaaagataactacctgaaaggtgtgaccaagctgttcgaacgtatctacagcacggatctgggtcgcatgctgctgactagcattgttcgcggtatcccgttctggggtggtagcacgattgacaccgaactgaaggttatcgacactaactgcattaacgttattcaaccggatggtagctatcgtagcgaagagctgaatctggtcatcattggcccgagcgcagacattatccaattcgagtgcaagagctttggtcacgaggttctgaatctgacccgcaatggctatggtagcacccagtacattcgtttttcgccggattttaccttcggctttgaagagagcctggaggttgataccaatccgttgctgggtgcgggcaaattcgctaccgatccggctgtcacgctggcccatgaactgatccacgcaggccaccgcctgtacggcattgccatcaacccaaaccgtgtgttcaaggttaatacgaatgcatactacgagatgagcggcctggaagtcagcttcgaagaactgcgcaccttcggtggccatgacgctaaattcattgacagcttgcaagagaatgagttccgtctgtactactataacaaattcaaagacattgcaagcacgttgaacaaggccaaaagcatcgttggtactaccgcgtcgttgcagtatatgaagaatgtgtttaaagagaagtacctgctgtccgaggatacctccggcaagtttagcgttgataagctgaagtttgacaaactgtacaagatgctgaccgagatttacaccgaggacaactttgtgaaattcttcaaagtgttgaatcgtaaaacctatctgaattttgacaaagcggttttcaagattaacatcgtgccgaaggtgaactacaccatctatgacggttttaacctgcgtaacaccaacctggcggcgaactttaacggtcagaatacggaaatcaacaacatgaatttcacgaagttgaagaacttcacgggtctgttcgagttctataagctgctgtgcgtgcgcggtatcatcaccagcaaaaccaaaagcctggacaaaggctacaacaaggcgctgaatgacctgtgcattaaggtaaacaattgggatctgttcttttcgccatccgaagataattttaccaacgacctgaacaagggtgaagaaatcaccagcgatacgaatattgaagcagcggaagagaatatcagcctggatctgatccagcagtactatctgacctttaacttcgacaatgaaccggagaacattagcattgagaatctgagcagcgacattatcggtcagctggaactgatgccgaatatcgaacgtttcccgaacggcaaaaagtacgagctggacaagtacactatgttccattacctgcgtgcacaggagtttgaacacggtaaaagccgtatcgcgctgaccaacagcgttaacgaggccctgctgaacccgagccgtgtctataccttcttcagcagcgactatgttaagaaagtgaacaaagccactgaggccgcgatgttcctgggctgggtggaacagctggtatatgacttcacggacgagacgagcgaagtgagcactaccgacaaaattgctgatattaccatcattatcccgtatattggtccggcactgaacattggcaacatgctgtacaaagacgattttgtgggtgccctgatcttctccggtgccgtgattctgctggagttcattccggagattgcgatcccggtgttgggtaccttcgcgctggtgtcctacatcgcgaataaggttctgacggttcagaccatcgataacgcgctgtcgaaacgtaatgaaaaatgggacgaggtttacaaatacattgttacgaattggctggcgaaagtcaatacccagatcgacctgatccgtaagaaaatgaaagaggcgctggagaatcaggcggaggccaccaaagcaattatcaactaccaatacaaccagtacacggaagaagagaagaataacattaacttcaatatcgatgatttgagcagcaagctgaatgaatctatcaacaaagcgatgatcaatatcaacaagtttttgaatcagtgtagcgtttcgtacctgatgaatagcatgattccgtatggcgtcaaacgtctggaggacttcgacgccagcctgaaagatgcgttgctgaaatacatttacgacaatcgtggtacgctgattggccaagttgaccgcttgaaagacaaagttaacaataccctgagcaccgacatcccatttcaactgagcaagtatgttgataatcaacgtctgttgagcactttcaccgagtatatcaaaaacatcatcaatactagcattctgaacctgcgttacgagagcaatcatctgattgatctgagccgttatgcaagcaagatcaacatcggtagcaaggtcaattttgacccgatcgataagaaccagatccagctgtttaatctggaatcgagcaaaattgaggttatcctgaaaaacgccattgtctacaactccatgtacgagaatttctccaccagcttctggattcgcatcccgaaatacttcaacagcattagcctgaacaacgagtatactatcatcaactgtatggagaacaacagcggttggaaggtgtctctgaactatggtgagatcatttggaccttgcaggacacccaagagatcaagcagcgcgtcgtgttcaagtactctcaaatgatcaacatttccgattacattaatcgttggatcttcgtgaccattacgaataaccgtctgaataacagcaagatttacatcaatggtcgcttgatcgatcagaaaccgattagcaacctgggtaatatccacgcaagcaacaacattatgttcaaattggacggttgccgcgatacccatcgttatatctggatcaagtatttcaacctgtttgataaagaactgaatgagaaggagatcaaagatttgtatgacaaccaatctaacagcggcattttgaaggacttctggggcgattatctgcaatacgataagccgtactatatgctgaacctgtatgatccgaacaaatatgtggatgtcaataatgtgggtattcgtggttacatgtatttgaagggtccgcgtggcagcgttatgacgaccaacatttacctgaactctagcctgtaccgtggtacgaaattcatcattaagaaatatgccagcggcaacaaagataacattgtgcgtaataacgatcgtgtctacatcaacgtggtcgtgaagaataaagagtaccgtctggcgaccaacgcttcgcaggcgggtgttgagaaaattctgagcgcgttggagatccctgatgtcggtaatctgagccaagtcgtggttatgaagagcaagaacgaccagggtatcactaacaagtgcaagatgaacctgcaagacaacaatggtaacgacatcggctttattggtttccaccagttcaacaatattgctaaactggtagcgagcaattggtacaatcgtcagattgagcgcagcagccgtactttgggctgtagctgggagtttatcccggtcgatgatggttggggcgaacgtccgctg

SEQ ID NO: 2 - protein, 1296 aa, Clostridium botulinum
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNIINTSILNLRYESNHLIDLSRYASKINIGSKVNFDPIDKNQIQLFNLESSKIEVILKNAIVYNSMYENFSTSFWIRIPKYFNSISLNNEYTIINCMENNSGWKVSLNYGEIIWTLQDTQEIKQRVVFKYSQMINISDYINRWIFVTITNNRLNNSKIYINGRLIDQKPISNLGNIHASNNIMFKLDGCRDTHRYIWIKYFNLFDKELNEKEIKDLYDNQSNSGILKDFWGDYLQYDKPYYMLNLYDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDQGITNKCKMNLQDNNGNDIGFIGFHQFNNIAKLVASNWYNRQIERSSRTLGCSWEFIPVDDGWGERPL

SEQ ID NO: 3 - protein, 1315 aa, synthetic construct
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKSEILNNIILNLRYKDNNLIDLSGYGAKVEVYDGVELNDKNQFKLTSSANSKIRVTQNQNIIFNSVFLDFSVSFWIRIPKYKNDGIQNYIHNEYTIINCMKNNSGWKISIRGNRIIWTLIDINGKTKSVFFEYNIREDISEYINRWFFVTITNNLNNAKIYINGKLESNTDIKDIREVIANGEIIFKLDGDIDRTQFIWMKYFSIFNTELSQSNIEERYKIQSYSEYLKDFWGNPLMYNKEYYMFNAGNKNSYIKLKKDSPVGEILTRSKYNQNSKYINYRDLYIGEKFIIRRKSNSQSINDDIVRKEDYIYLDFFNLNQEWRVYTYKYFKKEEMKLFLAPIYDSDEFYNTIQIKEYDEQPTYSCQLLFKKDEESTDEIGLIGIHRFYESGIVFEEYKDYFCISKWYLKEVKRKPYNLKLGCNWQFIPKDEGWTEHHHHHHHHHH

SEQ ID NO: 4 - protein, 1327 aa, synthetic construct
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNIIELGGGGSELSEILNNIILNLRYKDNNLIDLSGYGAKVEVYDGVELNDKNQFKLTSSANSKIRVTQNQNIIFNSVFLDFSVSFWIRIPKYKNDGIQNYIHNEYTIINCMKNNSGWKISIRGNRIIWTLIDINGKTKSVFFEYNIREDISEYINRWFFVTITNNLNNAKIYINGKLESNTDIKDIREVIANGEIIFKLDGDIDRTQFIWMKYFSIFNTELSQSNIEERYKIQSYSEYLKDFWGNPLMYNKEYYMFNAGNKNSYIKLKKDSPVGEILTRSKYNQNSKYINYRDLYIGEKFIIRRKSNSQSINDDIVRKEDYIYLDFFNLNQEWRVYTYKYFKKEEMKLFLAPIYDSDEFYNTIQIKEYDEQPTYSCQLLFKKDEESTDEIGLIGIHRFYESGIVFEEYKDYFCISKWYLKEVKRKPYNLKLGCNWQFIPKDEGWTEHHHHHHHHHH

SEQ ID NO: 5 - protein, 1314 aa, synthetic construct
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNILNNIILNLRYKDNNLIDLSGYGAKVEVYDGVELNDKNQFKLTSSANSKIRVTQNQNIIFNSVFLDFSVSFWIRIPKYKNDGIQNYIHNEYTIINCMKNNSGWKISIRGNRIIWTLIDINGKTKSVFFEYNIREDISEYINRWFFVTITNNLNNAKIYINGKLESNTDIKDIREVIANGEIIFKLDGDIDRTQFIWMKYFSIFNTELSQSNIEERYKIQSYSEYLKDFWGNPLMYNKEYYMFNAGNKNSYIKLKKDSPVGEILTRSKYNQNSKYINYRDLYIGEKFIIRRKSNSQSINDDIVRKEDYIYLDFFNLNQEWRVYTYKYFKKEEMKLFLAPIYDSDEFYNTIQIKEYDEQPTYSCQLLFKKDEESTDEIGLIGIHRFYESGIVFEEYKDYFCISKWYLKEVKRKPYNLKLGCNWQFIPKDEGWTEHHHHHHHHHH

SEQ ID NO: 6 - protein, 1304 aa, synthetic construct
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNILNNIILNLRYKDNNLIDLSGYGAKVEVYDGVELNDKNQFKLTSSANSKIRVTQNQNIIFNSVFLDFSVSFWIRIPKYKNDGIQNYIHNEYTIINCMKNNSGWKISIRGNRIIWTLIDINGKTKSVFFEYNIREDISEYINRWFFVTITNNLNNAKIYINGKLESNTDIKDIREVIANGEIIFKLDGDIDRTQFIWMKYFSIFNTELSQSNIEERYKIQSYSEYLKDFWGNPLMYNKEYYMFNAGNKNSYIKLKKDSPVGEILTRSKYNQNSKYINYRDLYIGEKFIIRRKSNSQSINDDIVRKEDYIYLDFFNLNQEWRVYTYKYFKKEEMKLFLAPIYDSDEFYNTIQIKEYDEQPTYSCQLLFKKDEESTDEIGLIGIHRFYESGIVFEEYKDYFCISKWYLKEVKRKPYNLKLGCNWQFIPKDEGWTE

SEQ ID NO: 7 - protein, 1304 aa, synthetic construct
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNILNNIILNLRYKDNNLIDLSGYGAKVEVYDGVELNDKNQFKLTSSANSKIRVTQNQNIIFNSVFLDFSVSFWIRIPKYKNDGIQNYIHNEYTIINCMKNNSGWKISIRGNRIIWTLIDINGKTKSVFFEYNIREDISEYINRWFFVTITNNLNNAKIYINGKLESNTDIKDIREVIANGEIIFKLDGDIDRTQFIWMKYFSIFNTELSQSNIEERYKIQSYSEYLKDFWGNPLMYNKEYYMFNAGNKNSYIKLKKDSPVGEILTRSKYNQNSKYINYRDLYIGEKFIIRRKSNSQSINDDIVRKEDYIYLDFFNLNQEWRVYTYKYFKKEEEKLFLAPISDSDEFYNTIQIKEYDEQPTYSCQLLFKKDEESTDEIGLIGIHRFYESGIVFEEYKDYFCISKWYLKEVKRKPYNLKLGCNWQFIPKDEGWTE

SEQ ID NO: 8 - protein, 1291 aa, Clostridium botulinum
MPVTINNFNYNDPIDNNNIIMMEPPFARGTGRYYKAFKITDRIWIIPERYTFGYKPEDFNKSSGIFNRDVCEYYDPDYLNTNDKKNIFLQTMIKLFNRIKSKPLGEKLLEMIINGIPYLGDRRVPLEEFNTNIASVTVNKLISNPGEVERKKGIFANLIIFGPGPVLNENETIDIGIQNHFASREGFGGIMQMKFCPEYVSVFNNVQENKGASIFNRRGYFSDPALILMHELIHVLHGLYGIKVDDLPIVPNEKKFFMQSTDAIQAEELYTFGGQDPSIITPSTDKSIYDKVLQNFRGIVDRLNKVLVCISDPNININIYKNKFKDKYKFVEDSEGKYSIDVESFDKLYKSLMFGFTETNIAENYKIKTRASYFSDSLPPVKIKNLLDNEIYTIEEGFNISDKDMEKEYRGQNKAINKQAYEEISKEHLAVYKIQMCKSVKAPGICIDVDNEDLFFIADKNSFSDDLSKNERIEYNTQSNYIENDFPINELILDTDLISKIELPSENTESLTDFNVDVPVYEKQPAIKKIFTDENTIFQYLYSQTFPLDIRDISLTSSFDDALLFSNKVYSFFSMDYIKTANKVVEAGLFAGWVKQIVNDFVIEANKSNTMDKIADISLIVPYIGLALNVGNETAKGNFENAFEIAGASILLEFIPELLIPVVGAFLLESYIDNKNKIIKTIDNALTKRNEKWSDMYGLIVAQWLSTVNTQFYTIKEGMYKALNYQAQALEEIIKYRYNIYSEKEKSNINIDFNDINSKLNEGINQAIDNINNFINGCSVSYLMKKMIPLAVEKLLDFDNTLKKNLLNYIDENKLYLIGSAEYEKSKVNKYLKTIMPFDLSIYTNDTILIEMFNKYNSEILNNIILNLRYKDNNLIDLSGYGAKVEVYDGVELNDKNQFKLTSSANSKIRVTQNQNIIFNSVFLDFSVSFWIRIPKYKNDGIQNYIHNEYTIINCMKNNSGWKISIRGNRIIWTLIDINGKTKSVFFEYNIREDISEYINRWFFVTITNNLNNAKIYINGKLESNTDIKDIREVIANGEIIFKLDGDIDRTQFIWMKYFSIFNTELSQSNIEERYKIQSYSEYLKDFWGNPLMYNKEYYMFNAGNKNSYIKLKKDSPVGEILTRSKYNQNSKYINYRDLYIGEKFIIRRKSNSQSINDDIVRKEDYIYLDFFNLNQEWRVYTYKYFKKEEEKLFLAPISDSDEFYNTIQIKEYDEQPTYSCQLLFKKDEESTDEIGLIGIHRFYESGIVFEEYKDYFCISKWYLKEVKRKPYNLKLGCNWQFIPKDEGWTE

SEQ ID NO: 9 - protein, 10 aa, synthetic construct
TKSLDKGYNK

SEQ ID NO: 10 - protein, 8 aa, synthetic construct
SLDKGYNK

SEQ ID NO: 11 - protein, 437 aa, synthetic construct
PFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSK

SEQ ID NO: 12 - protein, 439 aa, synthetic construct
PFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTK

SEQ ID NO: 13 - protein, 856 aa, synthetic construct
ALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNILNNIILNLRYKDNNLIDLSGYGAKVEVYDGVELNDKNQFKLTSSANSKIRVTQNQNIIFNSVFLDFSVSFWIRIPKYKNDGIQNYIHNEYTIINCMKNNSGWKISIRGNRIIWTLIDINGKTKSVFFEYNIREDISEYINRWFFVTITNNLNNAKIYINGKLESNTDIKDIREVIANGEIIFKLDGDIDRTQFIWMKYFSIFNTELSQSNIEERYKIQSYSEYLKDFWGNPLMYNKEYYMFNAGNKNSYIKLKKDSPVGEILTRSKYNQNSKYINYRDLYIGEKFIIRRKSNSQSINDDIVRKEDYIYLDFFNLNQEWRVYTYKYFKKEEMKLFLAPIYDSDEFYNTIQIKEYDEQPTYSCQLLFKKDEESTDEIGLIGIHRFYESGIVFEEYKDYFCISKWYLKEVKRKPYNLKLGCNWQFIPKDEGWTE